(19) United States
(12) Patent Application Publication
(10) Pub. No.: US 2004/0053989 A1
(43) Pub. Date: Mar. 18, 2004

(54) Dithiolthione compounds for the treatment of neurological disorders and for memory enhancement

(75) Inventors: Patrick T. Prendergast, Straffan (IE); Paul Armstrong, Belfast (GB)

Correspondence Address: BURR & BROWN, PO BOX 7068, SYRACUSE, NY, 13261-7068, US

(73) Assignee: Patrick Prendergast, Straffan (IE)

(21) Appl. No.: 10/612,476

(22) Filed: Jul. 2, 2003

Related U.S. Application Data
(63) Continuation of application No. 09/627,641, filed on Jul. 28, 2000, now abandoned.
(60) Provisional application No. 60/145,964, filed on Jul. 29, 1999.
(60) Provisional application No. 60/198,338, filed on Apr. 18, 2000.

(30) Foreign Application Priority Data
Apr. 13, 2000 (IE) 2000/0302
Apr. 13, 2000 (IE) 2000/0304

(51) Int. Cl. A61K 31/385; A61K 31/55; A61K 31/453; A61K 31/4025

(52) U.S. Cl. 514/440; 514/210.19; 514/217.03; 514/326; 514/422

(57) ABSTRACT

The invention provides methods to treat neurological disorders such as Alzheimer's disease, or to slow the progression of such diseases, or to treat and/or prevent other disorders as disclosed in the specification, by administering to patients, or delivering to the tissues of such patients, oltipraz or related compounds as disclosed in the specification.

### CROSS-REFERENCE TO RELATED APPLICATIONS

This application claims priority to the following copending applications: U.S. provisional application No.60/145,964 filed Jul. 29, 1999; U.S. provisional application No.60/198,338 filed Apr. 18, 2000; Irish patent application no.2000/0302 filed Apr. 13, 2000; and Irish patent application no. 2000/10303 filed Apr. 13, 2000; all of which are incorporated herein by reference.

### BACKGROUND OF THE INVENTION

1. Field of the Invention

This invention relates to methods of treating subjects who have, or who are at risk of having, a faulty memory, a degenerative disorder, a neurodegenerative disorder, a neurodegenerative-related disorder, or a parasite infection such as malaria, sleeping sickness or a trypanosome infection, using dithiolthione compounds or inhibitors of D-amino acid oxidase. This invention also relates to improved methods of making 1,2-dithiole-3-thiones, including oltipraz (CAS Number 6422-421-1). This invention also relates to a diagnostic assay for neurodegenerative disorders.

2. Description of the Related Art

Humanity is plagued by a wide variety of neurodegenerative disorders and neurodegenerative-related disorders, including Parkinson's disease, Huntington's disease, Amylotrophic Lateral Sclerosis, Cerebral amyloid angiopathy, Multiple Sclerosis, cognitive disorders, Progeria, Alzheimer's disease (“AD”), epileptic dementia, presenile dementia, post traumatic dementia, senile dementia, vascular dementia, HIV-1-associated dementia, post-stroke dementia, Down's syndrome, motor neuron disease, amyloidosis, amyloid associated with type 11 diabetes, Creutzfelt-Jakob disease, necrotic cell death, Gerstmann-Straussler syndrome, kuru and animal scrapie, amyloid associated with long-term hemodialysis, senile cardiac amyloid and Familial Amyloidotic Polyneuropathy, cerebropathy, neurospanchnic disorders, memory loss, aluminium intoxication, reperfusion injury, reducing the level of iron in the cells of living subjects, reducing free transition metal ion levels in mammals, patients having toxic amounts of metal in the body or in certain body compartments, and related degenerative disorders.

Many neurodegenerative disorders and neurodegenerative-related disorders are both difficult to treat and difficult to diagnose. For instance, criteria for the diagnosis of probable Alzheimer's Disease have been described and include: (1) the presence of a dementia syndrome with defects in two or more areas of cognition; (2) progressive worsening of memory and other cognitive function over time; (3) a relatively intact level of consciousness (4) age at disease onset at a time between 40 and 90 years of age; and (5) the specific absence of any other systemic or central nervous system process that could account for the progressive cognitive deterioration in the individual.

In addition, the probability of an accurate diagnosis in the living patient is augmented by laboratory examinations and by imaging studies (such as computed tomography and magnetic resonance imaging). Such laboratory examinations and/or imaging studies demonstrate the existence and effects of other causes of dementia (such as subdural hematoma, intracranial tumours, infection and brain infarction) and disclose results that are consistent with but are not themselves diagnostic of Alzheimer's disease. The best clinical diagnosis available to date is only a presumptive determination based on criteria that are evaluations of cognitive and neurological functions for that patient.

U.S. Pat. No. 6,027,896 discloses a method of diagnosing and prognosing Alzheimer's disease. This method is based on the fact that senile plaque and congophilic angiopathy are abnormal extracellular structures found in abundance in brain of patients with Alzheimer's disease. U.S. Pat. No. 5,972,634 discloses an ELISA assay for detecting Aβ peptide, using solid supports coated with heavy metal cations and antibodies to Aβ peptide. However, there remains a need for improved methods to diagnose and prognose neurodegenerative disorders, including Alzheimer's disease.

AU701953 (Masters) discloses a method of treating Alzheimer's disease. AU701953 teaches that iron is not relevant to the heparin-binding site, which is hypothesized to be altered in Alzheimer's disease.

WO9827970 (Fiander et al) discloses the use of Michael reaction acceptors, for use in protecting cells against the toxic effects of oxygen containing free radicals in mammals. WO9827970 does not teach the use of compounds that inhibit DAAO, chelate iron and/or copper or enhance phase II detoxification enzymes in prophylaxis and treatment of degenerative disorders including Alzheimer's disease.

U.S. Pat. No. 5,668,117 (Shapiro) is directed to the methods of treatment of neurological diseases (Alzheimer's disease, Parkinson's disease, ALS listed) using carbonyl trapping agents in combination with previously known medicaments. Oltipraz is listed as being useful to use in combination due to its facilitation of glutathione activity.

A major focus of AD-related research focuses on amyloid-β. Amyloid-β deposits are often found in regions of the brain that are susceptible to the neurodegenerative processes. Production of amyloid-β is increased in inherited forms of AD. Also, amyloid-β in tissue culture is toxic to neurons and clonal cell lines. The neurotoxic activity of amyloid-β is dependent upon its aggregation into fibrils with a high content of β-sheet secondary structure. Its toxicity is mediated by oxidative stress, which is attenuated by anti-oxidants. Recently, it has been found that the toxicity of amyloid-β is mediated by iron. The toxicity was attenuated in a dose-dependent fashion by deferoxamine and restored, again in a dose-dependent fashion, by subsequent exogenous addition of ferrous iron. Thus, an iron chelating agent could be suitable for use in AD and related neurodegenerative conditions, including early in the onset of such conditions.

AD appears to alter many aspects of brain homeostasis. The pathological presentation of AD, the leading cause of senile dementia, involves regionalized neuronal death and an accumulation of intraneuronal and extracellular lesions termed neurofibrillary tangles and senile plaques, respectively (reviewed in Smith, 1998). Several independent hypotheses have been proposed to link the pathological lesions and neuronal cytopathology with, among others, apolipoprotein E genotype (Corder et al. 1993; Roses, 1995), hyper-phosphorylation of cytoskeletal proteins (Trojanowski et al. 1993), and amyloid-β metabolism (Selkoe, 1997). However, not one of these theories alone is sufficient to explain the diversity of abnormalities found in AD that involves a multitude of cellular and biochemical changes. Furthermore, attempts to mimic AD by a perturbation of one of these elements using cell or animal models, including transgenic animals, do not result in the same spectrum of pathological alterations. Perhaps the most striking example of this is that while amyloid-β plaques are deposited in some transgenic rodent models overexpressing β-protein precursor, there is little (Staufenbiel et al., 1998) or no (Irizarry et al. 1997a,b) neuronal loss—a seminal feature of AD.

Oxidative damage and responses to such damage occur in AD. The overall result of unchecked oxygen radicals is damage. Such damage found in AD includes advanced glycation end products (Smith et al. 1994a; Ledesma et al. 1994; Vitek et al. 1994; Yan et al. 1994), nitration (Good et al. 1996; Smith et al. 1997a), lipid peroxidation adduction products (Montine et al. 1996a; Sayre et al. 1997a) as well as carbonyl-modified neurofilament protein and free carbonyls (Smith et al. 1991; Smith et al. 1995, 1996). Importantly, this damage involves all neurons in populations vulnerable to death in AD, not just those containing neurofibrillary tangles. In fact, the exact spatiotemporal distribution of specific types of damage elegantly reflects the biology and chemistry of each modification.

The cytopathological significance of oxidative damage is seen by the upregulation of heme oxygenase-1, an enzyme that not only converts heme to an antioxidant but also yields free iron (Smith et al. 1994b; Schipper et al. 1995; Premkumar et al. 1995), in vulnerable neurons. Quantitative immunocytochemical studies of cases of AD show that there is a complete overlap between neurons upregulating heme oxygenase-1 and Alz50, an early marker of oxidative abnormalities, indicating that cytoskeletal abnormalities are associated with heme oxygenase induction or vice versa. Significantly, the Alz50 epitope predates the formation of Congo red positive neurofibrillary tangles.

A number of mechanisms have been suggested to explain the neurotoxicity of amyloid-β (Yankner et al. 1990; reviewed in Iversen et al. 1995; Sayre et al. 1997b) including membrane depolarization (Carette et al. 1993), increased sensitivity to excitotoxins (Koh et al. 1990), and alterations in calcium homeostasis (Mattson et al. 1992), however, the influences of amyloid-β and other genetic factors on AD may be through their effect on oxidative stress. Neuronal damage in vitro by amyloid-β is mediated by free radicals and, as such, can be attenuated by using antioxidants such as vitamin E (Behl et al. 1992, 1994) or catalase (Lockhart et al. 1994; Zhang et al. 1996). Further, mutations in β-protein precursor are associated with increased DNA fragmentation, possibly involving oxidative mechanisms (Perry et al., 1998a,b).

Presenilins 1 and 2 (Sherrington et al. 1995; Selkoe, 1997) are genetic factors where the biological mechanism, although not established, may also involve oxidative damage. Increased presenilin 2 expression increases DNA fragmentation and apoptotic changes (Wolozin et al. 1996), both important consequences of oxidative damage. Apolipoprotein E, in brain and cerebrospinal fluid, is found adducted with the highly reactive lipid peroxidation product, hydroxynonenal (Montine et al. 1996b). Furthermore, apolipoprotein E is a strong chelator of copper and iron, important redox-active transition metals (Miyata and Smith, 1996).

A question relevant to AD is what the initial source of increased reactive oxygen production is. Reactive oxygen is a ubiquitous byproduct of both oxidative phosphorylation and the myriad of oxidases necessary to support aerobic metabolism. In AD, in addition to this background level of reactive oxygen, there are a number of additional contributory sources that are thought to play an important role in the disease process: (1) Iron, in a redox-active state, is increased in neurofibrillary tangles as well as in amyloid-β deposits (Good et al. 1992; Smith et al. 1997b). Iron catalyzes the formation of .OH from H₂O₂ as well as the formation of advanced glycation end products. Furthermore, aluminum, which also accumulates in neurofibrillary tangle-containing neurons (Good et al. 1992), stimulates iron-induced lipid peroxidation (Oteiza, 1994); (2) Activated microglia, such as those that surround most senile plaques (Cras et al. 1990), are a source of NO and O₂⁻. (Colton and Gilbert, 1987) which can react to form peroxynitrite, leaving nitrotyrosine as an identifiable marker (Good et al. 1996; Smith et al. 1997a); (3) Amyloidβ itself has been directly implicated in reactive oxygen formation through peptidyl radicals (Butterfield et al. 1994; Hensley et al. 1994; Sayre et al. 1997b); (4) Advanced glycation end products in the presence of transition metals (see above) can undergo redox cycling with consequent reactive oxygen species production (Baynes, 1991; Yan et al. 1994, 1995). Additionally, advanced glycation end products, as well as amyloid-β, activate specific receptors, such as the receptor for advanced glycation end products (RAGE) and the class A scavenger-receptor, to increase reactive oxygen production (Yan et al. 1996; E I Khoury et al. 1996); (5) Abnormalities in the mitochondrial genome (Corral-Debrinski et al. 1994; Davis et al. 1997) or deficiencies in key metabolic enzymes (Sorbi et al. 1983; Sheu et al. 1985; Sims et al. 1987; Blass et al. 1990; Parker et al. 1990) suggest that metabolic abnormalities affecting mitochondria may be the major and possibly initiating source of reactive oxygen in AD.

As discussed in detail below, given that oxidative damage occurs prior to the appearance of other abnormalities, it is unlikely that Aβ, advanced glycation end products or microglia are primary contributors. However, redox-active iron, especially in conjunction with mitochondrial abnormalities, represent an early and, equally importantly, cytoplasmic base for the generation of oxidizing species.

Oxidative damage precedes the lesions in AD and is restricted to cell bodies of vulnerable neurons. In order to address where reactive oxygen species are produced, efforts were centered on finding a marker resulting from primary attack, rather than more complex secondary reactions, and that involves damage to a cell constituent with short half-life. Proteins fail in the latter aspect because modifications associated with crosslinking slow their turnover. Therefore, crosslink modifications of proteins, while useful to assess history, may reveal less of the current state. However, 8-hydroxyguanosine (8 OHG), a nucleic acid modification predominantly derived from .OH attack of guanidine, is greatly increased in cytoplasmic RNA in vulnerable neuronal populations (Nunomura et al., 1999a). 8 OHG is likely to form at the site of .OH production, a process dependent on redox-active metal catalyzed reduction of H₂O₂ with cellular reductants such as ascorbate or O₂⁻ (FIG. 3).

The pharmacotherapy of Alzheimer's disease has led to a large number of clinical trials involving a wide variety of drugs. Most studies to date have involved attempts to enhance the effects of the damaged cholinergic system. Other strategies include blocking over-stimulation of excitatory amino acid (especially glutamate) receptors, blocking the influx of Ca²⁺, and removing free radicals and other oxidants. Another way to enhance cholinergic function is to supply acetylcholine precursors. Choline and phosphatidylcholine (lecithin) have been used in attempts to augment acetylcholine synthesis, in an analogous way to the use of a dopaminergic precursor (L-dopa) in Parkinson's disease. While, at least in animal studies, cholinergic precursors, such as choline and lecithin, can increase levels of acetylcholine and, in certain circumstances, even enhance cholinergic transmission, numerous human trials, however, have generally yielded negative or inconclusive results. Better methods for treating neurodegenerative disorders such as Alzheimer's disease are needed.

1,2-Dithiole-3-thiones have not been previously used for the treatment of neurodegenerative disorders, including Alzheimer's disease. Oltipraz is a 1,2-dithiole-3-thione having the following structure:

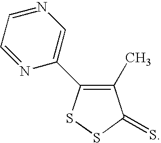

A method of making oltipraz is disclosed in U.S. Pat. No. 4,110,450. However, overall yields in that process are not particularly high and the starting material for that process, pyrazine methyl ester, is relatively expensive as compared to the corresponding pyrazine carboxylic acid. Therefore, there is a need for an improved method for making oltipraz and related 1,2-dithiole-3-thiones.

### SUMMARY OF THE INVENTION

In a principal embodiment, the invention provides a method to treat, prevent or slow the progression of a degenerative disorder, a neurodegenerative disorder, impaired memory, a neurodegenerative-related disorder, malaria, or a trypanosome infection, or to ameliorate a symptom thereof, or to treat aluminum intoxication, reperfusion injury, or to reduce the level of iron or to reduce free transition metal ion levels in the body or in certain body compartments, in a subject in need thereof, the method comprising administering to the subject or delivering to the subject's tissues a therapeutically effective amount of a compound having the formula

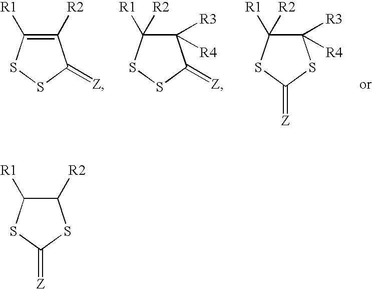

and oxides, derivatives and metabolites thereof, wherein

Z is S, O, NR, R₂ or CR₂;

R is —H, —OH, C₁-C₅ alkyl, C₁-C₅ alkoxy or C₁-C₅ alkoxycarbonyl;

R₂, together with the atoms to which it is bonded, comprise a spiro ring;

R1, R2, R3 and R4 independently are —H, -alkyl, -aryl, -alkylaryl, a heterocycle, a halogen, -alkoxycarbonyl (C₁-C₅) or -carboxyl,

wherein either alkyl is a C₁-C₁₀ linear or branched chain, saturated or unsaturated moiety, which is optionally substituted by 1, 2 or more independently selected ether (—O—), halogen, alkyl (C₁-C₅), —OH, alkoxy (C₁-C₅), alkoxycarbonyl, (C₁-C₅), carboxyl, amido, alkyl amido (C₁-C₅), amino, mono- or dialkylamino (C₁-C₅), alkyl carbamoyl (C₁-C₅), thiol, alkylthio (C₁-C₅), or benzenoid aryl, and

wherein the -aryl and -alkylaryl substituent for R1, R2, R3 and R4 comprises a benzenoid group (C₆-C₁₄), wherein the benzenoid group is optionally substituted with 1, 2 or more independently selected —SO₃H, halogen, alkyl (C₁-C₅), —OH, alkoxy (C₁-C₅), alkoxycarbonyl, (C₁-C₅), carboxyl, amido, alkyl amido (C₁-C₅), amino, mono- or dialkylamino (C₁-C₅), alkyl carbamoyl (C₁-C₅), thiol, alkylthio (C₁-C₅), and

wherein the heterocycle is defined as any 4, 5 or 6 membered, optionally substituted heterocyclic ring, saturated or unsaturated, containing 1-3 ring atoms selected from N, O and S, the remaining ring atoms being carbon; and wherein said substituents on said aryl or said heterocyclic are selected from the group consisting of halogen, alkyl (C₁-C₅), hydroxyl, alkoxy (C₁-C₅), alkoxycarbonyl (C₁-C₅), carboxyl, amido, alkyl amido (C₁-C₅), amino, mono and dialkyl amino (C₁-C₅), alkyl carbamoyl (C₁-C₅), thiol, alkylthio (C₁-C₅), benzenoid, aryl, cyano, nitro, haloalkyl (C₁-C₅), alklsulfonyl (C₁-C₅), or sulfonate, or

one of R1 and R2 and one of R3 and R4 together with the carbon atoms to which they are attached comprise a fused bicyclic or tricyclic compound, which is saturated or unsaturated, heterocyclic or carbocyclic and wherein the rings are all optionally substituted 5-, 6-, 7- or 8-membered rings, with substituents optionally selected from alkyl, alkoxy, —SO₃H, —OH and halogen, or

R1 and R2 together or R3 and R4 together independently are oxime (═NOH).

In another embodiment, the invention provides a method to determine if a mammal has a neurodegenerative or related disorder or the propensity to develop such a disorder, comprising: (a) obtaining a circulatory fluid sample from the mammal; (b) splitting the circulatory fluid sample into two, three or more suitable aliquots; (c) determining the hydrogen peroxide level in a first aliquot; (d) contacting a second aliquot with a sufficient amount of a one, two or more D-amino acids; (e) incubating the second aliquot for sufficient time and under conditions suitable to allow detectable metabolism of the one, two or more D-amino acids to determine the level of hydrogen peroxide in the second aliquot; (f) determining the hydrogen peroxide level of second first aliquot; and (g) comparing the hydrogen peroxide level obtained from step (c) and step (f) and the, whereby a high hydrogen peroxide level indicates the presence of a neurodegenerative or related disorder or the propensity to develop such a disorder.

Related embodiments provide a method to make oltipraz comprising (1) contacting pyrazine-2-carboxylic acid with methanol in the presence of an acid to form methyl-pyrazine-2-carboxylate; (2) condensing the methyl-pyrazine-2-carboxylate with methyl propionate in the presence of a base to form methyl-2-methyl-3-(pyrazin-2-yl)-3-oxopropionate; and (3) treating said methyl-2-methyl-3-(pyrazin-2-yl)-3-oxopropionate with phosphorus pentasulfide to form oltipraz.

Additional embodiments are described in the following discussion.

### DETAILED DESCRIPTION OF THE PREFERRED EMBODIMENTS

Definitions.

The following terms have the meanings given below, unless expressly stated otherwise or implied otherwise by context.

Alkyl means a C1-C10 moiety that is linear or branched, saturated or unsaturated which can be optionally substituted by 1, 2, 3 or more independently selected halogen, alkyl (C1-C5), hydroxyl, alkoxy (C1-C5), alkoxycarbonyl, (C1-C5), carboxyl, amido, alkyl amido (C1-C5), amino, mono and dialkyl amino (C1-C5), alkyl carbamoyl (C1-C5), thiol, alkylthio (C1-C5) or benzenoid aryl. Alkyl, as used herein, includes aliphatic and cyclic organic residues having a carbon at a point of attachment. Accordingly, alkyl groups include unsubstituted hydrocarbon residues of the formula CₙH₂ₙ₊₁ and substituted and cyclic forms thereof. Such hydrocarbons are usually of the lower alkyl class, which have six carbons or less. It is understood that larger alkyl groups may be used. Alkyl includes substituted residues which are intended to include the hydrocarbon residues bearing one or more, same or different, functional groups as described below.

Aryl means an optionally singly or multiply substituted benzenoid group (C6-C14), e.g., phenyl, naphthyl. Aryl, as used herein, includes organic residues derived from aromatic hydrocarbon or aromatic heterocyclic ring systems. Accordingly aryl groups include the unsubstituted ring residues such as phenyl and naphthyl and substituted derivatives.

The alkyl and aryl group previously described may be substituted with functional groups. Such functional groups include essentially all chemical groups which can be introduced synthetically and result in stable compounds. Examples of these functional groups are hydroxyl, halogen (fluoro, chloro, bromo), amino (including alkylamino and dialkylamino), cyano, nitro, carboxy (including carbalkoxy), carbamoyl (including N and N,N alkyl), thiol, alkoxy, alkyl, aryl, and arylazo.

Heteroaryl or heterocycle means a 4, 5 or 6 membered saturated or unsaturated ring that comprises 1, 2 or 3 N, O or S atoms in each ring, the remaining ring atoms being carbon. Heterocyclic or heteroaryl residues may be those comprising one or more heteroatoms (e.g., nitrogen, oxygen, sulfur) in the ring system such as pyridyl, oxazolyl, quinoly), thiazolyl and substituted forms thereof. Heterocycles are optionally substituted.

Substituents that are bonded to aryl or heterocycles include 1, 2, 3 or more of halogen, alkyl (C1-C5), hydroxyl, alkoxy (C1-C5), alkoxycarbonyl, (C1-C5), carboxyl, amido, alkyl amido (C1-C5), amino, mono and dialkyl amino (C1-C5), alkyl carbamoyl (C1-C5), thiol, alkyl thio (C1-C5) or benzenoid aryl, cyano, nitro, haloalkyl, alklsulfonyl (C1-C5), sulfonate, or two of such substituents can be part of a fused ring, which can be either saturated, or unsaturated, heterocyclic or carbocyclic. When more than one substituent is present on a molecule, they can be the same or independently selected.

Halogen or halo means fluorine, chlorine, bromine or iodine. When more that one halogen is present, each can be the same or independently selected.

Invention Embodiments.

The treatment methods of the instant invention comprise identifying a subject having a faulty memory, a degenerative disorder, a neurodegenerative disorder, a neurodegenerative-related disorder, malaria, or a trypanosome infection, or a subject at risk of developing a faulty memory, a neurodegenerative disorder, a neurodegenerative-related disorder, malaria, a Leishmania infection, or a trypanosome infection. These subjects may be identified by methods well known to those skilled in the art or by the methods disclosed herein.

Although the instant invention is not bound by any theory, it is believed that with aging, the D-amino acid percentage in a subject increases so that a larger percentage of amino acids present in the cell will be D-amino acids. The proteins thus formed will then be made up of a percentage of D-amino acids and will not function. The body produces D-amino acid oxidase (DAAO) to metabolize them, producing ammonia and hydrogen peroxide, which are toxic to cells. In young cells, the phase II detoxification enzymes, e.g., glutathione reductase, are present in sufficient quantity to combat hydrogen peroxide production, but their levels are thought to reduce with aging. The compounds of the instant invention, particularly oltipraz, are believed to inhibit DAAO and thus enhance the effect of glutathione reductase enzymes. Other explanations are possible and are mentioned herein.

The compounds of the instant invention include all those described herein. Preferred compounds of the instant invention include oltipraz, 5-(4-methoxyphenyl)-3H-1,2-dithiole-3-thione (anetholetrithione), ADT, ADO, 1,2-dithiole-3-thione, 1,2-dithiolane, 1,3-dithiole-2-thione, and malotilate.

The instant invention also provides an improved method for making 1,2-dithiolanes such as oltipraz. The details of the improved method for the synthesis of oltipraz are described below. This synthesis proceeds in three basic steps: (a) esterifying pyrazine-2-carboxylic acid with methanol in the presence of an acid, preferably sulfuric acid, to form methyl-pyrazine-2-carboxylate; (b) condensing said methyl-pyrazine-2-carboxylate with methyl propionate in the presence of a base, preferably potassium hydride, more preferably sodium hydride, to form methyl-2-methyl-3-(pyrazin-2-yl)-3-oxoproprionate; and (c) treating said methyl-2-methyl-3-(pyrazin-2-yl)-3-oxopropionate with an inorganic sulfide, preferably phosphorus pentasulfide, to form oltipraz. Preferably, steps (b) and (c) are conducted in the presence of an aromatic hydrocarbon, most preferably toluene. Those skilled in the art may modify the procedure described herein for the synthesis of oltipraz by following the teachings disclosed herein in a manner known to those skilled in the art to produce other 1,2-dithiolanes, preferably other 1,2-dithiolanes as disclosed herein.

The instant invention also provides a method for determining that a subject, preferably a mammal, most preferably a human, has a neurodegenerative or neurodegenerative-related disorder, preferably to determine Alzheimer's disease. This method comprises the steps of: (a) obtaining a circulatory fluid, preferably blood or spinal fluid, comprised of serum from the subject; (b) removing at least a part of the serum from the circulatory fluid to obtain separated serum; (c) splitting the separated serum into at least a first separated serum sample and a second separated serum sample; (d) determining the level of hydrogen peroxide or the level of ammonia in the first separated serum sample; (e) treating the second separated serum sample with a D-amino acid to form a treated serum sample; (f) incubating the treated serum sample; (g) determining the level of hydrogen peroxide or ammonia in the treated serum sample; and (h) comparing the level of hydrogen peroxide or ammonia in the first separated serum sample to the level of hydrogen peroxide or ammonia in the treated serum sample.

Any of the compounds disclosed herein are suitable for use to treat the conditions or diseases disclosed herein, or to ameliorate one or more symptoms associated with those conditions or diseases or to slow the progression or accumulation of damage or symptoms associated therewith (e.g., memory loss, disorientation or any of the symptoms disclosed herein or in the cited references). For example, the compounds the compounds are useful to slow, e.g., neurological damage and/or cognitive deterioration associated with the neurological disorders disclosed herein (e.g., AD). The compounds can be used to treat or to slow progression of diseases, infectious agents or parasite agents such as malaria (Plasmodium parasites such as P. falciparum, P. vivax, P. berghi), Trypanosome parasites (e.g., T. cruzi, T. rhodesiense), Leishmania parasites (e.g., L. donovani), sleeping sickness, Chagas disease, Mycoplasma, hairy Leukoplakia, oral candidosis, mouth ulcerations-aphthous/herpetic/bacterial, fungal candida, human papilloma viruses, molluscum contagiosum, squamous oral carcinoma, Kaposi's sarcoma oral lesions, periodontitis, necrotizing gingivitis, orafacial herpes zoster, and rotaviruses. Treatment of the infections is optionally combined with other conventional or experimental treatments, e.g., antiviral or antifungal agents. These infectious agents may be sensitive or resistant to conventional treatments.

Accordingly, the present invention provides a method for treating these infections which comprises administering to an afflicted host a therapeutically effective amount of a compound (or a pharmaceutically acceptable salt thereof) of the present invention, as well as derivatives, metabolites, and precursors thereof, as defined herein. Preferably, the afflicted host is first identified as having the infection.

This invention provides methods to treat memory impairment or to enhance memory or to slow the rate of memory impairment or neuron.or tissue damage in patients suffering from AD, Parkinson's disease, Huntington's disease, Amylotrophic Lateral Sclerosis, Cerebral amyloid angiopathy, Multiple Sclerosis, cognitive disorders, Progeria, epileptic dementia, presenile dementia, post traumatic dementia, senile dementia, vascular dementia, HIV-1 -associated dementia, post-stroke dementia, Down's syndrome, motor neuron disease, amyloidosis, amyloid associated with type II diabetes, Creutzfelt-Jakob disease, necrotic cell death, Gerstmann-Straussler syndrome, kuru and animal scrapie, amyloid associated with long-term hemodialysis, senile cardiac amyloid and Familial Amyloidotic Polyneuropathy, cerebropathy, neurospanchnic disorders, memory loss, aluminum intoxication, reperfusion injury, the methods comprising administering to the patient an effective amount of a compound of the invention. The compounds of the invention can also be used in the same manner to reduce the level of iron in the cells of living subjects, reduce free transition metal ion levels in mammals having toxic amounts of metal in the body or in certain body compartments, and related degenerative disorders. In a further aspect, the present invention relates to compositions and formulations useful in any of the methods disclosed herein. Collectively, the conditions that can be treated are referred to as degenerative disorders, some of which are associated with neural tissue diseases or disorders, e.g., AD. These latter conditions are referred to as neurodegenerative or neurodegenerative-related disorders or the like.

The present invention also provides methods for reducing iron levels in mammals by administration of an effective amount of one or more of the compounds identified herein to a mammal in need thereof. The present invention relates to the treatment of hosts suffering from iron overload or non-Iron overload diseases and/or conditions, such as thalassemia, anemia, hereditary hemochromatosis, hemodialysis or stroke. In a further aspect, the present invention relates to compositions and formulations useful in the methods disclosed herein.

This invention provides a process for using an amount of compounds disclosed in the attached embodiments. It is an object of this invention to employ an effective dosage of oltipraz for arresting disease process and substantially enhancing the memory function.

It is an object of the present invention to employ one or more compounds as identified in the embodiments disclosed in the specification or in the claims for use in prophylactically treating a patient for any form of neuronal or cognitive deficiency, e.g., as disclosed herein or in the cited references.

Raised iron levels promote the oxidation of catecholamines via quinone intermediates and free radical toxicity. The endogenous opioid may cause suppression of proenkephalin. A transcription or influence its posttranscriptional regulation, and also affect dopamine and other amine storage in vesicles and postsynaptic effect.

In Parkinson's disease, excess iron, the low scavenging enzyme activities, inhibition of mitochondrial metabolism morphological damage to mitochondria and damage to enkephalinergic pathways result from mitochondrial DNA damage and proteolysis of non-functional cytochromes translated there from. The mitochondrial DNA environment, its damage and lack of a pyrimidine dimer repair system predisposes patients to the development of Parkinson's disease. Plaque and tangle formation observed in Parkinson's disease, AD, Amylotrophic Lateral Sclerosis, resulting there from are caused by the release of iron, copper and calcium, which activate metal endopeptidases.

Parkinson's disease may result from age related DNA damage in mitochondria caused by accumulation of free radicals, xenobiotics, dopamine, quinones, radiation, and age related decline in polyamine levels. Copper is particularly active in promoting xenobiotic induced DNA base damage. Paraquat and polyamines, putrescine and spermidine show reciprocal competitive inhibition of uptake.

A 5000 base pair deletion has been observed in some areas of the brain during aging and in Parkinson's disease (Ikebe S. et al). A single base pair mutation or deletion at any of several sites can cause complex 1 deficiency in mitochondrial myopathy patients (Holt L. J. et al). The significance of these deletions, though likely rare amongst all Parkinson's disease cases is that random DNA base damage can produce a similar pattern of disease.

Disturbance of cytochrome regulation would lead to the iron and opioid defects. Excessive transcription, excessive translation of a normal mitochondrial transcript or an abnormally sequenced or spliced one, or excessive intra-mitochondrial proteolysis would serve as a source of raised intra-mitochondrial iron, raised intracellular iron and an endogenous opioid, cytocrophin.

Free metals in vivo activate metal dependent endopeptidases such as the physiological precursor cleaving peptidases i.e. non-lysosomal proteases, and calpains. They can be implicated in the pathological changes of dementias, including beta amyloid and neurofibrillary tangle formation, and demyelination. The molecules involved in generating Lewy bodies, Hirano bodies, Pick bodies, and granulovacuolar degeneration are not known at the present time. Brain copper levels are highest in locus coeruleus, substantia nigra, putamen and globus pallidum respectively. Brain iron levels are highest in globus pallidum, putamen and substantia nigra respectively. Release of metals at particular subcellular sites is likely a common event in the pathogenesis of Alzheimer's, Parkinson's, Batten's, Pick's dementias and dialysis aluminum induced encephalopathy. Agents that influence subcellular compartmentation and distribution of copper, iron, nickel and aluminum offer therapeutic prospects in preventing these pathologies or in reducing more symptoms associated therewith. Enzyme inhibition of pre aspartate proteases may not be therapeutically practicable as these proteases serve physiological functions. Regulation of the peptide precursor cleaving enzyme activities by control of free metal levels is an interesting therapeutic avenue. Their significance in dementia pathogenesis is likely due to the absence of other enzyme classes, capable of cleaving at pre aspartate sites.

High levels of Cu or Zn have been previously demonstrated immunohistochemically in the large pyramidal cells of control and Alzheimer's disease patients' brains. The localization of the superoxide dismutase gene on chromosome twenty-one and the early occurrence of Alzheimer's Disease in Down's syndrome suggest that superoxide dismutase activity and hydrogen peroxide formation may contribute to Alzheimer's pathogenesis. Also the neurons containing high levels of NADPH diaphorase are relatively spared in neonatal hypoxia and hypoglycemia but are affected in Alzheimer's disease. The increase in platelet membrane fluidity, noted in a subgroup of Alzheimer's disease patients, possibly due to disregulation of platelet membrane biosynthesis is not associated with a higher erythrocyte level of superoxide dismutase.

Iron is deposited as haemosiderin granules in the cytoplasm, and mitochondria filled with ferritin granules have been observed in the neuronal and glial cells of the ventrolateral thalamus, caudate and lenticular nuclei and substantia nigra of Parkinson's brains (Earle K. M., Asenjo A. et. al., Riederer P. et. al.), and copper, though not detectable in excess in the brain, does overflow into the cerebrospinal fluid. The level of copper overflow correlates with the clinical severity of Parkinson's disease and the level of Alzheimer type damage present in the patients (Pall H. S. et al).

Though Parkinsonian syndromes can be induced by other metals such as chronic manganese poisoning which causes Parkinsonian like and psychotic symptoms in miners and hepatolenticular degeneration due to copper deposition in Wilson's Disease, excessive levels of metals other than iron have not been observed in idiopathic or post encephalitic Parkinsonism.

Unchecked oxygen radicals cause damage of various types. Importantly, this damage involves all neurons in populations vulnerable to death in Alzheimer's disease, not just those containing neurofibrillary tangles.

Reactive oxygen is a ubiquitous by product of both oxidative phosphorylation and the myriad of oxidases necessary to support aerobic metabolism. In AD, in addition to this background level of reactive oxygen, there are a number of additional contributory sources that are thought to play an important role in the disease process: (1) Iron, in a redox-active state, is increased in neurofibrillary tangles as well as in amyloid-β. deposits. Iron catalyzes the formation of .OH from H₂O₂ as well as the formation of advanced glycation end products. Furthermore, aluminum, which also accumulates in neurofibrillary tangle-containing neurons, stimulates iron-induced lipid peroxidation. (2) Activated microglia, such as those that surround most senile plaques, are a source of NO and O₂ which can react to form peroxynitrite, leaving nitrotyrosine as an. identifiable marker. (3) Amyloid-β itself has been directly implicated in reactive oxygen formation through peptidyl radicals. (4) Advanced glycation end products in the presence of transition metals can undergo redox cycling with consequent reactive oxygen species production.

Hydrogen Peroxide is a reactive oxygen species (ROS) generated in the stereoselective deamination of D-amino acids catalyzed by D-amino acid oxidase enzyme (DAAO, E.C. 1.4.3.3.). Hydrogen peroxide readily crosses cellular membranes and damages DNA, proteins and lipids:

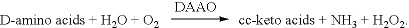

Intra-cellular H₂O₂ generated by DAAO from D-amino acids can be reduced to hydroxyl radicals via transition metals catalyzed Haber-Weiss chemistry. Hydroxyl radical reacts with DNA, lipids and proteins inducing cell death. In a young healthy cell the H₂O₂ produced can be removed by catalase in the peroxisomes or by glutathione peroxidase in the cytosol or plasma membrane. The GSH consumed in the second reaction is regenerated by glutathione reductase using NADPH produced by the oxidative branch of the PPP as reducing equivalents. Inhibition of y-glutamyl cysteine synthetase enzyme can deplete glutathione peroxidase.

As the body ages optical isomers of amino acids very slowly undergo spontaneous, nonenzymatic racemization, so that over a very long period of time an equimolar mixture of the D and L isomers will be formed from the pure L or the pure D isomer. Each L-amino acid racemizes at a known rate at a given temperature. This fact can be used to determine the age of living people and animals or the age of fossil bones. For example, the L-aspartate of the protein dentine present in the outer hard enamel of the teeth, spontaneously racemizes at the rate of 0.10 percent per year at body temperature. Dentine contains only L-aspartate at the time the tooth is formed in childhood. The denture can be isolated from a single tooth of a person and its content of D-aspartate determined. Such analysis has been made on the denture of inhabitants of villages in Ecuador, where individuals claimed to be exceptionally long lived. This test yielded an age of 99 for a woman who was 97 years old by verified records.

At birth all proteins and enzymes are made of 100% L-amino acids. As aging occurs, the rate of D-amino acids present in the proteins and enzymes increase and since D-amino acids do not have biological activity they cause problems for the activity of enzymes and the integrity of structured peptides. The body including the brain neurons contain the enzyme DAAO. This enzyme removes D-amino acids but in doing so produces highly toxic substances i.e. NH₃ and H₂O₂. This production of NH₃ and H₂O₂ is counterbalanced in healthy cells by the production of enzymes such as catalase and glutathione peroxidase, glutathione reductase and the enzyme of glutamylcysteine synthetase.

The enzyme that destroys D-amino acids in the cell are housed in ubiquitous cell organelles called peroxisomes along with a variety of other oxidases which produce H₂O₂ during oxidation of their substrates. Peroxisome also contains catalase and other antioxidant enzymes that assist in the degradation of H₂O₂. The main characteristic of peroxisomes is their inducibility under exposure to certain drugs and xenobiotics. The increase in the number of peroxisomes observed in certain mammalian tissues is accompanied by a heterogeneous enhancement of the different peroxisomal enzyme activities mainly those of the oxidative system. While catalase shows weak induction 2 to 4 fold the oxidative enzymes can be induced by between 20 to 30 fold. This imbalance between the induction of oxyradical producing oxidases and the induction of H₂O₂ scavenging catalase and the glutathione system is the underlying flaw which is exacerbated by the aging organs and the accumulation of D-amino acids in structural peptide and enzymes and ultimately leads to oxidative damage of DNA proteins and lipid peroxidation and which initiates normal degeneration and neoplastic transformation throughout the body. This would explain why several peroxisome proliferators are able to induce hepatocarcinoma in rodents and why chronic exposure to D-amino acids coupled with inhibition of the anti-oxidant enzyme systems of catalase and glutathione leads to neuronal death and the generation of amyloid plaques and dementia together with reduced efficiency in plasma transport metal carrier protein.

As the body ages the proteins and enzymes become more contaminated with D-amino acid groups and their efficiency suffers. This is particularly true of peptides that transport metals and this can lead, as we age, to increased copper and iron deposits in the liver and brain. Clinical features of increasing copper and iron deposits in the liver and brain consist of progressive choreoathetosis, dystoma, dysarthia, dementia, diabetes mellitus and retinal pigmentation. A structurally changed metal transport protein with D-amino acids in its structure may not take up copper from the digestive organs and bind it to serum copper proteins as efficiently and these plasma proteins as they acquire increasing concentrations of D-amino acids in their structure may not oxidize Fe(II) to Fe(III). Many dementia patients initially report with an increased iron uptake in the brain and liver. The iron and copper deposits build up in the liver, pancreas, thyroid gland and in the brain especially. The principle areas of the brain affected are the caudate nucleus, basal ganglia, red nucleus and putamen. For example damage from free radicals has been demonstrated in susceptible neuronal populations in cases of Alzheimer's disease. In this case iron is a potent source of hydroxyl radical generation by the Fenton reaction with H₂O₂. Iron and copper deposits have been associated in many studies with senile plaques and neurofibrillory tangles, which is the pathological hallmark of many dementias or memory impairment conditions. The generation of H₂O₂, which reacts with these metal ions, is from the attempt by the brain's DAAO enzyme to clear the accumulating D-amino acid pool generated in the aging brain. The second breakdown product of DAAO enzyme is ammonia (NH₃) here we have a serious biochemical problem because ammonia is a very toxic substance particularly to the brain. Ammonia is so toxic that even very dilute solutions in the bloodstream can make an animal comatose. The toxicity of ammonia to the brain is not completely understood, but two major factors can be identified.

The pK of ammonia is quite high, so that at the pH of the blood it occurs almost entirely as ammonium ion (NH₄⁺). Ammonium ion cannot readily permeate through the plasma membrane or mitochondrial membranes. However, free ammonia is a neutral molecule and is freely permeant. Although only about 1% of the total ammonia in the blood occurs in the form of free NH₃ at pH 7.4, this small amount can penetrate membranes and gain entry into brain cells and their mitochondria. The entry of ammonia into brain mitochondria leads to the formation of glutamate from ammonia and d-ketoglutarate through the reverse action of glutamate dehydrogenase:

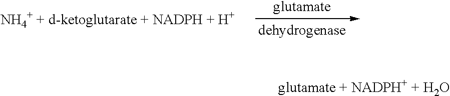

The net result is that cc-ketoglutarate is withdrawn from the pool of citric acid cycle intermediates in brain mitochondria, lowering the rate oxidation of glucose, the major fuel of the brain.

It has been surprisingly found that the compounds of the present invention, particularly oltipraz, are able to remove redox-active transition metals from AD brain sections. Given that there is little in vivo toxicity of the compound when used in a therapeutic setting, these data suggest a certain predication for the abnormally localized iron found in the disease as opposed to a total removal of all cellular iron. In fact, such a notion is supported by our preliminary data showing little/no neurotoxicity in vitro using doses of oltipraz effective at chelating in situ or abolishing amyloid-β toxicity.

The present invention provides a pharmaceutical formulation or a method comprising incorporating the compound in a suitable pharmaceutical carrier, administering a therapeutically or prophylactically effective dosage of the compound incorporated in the carrier to a patient and employing the method in treating a patient for a degenerative disorder or a neurodegenerative disorder, e.g., progressive memory loss.

The present invention also provides a pharmaceutical formulation or a method for therapeutically treating a patient for an illness selected from the group consisting of amnesia, head injuries, Alzheimer's disease, epileptic dementia, presenile dementia, post traumatic dementia, senile dementia, vascular dementia and post stroke dementia. This method may also treat other individuals that seek memory enhancement.

It will be understood by those skilled in the art that the compounds described herein may be used as synergistic agents with neurosteroids and other compounds.

In order to effect the objects of this experiment this invention provides the use of oltipraz of this invention for memory enhancement and a method of using compounds, identified in the embodiments, in a patent for therapeutic and prophylactic purposes.

Parkinson's Disease is a disturbance of voluntary movement in which muscles become stiff and sluggish, movement becomes clumsy and difficult and uncontrollable rhythmic twitching of groups of muscles produces characteristic shaking or tremor. The condition is believed to be caused by a degeneration of pre-synaptic dopaminergic neurons in the brain. The absence of adequate release of the chemical transmitter dopamine during neuronal activity thereby leads to the Parkinsonian symptomatology.

The present invention relates to a pharmaceutical formulation for use in or a method of treatment of disorders of the central nervous system, in particular Parkinson's disease, Huntington's disease, Amylotropic Lateral Sclerosis, by the administration of compounds disclosed herein.

The present invention also provides pharmaceutically acceptable salts of compounds described in the attached embodiments as described herein, or precursors therefore as hereinbefore described, for use in a therapeutic method of treating a warm blooded animal body, for the treatment of indications such as aluminium overload, Alzheimer's disease, Parkinson's disease, Huntington's disease, Amylotropic Lateral Sclerosis, Alexander's disease, malaria, reperfusion injury, cancer and particularly in the treatment of iron overload diseases. The present invention further provides the use of such salts or precursors for the preparation of a pharmaceutical composition for the treatment of the above-mentioned indications, particularly iron-overload diseases.

In accordance with the present invention, a method is provided to treat or prevent a degenerative or related disorder comprising administering to a subject an effective amount of one or more compounds of the present invention.

The present invention also provides the use of one or more of the compounds of the present invention, for the manufacture of a medicament for degenerative or related disorders.

The present invention also provides compounds of the present invention for use in a method of treatment of degenerative or related disorders, said method comprising administering one or more to a subject.

In all of the methods of the instant invention which involve treatment for a particular disorder, the subject to be treated is preferably first identified as being in need of treatment for that disorder, preferably by diagnostic methods known to those skilled in the diagnostic art for that disorder.

In one embodiment, the compounds of the present invention are D-amino acid oxidase inhibitors. By inhibiting D-amino acid oxidase, the production of highly toxic substances i.e. NH₃ and H₂O₂, is greatly reduced. These substances are greatly involved in lipid peroxidation, possibly caused by free radical formation, and perhaps is one of the causative factors of neuronal death.

In another embodiment, the compounds of the present invention enhance phase II detoxification enzymes. The advantage of this is that the effects of neurotoxic agents will be minimized as they are quickly removed. Examples of phase II detoxification enzymes that are enhanced by the compounds of the present invention include: glutathione S transferase (“GST”), γ-glutamylcysteine synthetase (“γ-GCS”), glutathione reductase, glutathione peroxidase, epoxide hydrase, AFB₁ aldehyde reductase, glucuronyl reductase, glucose-6-phosphate dehydrogenase, UDP-glucuronyl transferase, and AND(P)H:quinone oxidoreductase.

In another embodiment, the compounds of the present invention have at least one adjunct residue, the at least one adjunct residue typically bonded to the compounds at R1, R2 R3 or R4.

In another embodiment, the adjunct residue consists of one to eighty amino acids. In another embodiment, the adjunct residue consists essentially of positive charged amino acids. In another embodiment, the adjunct residue consists of one to twenty amino acids of positive charge. In another embodiment, the adjunct residue contains blocks of two or more adjacent amino acids of positive charge. In another embodiment, the positive charged amino acids independently are histidine, arginine or lysine.

In another embodiment, the compounds of the present invention are metal chelating compounds. In a preferred embodiment, the metal chelating compounds specifically chelate iron and/or copper.

In another embodiment, the composition further includes a pharmaceutically acceptable carrier.

In another embodiment, said subject is a neonate and said administering is effected prior to delivery of said neonate and/or during delivery of said neonate.

In another embodiment, the composition is administered enterally, parenterally, topically, orally, rectally, nasally or vaginally. Ophthalmic delivery is also included as an embodiment, e.g., where a compound disclosed herein is administered as a solution to deliver at least a portion of a unit dosage.

In another embodiment, the composition is administered intermittently.

The invention also includes a method for treatment of patients having toxic amounts of metal in the body or in certain body compartments, which comprises administration to the patient, an amount of one or more compounds as described in the attached embodiments to effect reduction of the toxic levels of metal ions in the body of the patient.

The compounds of this invention are useful in the treatment of aluminum intoxication that is found frequently with renal impaired patients, including renal dialysis where aluminum overload in the blood may lead to dialysis encephalopathy.

Even though, compounds in this invention may be of value in treating certain animal pathological conditions, they are especially useful to treat a variety of human conditions. Iron overload conditions associated with beta-thalassemia may be beneficially treated.

In another embodiment, the compounds are selected from the following

[image]

[image]

and oxides, derivatives and metabolites thereof, wherein Z is S, O, NR, R₂ or CR₂; R is —H, —OH, C₁-C₅ alkyl, C₁-C₅ alkoxy or C₁-C₅ alkoxycarbonyl; R₂, together with the atoms to which it is bonded, comprise a spiro ring; R1, R2, R3 and R4 independently are —H, -alkyl, -aryl, -alkylaryl, a heterocycle, a halogen, -alkoxycarbonyl (C₁-C₅) or -carboxyl;

R1, R2, R3 and R4 are each independently H, alkyl, aryl, heterocyclic, halogen, alkoxycarbonyl (C₁-C₅), and carboxyl, wherein said alkyl is defined as C₁-C₁₀ linear or branched chain, saturated or unsaturated which can optionally be singly or multiply substituted by halogen, alkyl (C₁-C₅), hydroxyl, alkoxy (C₁-C₅), alkoxycarbonyl, (C₁-C₅), carboxyl, amido, alkyl amido (C₁-C₅), amino, mono and dialkyl amino (C₁-C₅), alkyl carbamoyl (C₁-C₅), thiol, alkylthio (C₁-C₅), or benzenoid aryl; wherein said aryl is defined as any optionally singly or multiply substituted benzenoid or aryl group (C₆-C₁₄), wherein said heterocyclic is defined as any 4, 5 or 6 membered, optionally substituted heterocyclic ring, saturated or unsaturated, containing 1-3 ring atoms selected from N, O and S, the remaining ring atoms being carbon; and wherein said substituents on said aryl or said heterocyclic are selected from the group consisting of halogen, alkyl (C₁-C₅), hydroxyl, alkoxy (C₁-C₅), alkoxycarbonyl (C₁-C₅), carboxyl, amido, alkyl amido (C₁-C₅), amino, mono and dialkyl amino (C₁-C₅), alkyl carbamoyl (C₁-C₅), thiol, alkylthio (C₁-C₅), benzenoid, aryl, cyano, nitro, haloalkyl (C₁-C₅), alklsulfonyl (C₁-C₅), or sulfonate; where two of said substituents can optionally form part of a fused ring, which can be either saturated, or unsaturated, heterocyclic or carbocyclic or R1 and R2 together or R3 and R4 together independently are oxime (═NOH).

R1, R2 or R3, R4 can form can comprise a spiro ring around the carbon atom(s) to which they are attached or they can form fused or bridged rings to adjacent carbon atoms

Exemplary compounds of the present invention include:

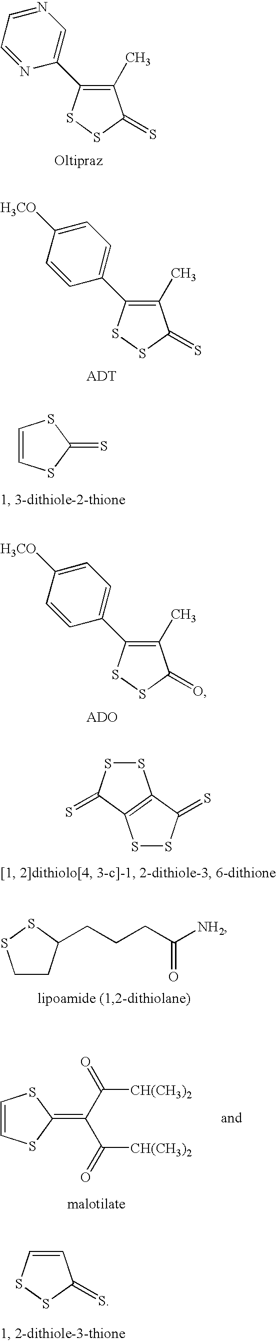

Exemplary derivatives of the present invention include

[image]

[image]

wherein R²⁴ is ═S, ═O, ═N—OH, ═N—R₅, ═N—NH—CO—NH₂, ═N—NH—CS—NH₂, or ═CZZ′; R₅, is C₁-C₆ alkyl or aryl, Z and Z′ independently are —H or an electron-attracting group such as ester or cyano, A is >C═N—OH (oxime), >C═N—OR₃ (where R₃ is hydroxyl, amino, chloro, C_{1,}-C₄, alkoxy, aryl-C_{1,}-C₆ alkyl, a (C₁-C₆ alkyl)carbonyl group or R₃ is an aryl (C₁-C₆ alkyl) carbonyl group) or A is —CHOH, >C═O or >C═N—R₄, where R₄ is C₁-C₆ alkyl or aryl group; Y2 is an acceptable or nontoxic anion, e.g., as disclosed herein (e.g., Cl⁻, Br⁻, I⁻ or OH⁻).

R₁ and R₂ independently are hydrogen, a halogen, nitro, nitroso, a thiocyano group, a C₁-C₆ alkyl group, a C₂-C₆ alkenyl group, an aryl group, aryl (C₁-C₆ alkyl) group, an aryl (C₂-C₆ alkenyl) group, a carboxyl group, a (C₁-C₆ alkyl) carbonyl group, an aryl carbonyl group, a (C₁-C₆ alkoxy)carbonyl group, a (C₁-C₆ alkoxy)carbonyl (C_{1,}-C₆ alkyl) group, a C₁-C₆ alkoxy group, a trifluoromethyl group, an amino group, a di(C₁-C₆ alkyl) amino(C₁-C₆ alkyl) group, an acylamino group of formula —NHCOCₙH₂ₙ₊₁ with n from 0 to 6, a group —NH—CSCₙH₂ₙ₊₁ with n from 0 to 6, a terpenyl group, a cyano group, a C₂-C₆ alkynyl group, a C₂-C₆ alkynyl group substituted with a C₁-C₆, alkyl or an aryl group, a hydroxy(C₁-C₆ alkyl) group, a (C₁-C₆ acyl) oxy (C₁-C₆ alkyl) group, a (C₁-C₆ alkyl) thio group and an arylthio group, or

R₁ and R₂ together comprise a mono- or polycyclic C₂-C₂₀ alkylene group optionally comprising one or more hetero atoms, with the exception of the 2,2-dimethyltrimethylene group, or a C₃-C₁₂ cycloalkylene group;

R is C₁-C₆, alkyl,

R²⁰ independently is —SH, —SCH₃, —S(O)CH₃, —OH, —OCH₃, —S—C1-C6 alkyl opotionally substituted with 1, 2 or more independently selected —O—, —S—, —OH, halogen, —CN, ═O or —C(O)—NH— moieties, or R²⁰ independently is —S—C1-C6 alkyl opotionally substituted with 1, 2 or more independently selected —O—, —S—, —OH, halogen, —CN, ═O or —C(O)—NH— moieties (typically R20 independently is —SH, —S(O)CH₃ or —SCH₃);

R²¹ is C1-C6 alkyl, typically methyl; and

R²² is ═O or ═S;

and the pharmaceutically acceptable salts of any of these compounds.

In the foregoing definition, the aryl moiety of an arylalkyl group means an optionally substituted aromatic carbon-based group such as a phenyl or naphthyl group or an optionally substituted aromatic heterocyclic group such as a thienyl of furyl group, with 1, 2, 3 or more substituents optionally selected from halogen, C₁-C₄ alkyl, C₁-C₄ alkoxy, trifluoromethyl, nitro and hydroxyl.

Alternatively, another group of compounds is formed when A (FIGS. 10 & 11) is a group C═N—OR′₃where R′₃ is an optionally substituted C₁-C₆ alkyl group, in particular substituted with one, two or more groups independently chosen from hydroxyl, amino, chloro, bromo, fluro, iodo and C₁-C₄ alkoxy groups, or an aryl (C₁-C₆ alkyl) group.

Exemplary compounds include compounds of formula

[image]

where R₃ has the meaning given above.

Another group of compounds is formed when A (FIGS. 10 & 11) is a group C═N—O—CO—R″₃, R″₃ being chosen from a hydrogen atom, an optionally substituted C₁-C₆ alkyl group, an aryl group and an aryl (C₁-C₆ alkyl) group, i.e.,

[image]

Alternatively other groups of compounds are formed when A (FIGS. 10 & 11) is a CH—OH group, i.e.,

[image]

or when A (FIGS. 10 & 11) is a group C═N—R, R, being a C₁-C₆ alkyl or an aryl group, i.e.,

[image]

or when A (FIGS. 10 & 11) is a C(O) group and R²⁴ is an oxygen atom, i.e.,

[image]

wherein R₁ and R₂ independently are —H, a halogen, nitro, nitroso, thiocyano, C₁-C₆ alkyl, C₂-C₆ alkenyl, aryl, aryl (C₁-C₆ alkyl), aryl (C₂-C₆ alkenyl), carboxyl, (C₁-C₆ alkyl) carbonyl, arylcarbonyl, (C₁-C₆ alkoxy)carbonyl, (C₁-C₆ alkoxy) carbonyl (C₁-C₆ alkyl), C₁-C₆ alkoxy, trifluoromethyl, amino, di-(C₁-C₆ alkyl)amino, (C₁-C₆, alkyl), acylamino of formula —NHCOCₙH₂ₙ₊₁ (n is 0, 1, 2, 3, 4, 5 or 6), a group —NH—CSCₙH₂ₙ₊₁ (n is 0, 1, 2, 3, 4, 5 or 6), terpenyl, cyano, C₂-C₆ alkynyl optionally substituted with C₁-C₆ alkyl or aryl, or R₁ is a —OH or C₁-C₆ alkyl, a (C₁-C₆ acyl)-oxy(C₁-C₆ alkyl), (C₁-C₆ alkyl)thio or arylthio, but R₂ is typically not —H,

or R₁ and R₂ together comprise a mono- or polycyclic C₂-C₂₀ alkylene group optionally comprising one or more independently selected O, N or S atoms. In some embodiments, R₂ is C₁-C₆ alkyl, C₂-C₆ alkenyl, aryl, aryl(C₁-C₆ alkyl), aryl C₂-C₆ alkenyl, terpenyl, C₂-C₆ alkynyl optionally substituted with C₁-C₆ alkyl or aryl. In some embodiments, R is chosen from C₁-C₆ alkyl.

Exemplary oximes of derivatives of the present invention include:

[image]

Additionally aldehydes or ketones of previously identified compounds are included as shown in FIG. 15

[image]

Exemplary 1,2-dithiol-3thione derivatives have a formula shown in FIG. 28

[image]

wherein R is —H, halogen, lower alkoxy, amino, lower alkyl optionally substituted with amino or lower alkoxy carbonyl, wherein the term “lower” means methyl, ethyl, propyl and butyl, including structural isomers such as isopropyl, isobutyl and tertiarybutyl.

Among the compounds of the formula shown in FIG. 28, preferred compounds include 5-(4-phenyl-1,3-butadienyl)-1,2-dithiol-3-thione, 5-4(4-chlorophenyl)-1,3-butadienyl-1,2-dithiol-3-thione, 5-{4-(4-methoxyphenyl)-1,3-butadienyl}-1,2-dithiol-3-thione, 5-{4-(p-toluyl)-1,3-butadienyl}-1,2-dithiol-3-thione, 5-{4-(o-chlorophenyl)-1,3-butadienyl}-1,2-dithol-3-thione and 5-{4-(m-methyl phenyl)-1,3-butadienyl}-1,2-dithiol-3-thione.

Another exemplary 1,2-dithiole is:

[image]

wherein Het is pyrimidin-2-yl, pyrimidin-4-yl, or pyrimidin-5-yl, which are optionally substituted by one, two or more independently selected halogen, C1-4 alkyl, C1-4 alkoxy, mecapto, C1-4 alkylthio, or di-C1-4-alkyl-amino, C1-4-alkoxy-carbonyl, carboxy, C1-4-alkoxy-carbonyl, carbamoyl, C1-4-N-alkyl-carbamoyl, or R,—CH(OH)— in which R, represents hydrogen or alkyl of 1 through 3 carbon atoms.

Exemplary 1,2-dithiole compounds include 4-ethyl-5-(pyrimidin-5-yl)-1,2-dithiole-3-thione, 4-methyl-5-(5-methylthiopyrimidin-4-yl)-1,2-dithole-3-thione and 5-(5-chloropyrimidin-4-yl)-4-methyl-1,2-dithole-3-thione.

Exemplary 1,2-dithiol-3-thione-S-oxides have the following formula:

[image]

wherein R₁ is C1-4 alkyl, lower alkoxy, hydroxy, halogen, trifluoromethyl or nitro, and R₂ represents hydrogen, halogen or lower alkoxy, or R₁ and R₂ are bonded to adjacent carbon atoms and together form an alkylene dioxy group with 1-2 carbon atoms. These compounds include ones where R₁ is fluorine, chlorine, bromine, iodine or methoxy, and R₂ is hydrogen.

An exemplary 1,3-dithiolo-(4,5-d)-1,3-(dithiio-2-thione) compound corresponds to the formula:

[image]

wherein R′ is —H. —Br, —Cl, —F, I, —CN or —CH₂(CH₂)ₙCH₃ and n is an integer of from 0 to 14. Exemplary compounds are 1,3-dithiolo(4.5-d)-1,3-(dithiino-2-thion 1,3-dithiolo(4.5-d)-1,3-dithole-2-thione; 5-chloro-1,3-dithiolo (4.5-d)-1,3-dithiole-2-thione; and 5-cyano-13-dithiolo(4.5-d)-1,3-dithiole-2-thione.

Exemplary 1,3-dithiole derivatives have the formula:

[image]

wherein R₁ and R₂ together form alkylene or alkenylene having from 3 to 6 carbon atoms, or O, S or N, any of which may have a substituent selected from the group consisting of lower alkyl, lower alkenyl, lower alkynyl, lower alkoxycarbonyl, hydroxy-substituted lower alkyl, aryland aralkyl, and said alkylene or alkenyiene substituted by one or two substituents selected from the group consisting of lower alkyl, carboxyl, lower alkoxycarbonyl, and —C(O)—NR^{c}R^{d} wherein each of R^{c} and R^{d} independently is —H, lower alkyl, aryl or heteroaryl, provided that at least one substituent on the alkylene or alkenylene group is carboxyl, loweralkoxycarbonyl or —C(O)—NR^{c}R^{d}, and Q is an acid residue.

Another group of compounds is formed in which R¹ and R² together form —(CH₂)₄—, —(CH₂)₅—, ——(CH₂)₆—, —CH₂OCH₂CH₂—, —CH₂SCH₂CH₂—, —CH₂CH₂SCH₂CH₂CH₂—, —CH₂CH₂OCH₂CH₂—, —CH₂CH₂NHCH₂CH₂—, —CH₂CH₂N(ph)₂CH₂CH₂—, —CH₂CH₂N(CH₂ph)CH₂CH₂—, —CH₂CH₂(CH₃) CH₂CH₂—, —CH₂CH═CHC—H₂—CH₂CH═CHCH₂CH₂—, which may be substituted by carboxyl, methyl, ethyl, n-propyl, isopropyl, n-butyl, n-pentyl, n-hexyl, methoxycarbonyl, ethoxycarbonyl, isopropoxycarbonyl, carbamoyl, N-methylcarbamoyl, N,N-dimethylcarbamoyl, N-phenylcarbamoyl or N-benzylcarbamoyl, and Q is an acid residue of hydrochloric acid, hydrobromic acid, hydroiodic acid, nitric acid, perchlonc acid, borofluoric acid, sulfuric acid, phosphoric acid, oxalic acid, tartaric acid, citric acid, methanesulfonic acid or p-toluenesulfonic acid.

Other exemplary compounds are those where R¹ and R² comprise 2-ethoxycarbonylpyrrolidinium, 2-carboxypyrrolidinium, 2-carbamoylpyrrolidinium, 4-ethoxycarbonylthiazolidinium, 2-ethoxycarbonylpiperidinium, 3-ethoxycarbonylpiperidirtium, 4-ethoxycarbonylpiperidinium, 4-carboxypiperidinium, 4-carbamoylpijperidinium, 3-ethoxycarbonyl-6-methyl piperidinium or 4-ethoxycarbonylpiperazinium, and Q is ClO₄, Cl, Br, I or HSO₄.

Exemplary 1,2-dithiol-3-ylideneammonium derivatives have the formula:

[image]

wherein Xθ (or X⁻) is a pharmaceutically acceptable anion, R is a straight- or branched-chain alkyl radical containing 1 to 7 carbon atoms [unsubstituted or substituted by a hydroxy, carboxy, alkoxycarbonyl, cyano, dialkylamino or alkylcarbonyl radical, or a benzoyl radical the phenyl ring of which is unsubstituted or substituted by one or more halogen atoms or radicals selected from alkyl (optionally substituted by one or more halogen atoms), alkoxy, hydroxy, amino, alkylamino, dialkylarnino, cyano, and nitro, or by a thenoyl radical the thienyl ring of which, is unsubstituted or substituted by one or more halogen atoms or radicals selected from alkyl, cyano and nitro, or a pyridylcarbonyl, carbamoyl, dialkylcarbamoyl (the alkyl radicals of which can together form, with the nitrogen atom to which they are attached, a 5- or 6-membered heterocyclic ring optionally containing another heteroatom selected from oxygen, sulphur, and nitrogen substituted by an alkyl or alkylcarbonyl radical) or pyridy) radical], a dialkylcarbamoyl radical (the alkyl radicals of which can together form. with the nitrogen atom to which they are attached, a 5- or 6-membered heterocyclic ring optionally containing another heteroatom selected from oxygen, sulphur, and nitrogen substituted by an alkyl or alkycarbonyl radical), an alkenyl radical containing 2 to 6 carbon atoms, an alkynyl radical containing 2 to 6 carbon atoms, or an alkoxycarbonyl radical, or alternatively represents a 2-oxotetrahydrofuran-3-yl or 2 oxotetrahydropyran-3-yl ring, and either R₁ and R₂ which have the same or different significances each represent a phenyl radical, a cycloalkyl radical containing 3 to 7 carbon atoms, or an alkyl or phenylakyl radical, or alternatively together form, with the nitrogen atom to which they are attached a 5-, 6-or 7-membered heterocylic ring which can optionally contain another hetero-atom selected from oxygen sulphur, and nitrogen substituted by an alalkyl radical, or R₁ represents a phenyl radical unsubstituted or unsubstituted by one or more halogen atoms or radicals selected from alkyl (optionally substituted by one or more halogen atoms), alkoxy, hydroxy, amino alkylamino, dialkylamino, cyano and nitro, or alternatively represents a cycloalkyl radical containing 3 to 7 carbon atoms, or an alkyl or phenylalkyl radical, and R₂ represents a hydrogen atom, and also the corresponding bases when R₂ represents-a hydrogen atom, the aforementioned alkyl and alkoxy radicals and moieties containing 1 to 4 carbon atoms in a straight- or branched-chain unless otherwise indicated.

In other embodiments, Xθ is a pharmaceutically acceptable anion, R is a straight- or branched-chain alkyl radical containing 1 to 7 carbon atoms [unsubstituted-or substituted by hydroxy, carboxy, alkoxycarbonyl, cyano, dialkylamino, alkylcarbonyl, benzoyl, thenoyl, pyridyl, carbonyl, carbamoyl, dialkylcarbamoyl (the alkyl radicals of which can together form, with the nitrogen atom to which they are attached, a 5- or 6-membered heterocyclic ring optionally containing another hetero-atom selected from oxygen, sulphur, and nitrogen substituted by an alkyl or alkylcarbonyl radical) or pyridyl radical], a dialkylcarbamoyl radical (the alkyl radicals of which can together form, with the nitrogen atom to which they are attached, a 5- or 6-membered heterocyclic ring optionally containing another hetero-atom selected from oxygen, sulphur, and nitrogen substituted by an alkyl or alkylcarbonyl radical), an alkenyl radical containing 2 to 6 carbon atoms or an alkynyl radical containing 2 to 6 carbon atoms, and either R₁ and R₂, which have the same or different significances, each represent a phenyl radical, a cycoalkyl radical containing 3 to 7 carbon atoms, or an alkyl or phenylalkyl radical or alternatively together form, with the nitrogen atom to which they are attached, a 5-, 6- or 7-membered heterocyclic ring which can optionally contain another hetero-atom selected from oxygen, sulphur, and nitrogen substituted by an alkyl radical, or R₁ represents a phenyl radical a cycloalkyl radical containing 3 to 7 carbon atoms, or an 1 alkyl or phenylalkyl radical, and R₂ represents a hydrogen atom, and also the corresponding bases when R₂ represents hydrogen, the aforementioned alkyl and alkoxy radicals and moieties containing 1 to 4 carbon atoms in a straight or branched-chain unless otherwise mentioned.

In other embodiments, Xθ is a pharmaceutically acceptable anion, R represents an alkenyl radical containing 2 to 6 carbon atoms, or a straight- or branched-chain alkyl radical containing 1 to 7 carbon atoms [unsubstituted or substituted by a cyano, dialkylamino, carbamoyl, alkylcarbonyl or thenoyl radical, or a benzoyl radical the phenyl ring of which is unsubstituted or substituted by one or more halogen atoms or radicals selected from alkyl, alkoxy, hydroxy and, cyanol, the aforementioned alkyl and alkoxy radicals and moieties containing 1 to 4 carbon atoms in a straight- or branched-chain unless otherwise stated, and R₁ and R₂ together-with the nitrogen atom to which they are attached represent a pyrrolidin-1-yl or morpholino radical.

In other embodiments, Xθ is a pharmaceutically acceptable anion, R represents a methyl or ethyl radical unsubstituted or substituted by a benzoyl radical the phenyl ring of which is unsubstituted or substituted by one or more halogen atoms or radicals selected from alkyl and alkoxy radicals containing 1 to 4 carbon atoms in a straight- or branched-chain, and the hydroxy and cyano radical and R₁ and R₂ together with the nitrogen atom to which they are attached are a morpholino radical.

Exemplary 1,2-dithio-1-3-ylideneammonium derivatives include:

N-[5-(4-chlorophenacylthio)-1 2-dithiol-3-ylidene] morpholinflum chloride;

N-[5-(3-methoxyphenacylthio)-1,2-dithiol-3-ylidene]-morpholinium chloride;

N-[5-(4-fluorophenacylthio)-1,2-dithiol-3-ylidene]-morpholinium chloride;

N-[5-(2,4-dichlorophenacylthio)-1,2-dithiol-3-ylidene]-morpholinium chloride;

N-[5-(2-chlorophenacylthio)-1,2-ditliiol-3-ylidene]-morpholinium iodide;

N-[5-(4-hydroxyphenacylthio)-1,2-dithiol-3-ylidene]-morpholinium chloride;

N-[5-(4-methoxyphenacylthio)-1,2-dithiol-3-yllidene]-morpholinium iodide;

N-[5-(4-methylphenacylthio)-1,2-dithiol-.3-ylidene]-morpholinium chloride;

N-[5-(4-cyanophenacylthi,-. o)-1,2-dithiol-3-ylidene]-morpholinium chloride; and

N-[5-(phenacylthio)-1,2dithiol-3-ylideene]-morpholinium chloride.

Exemplary isobenzothiazolone derivatives of the present invention further include:

[image]

wherein at least one of R¹ and R² is preferably nitro, arylazo, substituted arylazo, benzylidenearnino or substituted benzylidenearnino. When only one of R¹ and R² is so substituted, one of R¹ and R² may be hydrogen. The R³ substituent is selected from alkyl groups in less than about 7 carbon atoms, amino, hydroxyl, alkoxyl, and aryl groups (and functionalized forms thereon,)

Preferred species of the isobenzothiazole derivative of the present invention comprise R¹ as nitro or arylazo and R² as hydrogen, for example. Examples include compounds where R² is hydrogen and R¹ is phenylazo; substituted arylazo such as 4-hydroxyphenylazo; 4-nitro-2-methylphenylazo; 2-hydroxy-l-napthylazo; 2-hydroxy-5-methylphenylazo; 2-hydroxy4-methyl-5-nitrophenylazo;

4-hydroxy-l-napthylazo; 4-hydroxy-3-methyl-1-napthylazo; 4-hydroxy-5-aza-1-napthylazo; 2-amino-l-napthylazo; 1-hydroxy-2-napthylazo; 3-N,N-dimethylaminopropylcarboxyamido-l-hydroxy-4-naph-thylazo; 1-hydroxy-4-methoxy-2-naphthylazo, 2 hydroxy-3-carboxy-l-naphthylazo; 1-hydroxy-3,6disulfonato-2-naphthylazo; 2,3-dihydroxy-l-naphthylazo; or 2-hydroxy-3,5-dimethyl-l-phenylazo. In one particular embodiment R¹ is the substituted ben zylideneamino, 2,4-dinitrobenzylideneamino and R²is hydrogen. Additionally R¹ as hydrogen and R² as 2-hydroxy-l-naphthylazo or 4-hydroxy-lphenylazo.

In one aspect, R³ substituents with sufficient polarity to confer aqueous solubility upon the compound. For example, R³ may be —(CH₂)nR⁴R⁵ where n is from 2 to 6 and R⁴ and R⁵ are simple alkyls or hydrogens. Other possible water solubilizing side chains include 3-carboxypropyl, sulfonatoethyl and polyethyl ethers of the type —CH₂(C—H₂OCH₂),CH₃ where n is less than 10. Preferred compounds include R³ side chains containing aminoalkyl, carboxyalkyl, omega amino polyethyl ethers and N-haloacetyl derivatives. In a broader sense, for various utilities R³ may be alkyl, aryl, heteroaryl, alkoxy, hydroxy or amino groups. When including substitutions for solubility or reactivity purposes, R³ may be aminoalkyl, carboxyalkyl, hydroxyalkyl or haloalkyl. The aryl or heteroar R³ moieties may be substituted, for example as aminoaryl, carboxyryl or hydroxyaryl.

Alternatively the isobenzothiazolone derivatives can have the following structure:

[image]

wherein at least one of R¹ and R² is nitro, arylazo, substituted arylazo, benzylideneamino or substituted benzyfideneamino and one of R¹ and R² may be hydrogen and R³ is a aminoallayl, aminoaryl and aminoheteroaryl, carboxyalkyl, carboxyaryl or carboxyheteroaryl covalently linked to a polymer comprising amino or hydroxy groups. The spacer arm R³ can comprise oligmers or polyethylene-glycol and its derivatives. In one aspect, R³ may be 17-chloracetamido-3,6,9,12, 1 5-pentaoxyheptadecyl where hexaethylene glycol has been chloroacetamidated. When the polymer groups, Y¹ and R³ comprises carboxyl groups, the covalent linkage is preferably through an ester bond. When the polymer comprises amino groups, the analog covalent linkage is through an amide bond. The amine bearing polymer, when coupled to R³, may be a polymer such as chitosan, polyalkylamine, aminodextran, polyethyleneimine, polylysine or amityrene.

The R³ substituents of the present invention may also comprise an alkyl linked to an amine bearing polymer by amine displacement of a halogen from an alpha-haloalkyl or alpha-haloalkylcarbox amido R³ precursor. In the case of aminoalkyl or aminoaryl groups the R³ substituent may also be covalently linked to a polymer such as polyepichlorohydrin, chloromethylpolystyrene, polyvinyl alcohol or polyvinyl pyridine. The R³ substituent of the present invention may generally be an aminoalkyl, hydroxyalkyl, aminoaryl or hydroxyary] group linked to a polymer comprising carboxyl groups through amide or ester linkages.

When polymers are involved in the R³ structure, the polymer may be one such as polyacrylic acid, polymethacrylic acid, polyitaconic acid, oxidized polyethylene oxide, poly(methylmethacrylate/methacrylic acid), carboxymethyl cellulose, carboxymethyl agarose or carboxymethyl dextran. When such a carboxyl polymer is involved, the R³ may be aminoalkyl (such as 8 aminohexyl, for example), hydroxyalkyl, aminoaryl or hydroxyaryl linked to the polymer through amide or ester linkages. In such cases, an R³ precursor function may bear an amine or hydroxyl group to be covalently linked to a polymer by reaction with an acid anhydridebearing polymer or by coupling with a carboxylate bearing polymer through carbodimide induced bond formation.

The R³ substituent or precursor thereto may also be a haloalkyl or carboxylialoalkyl moiety such as chloracetamido. Such a substituent may readily be coupled to an amine bearing polymer by amine displacement of the halogen.

The compounds of the present invention include

[image]

wherein R₁ and R₂ independently are hydrogen, halogen, nitro, nitroso, thiocyano, C₁-C₆ alkyl, C₂-C₆ alkenyl, aryl, aryl-C₁-C₆ alkyl, an aryl (C₂-C₆ alkenyl) group, carboxyl, C1-C₆ alkyl-carbonyl, arylcarbonyl, (C₁-C₆ alkoxy)carbonyl, (C₁-C₆ alkoxy)carbonyl (C_{1,}-C₆ alkyl), C₁-C₆ alkoxy, trifluoromethyl, amino, di-(C₁-C₆ alkyl)-amino(C₁-C₆ alkyl), a acylamino group of formula —NHCOCₙH₂ₙ₊₁ with n from 0 to 6, —NHCSCₙH₂ₙ₊₁ (where n is 0, 1, 2, 3, 4, 5 or 6), a terpenyl group, cyano, C₂-C₆ alkynyl (optionally substituted with C₁-C₆, alkyl or aryl), a hydroxy(C₁-C₆ alkyl) group, a (C₁-C₆ acyl) oxy (C₁-C₆ alkyl) group, a (C₁-C₆ alkyl) thio group and arylthio, or R₁ and R₂ together form a mono- or polycyclic C₂-C₂₀ alkylene group optionally comprising one or more O, N or S atoms or the pharmaceutically acceptable salts of these compounds.

In the foregoing definition, aryl group or aryl fraction of an arylalkyl group denotes an aromatic carbon-based group such as a phenyl or naphthyl group or an aromatic heterocyclic group such as a thienyl of furyl group. It is possible for these groups to bear one or more substituents chosen from a halogen atom, C₁-C₄ alkyl, C₁-C₄ alkoxy, trifluoromethyl, nitro and hydroxyl.

Other compounds suitable for use in the invention methods include

[image]

wherein R₁ and R₂ are independently (═O) or —OR, where R is H or (C₁-C₄) alkyl; and R₃ is H or (C₁-C₄) alkyl. Preferably, R₃ is H. Preferably R₁ and R₂ are (═O) or OH and

[image]

wherein X is H or both Xs represent a direct bond between the two sulfur atoms; R₁ is (═O) or —OH; and R₂ is H, Na, K or (C₁-C₄)alkyl, in particular the compound maybe 3-keto lipoic acid, 3-hydroxy lipoic acid, 3-keto dihydrolipoic acid or 3-hydroxy dihydrolipoic acid.

The compounds of the present invention can be further selected from the group comprising:

[image]

Other exemplary compounds include:

[image]

wherein Y is selected from nitro and trifluoromethyl; X is selected from alkyl and alkenyl of up to 6 carbon atoms, nitro, trichloromethyl, trifluoromethyl, trifluoromethoxy, trifluoromethylthio, trifluoromethylsulfoxyl, trifluoromethylsulfonyl, methoxymethyl, cyano, carboxy, halogen (F, Cl, Br, I), hydroxy, acetylamino, amino, N-phenylamino, N,N-diallylamino, alkoxy, N-morpholino, N-piperidino, N-piperazino, N-pyrrolidino, dimethylaminodithiocarbarnyl, carboalkoxy, alkylthio, mono- and dialkylarnino, N-alkyl-carbamyl, N,N-dialkylcarbarnyl, alkylsulfoxy, alkylsulfonyl, said alkyl groups containing from 1 to 4 carbon atoms; n is an integer from 1 to 3 wherein at least one of said X groups is selected from N-morpholino, N-piperidino, N-piperazino or N-pyrrolidino; and salts thereof. Other compounds of this type include those where (1) Y is nitro and n is 1, (2)

Y is trifluoromethyl and n is 1, (3) Y is trifluoromethyl. and n is 2, (4) Y is nitro and n is 2, (5) Y is CF₃ and n is 1, (6) Y is CF₃ and n is 3.

Another exemplary compound suitable for invention methods is S-tertbutyl-S′-(2,4-dinitro-3-aminopropyl-6-tri-fluoromethylphenvl)-trithiocarbonate.

Other water soluble exemplary compounds of the present invention include:

[image]

wherein R is H or a C₁ to C₁₂ alkyl moiety; R₁ is a C₆ to C₁₂ arylene moiety; R₂ is a C₁ to C₄ alkylene moiety and n is 2 to 50 and

[image]

wherein the dotted line is an optionally present double bond and wherein the groups R₁ and R₂ are independently hydrogen, C₁₋₂₀ alkyl or C₂₋₁₂ alkenyl, C₁₋₄ alkoxy or C₂₋₄ alkeny, including compounds where R₁ and R₂ are both hydrogen.

Other exemplary compounds suitable for use in the invention methods have the formula

[image]

wherein R and R1 independently are C1-12 alkyl or C5-12 cycloalkyl optionally substituted with 1, 2 or more C1-4 alkyl or C7-14 aralkyl, and Y is hydrogen, mercapto or SR′ where R′ is C1-20 alkyl (including C618 alkyl), C5-12 cycloalkyl, C3-20 alkenyl, or C7-14 aralkyl. For other compounds, R and R1 independently are C3-8 branched-chain alkyl, 1-methylcyclohexyl or dimethyl benzyl. Exemplary compounds are 4-(3,5-di-isopropyl-4-hydroxyphenyl)-1,2-dithole-3-thione; 4-(3,5-di-t-butyl-4-hydroxyphenyl)-1,2-dithiole-3-thione, 4-[3,5-bis(l,l-dimethylpropyl)-4-hydroxyphenyl]-1,2-dithi-ole-3-thione, 4-[3,5-bis(l,l-dimethylbutyl)-4-hydroxyphenyl]-1,2-dithiole-3-thione, 4-[3,5-bis(1,1,3,3-tetramethylbutyl)-4-hydroxyphenyl]-1,2-dithiole-3-thione, 4-[3,5-bis(l-methylcyclohexyl)-4-hydroxyphenyl]-1,2-dithiole-3-thione, 4-[3,5-bis(l,l-dimethylbenzyl)-4-hydroxyphenyl]-1,2-dithiole-3-thione, 4-(3t-butyl-4-hydroxy-S-isopropylphenyl)-1,2-dithiole-3-thione, 4-(3-t-butyl-4-hydroxy-5-methylphenyl)-1,2-dithiole-3-thione, 4-[3-(1,1-dimethylpropyl)-4-hydroxy-5-isopropylphenyl]-1,2-dithiole-3-thione, 4-[3-(1,1-dimethylbenzyl)-4-hydroxy-5-isopropylphenyl]-1,2-dithole-3-thione, 5-benzylthio-4-(3,5-di-t-butyl-4-hydroxyphenyl)-1,2-dithole-3-thione, 5-benzylthio-4-[3,5-bis(l,l-dimethylpropyl)-4-hydroxy-phenyl]-1,2-dithiole-3-thione, 5-hexylthio-4-(3,5-di-t-butyl-4-hydroxyphenyl)-1,2-dithiole-3-thione, 5-hexylthio-4-[3,5-bis(l,l-dimethylbutyl)-4-hydroxy-phenyl]-1,2-dithiole-3-thione, 5-octadecylthio-4-(3,5-di-t-butyl-4-hydroxyphenyl)-1,2-dithiole-3-thione, 5-octadecylthio-4-[3,5-bis(l,l-dimethylbenzyl)-4-hydroxyphenyl]-1,2-dithiole-3-thione, 5-allylthio-4-(3,5-di-t-butyl-4-hydroxyphenyl)-1,2-dithiole-3-thione, 5-cyclohexylthio-4-(3,5-di-t-butyl-4-hydroxyphenyl)-1,2-dithole-3-thione and 4-(3,5-di-sec-butyl-4-hydroxyphenyl)-1,2-dithiole-3-thione. In related compounds Y is

[image]

which is bonded through a hydrogen atom of the FIG. 41 structure.

Other compounds suitable for use in invention methods the formula

[image]

wherein A is —CH₂— or —O—, R¹ and R² independently are —H, ═OH, a halogen, lower alkyl or lower alkoxy, and n is 0, 1, 2 or 3 when A is —CH₂—, or 1, 2 or 3 when A —O— or a salt of these compounds. In some embodiments, A is —CH₂— and R² is —H, or a salt thereof. In other embodiments, R¹ is —H, —OH or lower alkoxy, or a salt thereof. In yet other embodiments, A is —O— and R²—H, or a salt thereof or R, is —H, —OH or lower alkoxy or a salt thereof.

Examples include the following compounds (a) through (k) and their salts

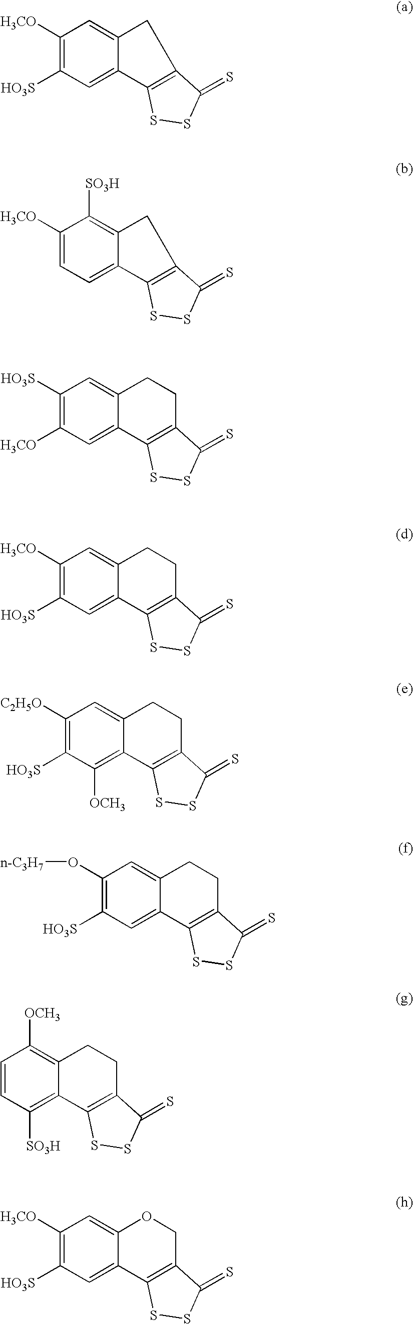

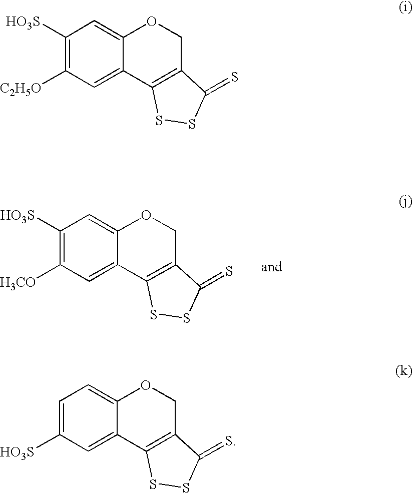

Other exemplary compounds include

[image]

wherein k is 0, 1, 2, 3, 4 or 5, X and Y independently are —H, lower alkyl or lower alkoxy and R¹¹ is alkyl or —(CH₂)ₘ—C₆H₂—(R12)(R13)(R14) wherein m is 0, 1, 2,3 or 4 and R12, R13 and R14 are independently —H, lower alkyl or lower alkoxy, or a salt thereof. Optionally excluded are compounds where k and m are zero, —SO₃H is bonded to the 3-position, X is 4-methoxy, and R12, R13, R14, and Y are —H. In some embodiments, R¹¹ is alkyl or R¹¹ is —(CH₂)ₘ—C₆H₂—(R12)(R13)(R14) and the sulfo group bonds to the 3-position, X is a 4-methoxy group, and R12, R13, R14 and Y are each a hydrogen atom); or a salt thereof.

Examples include any of the following compounds or a salt thereof 5-hexyl-4-(4-methoxy-3-sulfobenzyl)-3H-1,2-dithiole-3-thione and 4-(4-methoxy-3-sulfophenyl)-5-(p-toluyl)-3H-1,2-dithiole-3-thione.

As used herein, the compounds that are named or shown by chemical structures herein are sometimes referred to as “compounds of the present invention”, “compounds of the invention” or they are referred to using similar terms. These compounds are suitable for use in treating the conditions or diseases disclosed herein or they are suitable for ameliorating one or more symptoms associated with any of these conditions or slowing the progression of the conditions.

According to the present invention the degenerative and related disorders include Parkinson's disease, Huntington's disease, Amylotrophic Lateral Sclerosis, Cerebral amyloid angiopathy, Multiple Sclerosis, cognitive disorders, Progeria, Alzheimer's disease, epileptic dementia, presenile dementia, post traumatic dementia, senile dementia, vascular dementia, HIV-1-associated dementia, post-stroke dementia, Down's syndrome, motor neuron disease, amyloidosis, amyloid associated with type 11 diabetes, Creutzfelt-Jakob disease, necrotic cell death, Gerstmann-Straussler syndrome, kuru and animal scrapie, amyloid associated with long-term hemodialysis, senile cardiac amyloid and Familial Amyloidotic Polyneuropathy, cerebropathy, neurospanchnic disorders, memory loss, aluminum intoxication, reperfusion injury, reducing the level of iron in the cells of living subjects, reducing free transition metal ion levels in mammals, patients having toxic amounts of metal in the body or in certain body compartments, and related degenerative disorders.

In another embodiment, the degenerative disorders are neurodegenerative disorders and can be collected from the group comprising: Parkinson's disease, Alzheimer's disease, Huntington's disease, Amylotropic Lateral Sclerosis, epileptic dementia, presenile dementia, post traumatic dementia, senile dementia, vascular dementia and post stroke dementia, Down's syndrome, and Creutzfelt-Jakob disease.

In accordance with the present invention, a method is provided to treat or prevent malaria or a trypanosome infection comprising administering to a subject having the infection or at risk thereof an effective amount of one or more compounds of the present invention.

The present invention also provides the use of one or more of the compounds of the present invention, for the manufacture of a medicament for treating or preventing any of the conditions or diseases disclosed herein or for amelioration of a symptom(s) associated therewith or for slowing the progression or worsening of the disease or its symptom(s).

Also provided is a pharmaceutical formulation comprising a pharmaceutically acceptable carrier and at least one compound of the invention. These formulations include unit dosage forms, e.g., tablets, capsules, and the like. The formulations are suitable for treating or preventing any of the conditions or diseases disclosed herein (or ameliorating one or more symptoms thereof).

The present invention also provides a method for reducing the level of iron in the cells of living subjects by administering a pharmaceutical formulation comprising one or more of the compounds of the present invention.

In another embodiment the compounds of the present invention are micronised.

In another embodiment the compounds of the present invention are administered in an ophthalmic solution and are preferably in a pharmaceutical formulation that further includes an anti microbial preservative.

In another embodiment the compounds of the present invention are administered to a mammal and functions as a chelating agent specifically for Iron and/or Copper.

In another embodiment the compounds of the present invention are formulated in a pharmaceutical formulation, which further includes phosphatidyl-choline or di-phosphatidyl-choline.

In another embodiment the compounds of the present invention are complexed with phosphatidyl-choline or di-phosphatidyl-choline in a pharmaceutical formulation.

In another embodiment the compounds of the present invention are formulated in a pharmaceutical formulation, which further includes vitamin E oil.

In another embodiment the compounds of the present invention are complexed with vitamin E oil in a pharmaceutical formulation.

In another embodiment the compounds of the present invention are formulated in a pharmaceutical formulation, which further includes a cyclodextrin.

In another embodiment the compounds of the present invention are formulated in a pharmaceutical formulation, which further includes Magnolol and/or its analogues and/or derivatives.

The present invention also provides a method for reducing the level of iron and/or copper in the cells of living subjects comprising administering one or more of the compounds of the present invention in a pharmaceutical formulation which further includes phosphatidyl-choline or di-phosphatidyl-choline.

The present invention also provides a method for treating degenerative and related disorders comprising administering one or more of the compounds of the present invention in a pharmaceutical formulation which further includes phosphatidyl-choline or di-phosphatidyl-choline.

The present invention also provides a method for treating degenerative and related disorders comprising administering one or more of the compounds of the present invention in a pharmaceutical formulation which further includes a cyclodextrin.

The present invention also provides a method for treating degenerative and related disorders comprising administering one or more of the compounds of the present invention in a pharmaceutical formulation which further includes magnolol and/or its analogues and/or derivatives.

The present invention also provides a method for treating degenerative and related disorders comprising administering a pharmaceutical formulation containing one or more D-amino acid oxidase inhibitors.

Preferably, the D-amino acids oxidase inhibitors are selected from one of the compounds of the present invention.

The present invention also provides a method for prophylactically and therapeutically treating degenerative and related disorders comprising administering to mammals a pharmaceutical formulation containing one or more inhibitors of the enzyme D-amino acid oxidase

In a preferred embodiment, the pharmaceutical formulation further contains glutathione precursors or regenerators.

In another embodiment, the glutathione precursors or regenerators are selected from the group comprising: N-acetylcysteine, 2-oxo-thiazolidine-4 carboxylic acid, timonacic acid, WR-2721 (Walter Reed), diethyidithocarbamate disulfiram (ANTABUSE), malotilate (Kantec), sulfarlem and oltipraz.

In another embodiment, the D-amino acids oxidase inhibitors are further selected from the group comprising: 2-oxo-3-pentynoate, acetylacetonate and kojic acid.

The present invention also provides a method for prophylactically and therapeutically treating cerebropathy comprising administering to a subject one or more of the compounds of the present invention.

The present invention also provides a method for prophylactically and therapeutically treating neurospanchnic disorders comprising administering to a subject one or more of the compounds of the present invention.

The present invention also provides an assay to determine oxidative stress to determine if a mammal has a degenerative or related disorder or the propensity of a mammal to develop such a disorder.

The present invention also provides an assay to determine oxidative stress to determine if a mammal has a degenerative or related disorder or the propensity of a mammal to develop such a disorder.

The oxidation of D-amino acids should be balanced by antioxidant mechanisms to keep ammonia and hydrogen peroxide levels in control. A blood assay is outlined which can determine the oxidative stress index of a mammal. Also, the oxidation of D-amino acids should be balanced by antioxidant mechanisms to keep ammonia and hydrogen peroxide levels in control. A blood assay is provided that can determine the oxidative stress index of a mammal.

The invention provides an assay to determine if a mammal has a degenerative or related disorder or the propensity to develop such a disorder, which comprises the following steps: taking a human circulatory fluid sample; splitting the human circulatory fluid into smaller samples; determining the hydrogen peroxide levels of one of the human circulatory fluid samples; treating another sample with D-amino acids; incubating; determining the hydrogen peroxide levels of the D-amino acid treated human circulatory fluid sample; and comparing the two samples.

A normal young mammal is be able to balance the generation of H₂O₂ and NH₄⁺ by the DAAO enzyme with generated glutathione for removal of the H₂O₂ together with catalase enzyme and the production of glutamate and neutralization glutamine for the removal of the NH4+. However in aged mammals and others (Alzheimer or Down Syndrome patients) with oxidative stress imbalances H₂O₂ and Ammonia will increase in concentration. A patient with potential Down syndrome, AD or probable AD will demonstrate increased hydrogen peroxide levels, prior to the onset of symptoms.

The present invention provides an assay to determine oxidative stress which will determine if a mammal has a degenerative or related disorder or the propensity of a mammal to develop such a disorder wherein the test determines the ammonia and/or hydrogen peroxide produced in a blood/serum sample of the mammal which is challenged with one or more D-amino acids.

The theory behind this assay is that a normal young mammal will be able to balance the generation of H₂O₂ and NH₄+. by the DAAO enzyme with generated glutathione for removal of the H₂O₂ together with catalase enzyme and the production of glutamate and neutralization glutamine for the removal of the NH4+. However in aged mammals and others (Alzheimer's patients) with oxidative stress imbalances H₂O₂ and Ammonia will increase in concentration and will slow this in the blood assay demonstrating their ability to contain their DAAO activity.

The invention provides for the use of the compunds disclosed herein, e.g., the dithiolthione compounds such as oltipraz, ADT or ADO, to inhibit the activity of the DAAO enzyme in vitro or in vivo.

According to the present invention, there is also provided a method of assaying for probable Alzheimer's disease in a human, which comprises determining in a sample of human circulatory fluid the amount of H₂O₂ present in the sample after said sample has been treated with a D-amino acid.

In another embodiment of the present invention method of assaying for probable Alzheimer's disease in a human is provided which comprises determining in a sample of human circulatory fluid the amount of ammonia present in the sample after said sample has been treated with a D-amino acid.

In a preferred embodiment the circulatory fluid is blood plasma and/or spinal fluid.

In a further embodiment of the present invention a method of assaying for probable Alzheimer's disease in a human is provided which comprises determining in a sample of human cerebrum material the amount of ammonia that is present in the sample after said sample has been treated with a D-amino acid.

An assay to determine if a mammal has a degenerative or related disorder or the propensity to develop such a disorder, which comprises the following steps: taking a sample of human circulatory fluid from a patient and a neurological control; determining the glutathione reductase levels of the human circulatory fluid samples; and comparing the levels of the two samples.

A patient with potential Down syndrome, Alzheimer's or probable Alzheimer's disease will demonstrate lower glutathione reductase levels, prior to the onset of symptoms, e.g., reduced by at least about 20% or by at least about 40% compared to normal controls.

The invention provides an assay to determine oxidative stress which will determine if a mammal has a degenerative or related disorder or the propensity of a mammal to develop such a disorder wherein the test determines the ammonia and/or hydrogen peroxide produced in a blood/serum sample of the mammal which is challenged with one or more D-amino acids.

In another embodiment the DAAO action is monitored by quantitative determination of the differing enzyme systems of the anti-oxidative system, e.g., glutathione reductase, in a study comparing a control group or individual to a group or individual that has or that is susceptible to a neurodegenerative disorder.

According to the present invention, there is also provided a method of assaying for probable Alzheimer's disease in a human, which comprises determining in a sample of human circulatory fluid the amount of H₂O₂ present in the sample after said sample has been treated with a D-amino acid.

In another embodiment of the present invention method of assaying for probable Alzheimer's disease in a human is provided which comprises determining in a sample of human circulatory fluid the amount of ammonia present in the sample after said sample has been treated with a D-amino acid.

In a preferred embodiment the circulatory fluid is blood plasma and/or spinal fluid.

In a further embodiment of the present invention a method of assaying for probable Alzheimer's disease in a human is provided which comprises determining in a sample of human cerebrum material the amount of ammonia present in the sample after said sample has been treated with a D-amino acid.

The invention provides a method to determine if a mammal (human) has a degenerative or related disorder or the propensity to develop such a disorder, which comprises the following steps: taking a circulatory fluid sample (e.g., blood serum or spinal fluid); removing the red blood cells from the serum; splitting the serum into smaller samples; determining the hydrogen peroxide levels of one of the serum samples; treating another sample with D-amino acids; incubating; determining the hydrogen peroxide levels of the D-amino acid treated serum sample; and comparing the two samples. In related embodiments, the assay determines oxidative stress or the DAAO action is monitored by PCR activity of the differing enzyme systems of the anti-oxidative system.

The invention provides a method of assaying for probable AD in a human, which comprises determining in a sample of human circulatory fluid the amount of H₂O₂ present in the sample after said sample has been treated with a D-amino acid. As used in any of the methods or assays disclosed or claimed herein, D-amino acids are typically selected from the D isomers of ala, phe, met, cys, tyr, val, leu, gly, arg, lys, glu, asp or ile.

A method of assaying for probable Alzheimer's disease in a human is provided which comprises determining in a sample of human circulatory fluid the amount of ammonia present in the sample after said sample has been treated with a D-amino acid. In a preferred embodiment the circulatory fluid is blood plasma and/or spinal fluid.

In a further embodiment the invention provides a method of assaying for probable Alzheimer's disease in a human is provided which comprises determining in a sample of human cerebrum material the amount of ammonia present in the sample after said sample has been treated with a suitable amount of one or more D-amino acids for a suitable time to detect ammonia.

An assay to determine oxidative stress which will determine if a mammal (human) has a degenerative or related disorder or the propensity of a mammal to develop such a disorder wherein the test determines the ammonia and/or hydrogen peroxide produced in a blood/serum sample of the mammal which is challenged with one or more D-amino acids. In another embodiment the DAAO action is monitored by PCR activity of the differing enzyme systems of the anti-oxidative system.

According to the present invention, there is also provided a method of assaying for probable Alzheimer's disease in a human, which comprises determining in a sample of human circulatory fluid the amount of H₂O₂ present in the sample after said sample has been treated with a D-amino acid.

In another embodiment of the present invention method of assaying for probable Alzheimer's disease in a human is provided which comprises determining in a sample of human circulatory fluid the amount of ammonia present in the sample after said sample has been treated with a D-amino acid. In a preferred embodiment the circulatory fluid is blood plasma and/or spinal fluid.

In a further embodiment of the present invention a method of assaying for probable Alzheimer's disease in a human is provided which comprises determining in a sample of human cerebrum material the amount of ammonia present in the sample after said sample has been treated with a D-amino acid.

Treatment Dosages and Formulations.

The treatment methods of the instant invention also comprise contacting a subject with (or administering to the subject or delivering to the subject's tissues) a therapeutically effective amount of a compound or mixture of compounds disclosed herein. Therapeutically effective amounts may be determined by methods well known to those skilled in the art, e.g., clinical trials, and daily doses may range up to about 4 grams per day, preferably about 50 milligrams to about 4 grams per day, e.g., 50 mg to 100 mg/day or about 150 mg/day (up to about 2.5 g/day). The instant compounds are preferably in the form of pharmaceutical compositions suitable for enteral, especially oral, administration to warm-blooded animals. The active ingredient in these compositions may be the instant compound, or a pharmaceutically acceptable salt, oxime, oxide, derivative, or metabolite thereof. These compositions may contain the active ingredient, i.e., a compound disclosed herein such as oltipraz, alone or, preferably in combination with a pharmaceutically acceptable excipient(s).

The compositions may be in dosage unit forms such as tablets, coated tablets, hard or soft gelatin capsules or syrups. These can be prepared using known procedures, for example by conventional mixing, granulating, tablet coating, dissolving or lyophilising processes. Thus, pharmaceutical compositions for oral administration can be obtained by combining the active ingredient with solid carriers, optionally granulating the resulting mixture, and processing the mixture or granulate, if desired or necessary after the addition of suitable excipients, to give tablets or coated tablet cores.

Suitable excipients are, in particular, fillers, such as sugars, for example lactose, sucrose, mannitol or sorbitol, cellulose preparations and/or calcium phosphates, for example tricalcium phosphate or calcium hydrogen phosphate, and binders, such as starches for example, corn, wheat, rice or potato starch, gelatin, tragacanth, methylcellulose and/or polyvinylpyrrolidone, and/or, if desired, disintegrants, such as the above mentioned starches, and also carboxymethyl starch, cross-linked polyvinylpyrrolidone, agar, alginic acid or a salt thereof such as sodium alginate, and/or flow regulators and lubricants, for example silica, talc, stearic acid or salts thereof such as magnesium stearate or calcium stearate, and/or polyethylene glycol. Coated tablet cores can be provided with suitable coatings, which if appropriate are resistant to gastric juices, using, inter-alia, concentrated sugar solutions which may contain gum arabic, talc, polyvinylpyrrolidone, polyethylene glycol and/or titanium dioxide, shellac solutions in suitable organic solvents or solvent mixtures or, for the preparation of coatings resistant to gastric Juices, solutions of suitable cellulose preparations such as acetylcellulose phthalate or hydroxypropylmethylcellulose phthalate. Dyes or pigments can be added to the tablets or coated tablets, for example to identify or indicate different doses of active ingredient.

The dosage, route of administration, and duration of therapy with the compounds of this invention, can readily be determined by those skilled in the art, which may be individualized according to the illness being treated, the patient's weight, the occurrences of another therapy employed in conjunction with the invention compounds and the patient's condition, clinical response and tolerance to the compounds.

These pharmaceutical compositions may be in dosage unit forms such as tablets, coated tablets, hard or soft gelatin capsules or syrups. These can be prepared using known procedures, for example by conventional mixing, granulating, tablet coating, dissolving or lyophilising processes. Thus, pharmaceutical compositions for oral administration can be obtained by combining the instant compound with a pharmaceutically acceptable carrier, preferably a solid carrier, optionally granulating the resulting mixture, and processing the mixture or granulate, if desired or necessary after the addition of suitable excipients, to give tablets or coated tablet cores.

Suitable excipients are, in particular, fillers, such as sugars, for example lactose, sucrose, mannitol or sorbitol, cellulose preparations and/or calcium phosphates, for example tricalcium phosphate or calcium hydrogen phosphate, and binders, such as starches for example, corn, wheat, rice or potato starch, gelatin, tragacanth, methylcellulose and/or polyvinylpyrrolidone, and/or, if desired, disintegrants, such as the above mentioned starches, and also carboxymethyl starch, cross-linked polyvinylpyrrolidone, agar, alginic acid or a salt thereof such as sodium alginate, and/or flow regulators and lubricants, for example silica, talc, stearic acid or salts thereof such as magnesium stearate or calcium stearate, and/or polyethylene glycol. Coated tablet cores can be provided with suitable coatings, which if appropriate are resistant to gastric juices, using, inter-alia, concentrated sugar solutions which may contain gum arabic, talc, polyvinylpyrrolidone, polyethylene glycol and/or titanium dioxide, shellac solutions in suitable organic solvents or solvent mixtures or, for the preparation of coatings resistant to gastric Juices, solutions of suitable cellulose preparations such as acetylcellulose phthalate or hydroxypropylmethylcellulose phthalate. Dyes or pigments can be added to the tablets or coated tablets, for example to identify or indicate different doses of active ingredient. The instant compounds, optionally in one or more of the various form described herein, may be contacted with the subject or administered enterally, parenterally (e.g., intravenously, intrathecally, intramuscularly or intraarterially), topically, orally, sublingually, by buccal administration, rectally, nasally, vaginally, transdermally or in any combination thereof.

The compounds disclosed herein are thus useful for the prevention or treatment of the symptoms of neurodegenerative disorders such as AD, or for treating or slowing progression of malaria or a trypanosome infection, or they are useful for enhancing the long term or short term memory in a subject in need thereof. The compounds are also useful for treating long term or short term memory loss associated with neurodegenerative disorders or related degenerative conditions, and for slowing the progression or rate of memory loss and for reducing the level of iron in the cells of a living subject, for inhibiting D-amino acid oxidase in a subject, and for enhancing a phase II detoxification enzyme in a subject, preferably selected from GST, γ-GCS, glutathione reductase, glutathione peroxidase, epoxide hydrase, AFB₁ aldehyde reductase, glucuronyl reductase, glucose-6-phosphate dehydrogenase, UDP-glucuronyl transferase and AND(P)H:quinone oxidoreductase.

The following are embodiments that further illustrate the invention and aspects thereof.

1. A method to treat or prevent a degenerative, neurodegenerative or related disorder comprising administering to a subject an effective amount of one or more compounds of the present invention.

2. The method of embodiment 1 wherein the compounds of the present invention are D-amino acid oxidase inhibitors.

3. The method of embodiment 1 wherein the compounds of the present invention enhance phase II detoxification enzymes.

4. The method of embodiment 3 wherein, the phase II detoxification enzymes that are enhanced by the compounds of the present invention are selected from the group consisting of GST, gamma-GST, glutathione reductase, glutathione peroxidase, epoxide hydrase, AFB₁ aldehyde reductase, glucuronyl reductase, glucose-6-phosphate dehydrogenase, UDP-glucuronyl transferase, and AND(P)H:quinone oxidoreductase.

5. The method of embodiment 1 wherein the compounds of the present invention have at least one adjunct residue, the at least one adjunct residue being attached to the compounds.

6. The method of embodiment 5 wherein the adjunct residue consists of one to eighty amino acids.

7. The method of embodiment 5 wherein the adjunct residue consists essentially of positive charged amino acids.

8. The method of embodiment 6 wherein the positive charged amino acid is Histidine, Arginine and/or Lysine.

9. The method of embodiment 5 wherein the adjunct residue consists of one to twenty amino acids of positive charge.

10. The method of embodiment 5 wherein the adjunct residue contains blocks of two or more adjacent amino acids of positive charge.

11. The method of embodiment 1 wherein the compounds of the present invention are metal chelating compounds.

12. The method of embodiment 11 wherein the metal chelating compounds specifically chelate iron and/or copper.

13. The method of embodiment 1 wherein the degenerative and related disorders are selected from the group comprising: Parkinson's disease, Huntington's disease, Amylotrophic Lateral Sclerosis, cognitive disorders, Progeria, Alzheimer's disease, epileptic dementia, presenile dementia, post traumatic dementia, senile dementia, vascular dementia and post stroke dementia, Down's syndrome, amyloidosis, amyloid associated with type II diabetes, Creutzfelt-Jakob disease, necrotic cell death, hypoxic damage, necrotic cell death, Gerstmann-Straussler syndrome, kuru and animal scrapie, amyloid associated with long-term hemodialysis and senile cardiac amyloid and Familial Amyloidotic Polyneuropathy, cerebropathy, neurospanchnic disorders, memory loss, aluminum intoxication, reperfusion injury, high iron levels in the cells of living subjects, high free transition metal ion levels in mammals, or toxic amounts of metal in the body or in certain body compartments.

14. The method of embodiment 1 wherein the degenerative disorders are neurodegenerative disorders optionally selected from Parkinson's disease, Alzheimer's disease, Huntington's disease, Amylotropic Lateral Sclerosis, epileptic dementia, presenile dementia, post traumatic dementia, senile dementia, vascular dementia and post stroke dementia, Down's syndrome, and Creutzfelt-Jakob disease.

15. The method of embodiment 1 wherein the compounds are formulated into a composition that further includes a pharmaceutically acceptable carrier.

16. The method of embodiment 1 wherein the compounds of the present invention are micronised.

17. The method of embodiment 1 wherein the compounds of the present invention are administered in an ophthalmic solution and are preferably in a pharmaceutical formulation that further includes an anti microbial preservative.

18. The method of embodiment 1 wherein the compounds of the present invention are formulated in a pharmaceutical formulation, which further includes phosphatidylcholine or diphosphatidylcholine.

19. The method of embodiment 1 wherein the compounds of the present invention are complexed with phosphatidyl-choline or di-phosphatidyl-choline in a pharmaceutical formulation.

20. The method of embodiment 1 wherein the compounds of the present invention are formulated in a pharmaceutical formulation, which further includes vitamin E oil.

21. The method of embodiment 1 wherein the compounds of the present invention are completed with vitamin E oil in a pharmaceutical formulation.

22. The method of embodiment 1 wherein the compounds of the present invention are formulated in a pharmaceutical formulation, which further includes a cyclodextrin.

23. The method of embodiment 1 wherein the compounds of the present invention are formulated in a pharmaceutical formulation, which further includes Magnolol and/or its analogues and/or derivatives.

24. The method of embodiment 1 wherein the compounds of the present invention are formulated in a pharmaceutical formulation further contains glutathione precursors or regenerators.

25. The method of embodiment 24 wherein the glutathione precursors or regenerators are selected from the group comprising: N-acetylcystein, 2-oxo-thiazolidine-4 carboxylic acid, timonacic acid and WR-2721 (Walter Reed), diethyldithocarbamate disulfiram (ANTABUSE) Malotilate (Kantec), Sulfarlem and Oltipraz.

26. The method of embodiment 1 wherein said subject is a neonate and said administering is effected prior to delivery of said neonate and/or during delivery of said neonate.

27. The method of embodiment 1 wherein the compounds are administered enterally, parenterally, topically, orally, rectally, nasally or vaginally.

28. The method of embodiment 1 wherein the compounds are administered intermittently.

29. The method of embodiment 1 wherein the compounds are selected from the group comprising compounds of the formula given for FIG. 1, FIG. 2, FIG. 3 or FIG. 4, and their oximes, oxides, derivatives or metabolites.

30. The method of embodiment 19 wherein the compounds of the present invention are oltipraz, ADT, ADO, malotilate, 1,2-dithiole-3-thione, 1,3-dithiole-2-thione, lipoamide or [1,2]dithiolo[4,3-c]-1,2dithiole-3,6-dithione.

31. The method of embodiment 19 wherein the derivatives of the compounds of the present invention are compounds having the formulas given for FIG. 8, FIG. 9, FIG. 10, FIG. 11, FIG. 50 or FIG. 51.

32. The method of embodiment 31 wherein R₁ and R₂ together form a mono- or polycyclic C₂-C₂₀ alkylene group optionally comprising one or more hetero atoms, with the exception of the 2,2 dimethyltrimethylene group, or a C₃-C₁₂ cycloalkylene group, and R is chosen from a C₁-C₆, alkyl group, and their pharmaceutically acceptable salts,

33. The method of embodiment 31 wherein another group of compounds is formed when A (FIGS. 10 & 11) is a group C═N—OR′₃ where R′₃ is an optionally substituted C₁-C₆ alkyl group, in particular substituted with one or more groups chosen from hydroxyl, amino, chloro, bromo, fluro, iodo and C₁-C₄ alkoxy groups, or an aryl (C₁-C₆ alkyl) group, i.e., compounds of the formula given for FIG. 16 or FIG. 18.

34. The method of embodiment 31 wherein another group of compounds is formed when A (FIGS. 10 & 11) is a group C═N—O—CO—R″₃, R″₃ being chosen from a hydrogen atom, an optionally substituted C₁-C₆ alkyl group, an aryl group and an aryl (C₁-C₆ alkyl) group, that is to say compounds of formula given for FIG. 19 wherein R″₃ is —H, optionally substituted C₁-C₆ alkyl or aryl.

35. The method of embodiment 31 wherein another group of compounds is formed when A (FIGS. 10 & 11) is a CH—OH group, i.e., the compounds of formula given for FIG. 20.

36. The method of embodiment 31 wherein another group of compounds is formed when A (FIGS. 10 & 11) is a group C═N—R, R, being a C₁-C₆ alkyl or aryl, i.e., the FIG. 21 compounds.

37. The method of embodiment 31 wherein another group of compounds is formed when A (FIGS. 10 & 11) is a C═O group and R²⁴ is an oxygen atom, i.e., the FIG. 22 compounds.

38. The method of embodiment 37 wherein R₁ and R₂ together form a mono- or polycyclic C₂-C₂₀ alkylene group optionally comprising one or more hetero atoms.

39. The method of embodiment 37 wherein another group of compounds is formed in which R₂ is chosen from C₁-C₆ alkyl C₂-C₆ alkenyl. aryl. aryl(C₁-C₆ alkyl). aryl C₂-C₆ alkenyl. terpenyl. C₂-C₆ alkynl. C₂-C₆ alkynyl substituted with C₁-C₆ alkyl or aryl.

40. The method of embodiment 31 wherein another group of compounds is formed in which R is chosen from C₁-C₆ alkyl.

41. The method of embodiment 31 wherein oximes of derivatives of the present invention are compounds of FIG. 12, FIG. 13 or FIG. 14.

42. The method of embodiments 29-41 wherein the compounds are FIG. 15 compounds.

43. The method of embodiments 29-42 wherein the compounds are FIG. 28 compounds.

44. The method of embodiment 43 wherein the compounds of the formula shown in FIG. 28, can be selected from the group comprising:

5-(4-phenyl-1,3-butadienyl)-1,2-dithol-3-thione;

5-4(4-chlorophenyl)-1,3-butadienyl-1,2-dithiol-3-thione;

5-{4-(4-methoxyphenyl)-1,3-butadienyl}-1,2-dithiol-3-thione;

5-{4-(p-toluyl)-1,3-butadienyl}-1,2-dithol-3-thione;

5-{4-(o-chlorophenyl)-1,3-butadienyl}-1,2-dithi-ol-3-thione; and

5-{4-(m-methylphenyl)-1,3butadienyl}-1,2-ffithiol-3-thione.

45. The method of embodiment 29 wherein the 1,2-dithiole is a FIG. 34 compound.

46. The method of embodiment 45, wherein Het is pyrimidin-2-yl, pyrimidin-4-yl, or pyrimidin-5-yl, or pyrimidin-2-yl, pyrimidin-4-yl, or pyrimidin-5-yl substituted by halogen, alkyl of 1 through 4 carbon atoms, alkoxy of 1 through 4 carbon atoms, alkylthio of 1 through 4 carbon atoms, or dialkylamino having 1 through 4 carbon atoms in each alky], and R represents alkyl of 1 through 4 carbon atoms, carboxy, alkoxycarbonyl having 1 through 4 carbon atoms in the alkoxy, carbamoyl, or N-alkylcarbamoyl having 1 through 4 carbon atoms in the alkyl.

47. The method of embodiment 45, wherein Het is pyrimidin-2-yl, pyrimidin-4-yl, or pydmidin-5-yl, or pyrimidin-2-yl, pyrimidin-4-yl or pyrimidin-5-yl substituted by halogen, alkyl of 1 through 4 carbon atoms, alkylthio of 1 through 4 carbon atoms, or dialkylamino having 1 through 4 carbon atoms in each alky], and R represents alkyl of 1 through 4 carbon atoms, alkoxycarbonyl having 1 through 4 carbon atoms in the alkoxy, or R,—CH(OH)— in which Rl represents hydrogen or alkyl of 1 through 3 carbon atoms.

48. The method of embodiments 45 to 47, wherein the compounds of are 4-ethyl-5-(pyrimidin-4-yl)-1,2-dithiole-3-thione, 4-methyl-5-(5-methylthiopyrimidin-4-yl)-1,2-dithiole-3-thione or 5-(5-chloropyrimidin-4-yl)-4-methyl-1,2-dithiole-3-thione.

49. The method of embodiment 29 wherein the 1,2-dithiol-3-thiole-S-oxides have the following formula given for FIG. 35.

50. The method of embodiment 49, wherein another group of compounds is formed in which R1 is selected from the group consisting of fluorine, chlorine, bromine, iodine and methoxy, and R₂ is hydrogen.

51. The method of embodiment 29, wherein the 1,3-dithiolo(4.5-d)-1,3-(dithiino-2-thione) compound is a FIG. 5 compound, including 1,3-dithiolo(4.5-d)-1,3-dithiole-2-thione, 5-chloro-1,3-dithiolo (4.5-d)-1,3-dithiole-2-thione or 5-cyano-1,3-dithiolo(4.5-d)-1,3-dithiole-2-thione.

52. The method of embodiment 31, wherein the 1,3-dithiole derivatives are compounds of FIG. 6.

53. The method of embodiment 42, wherein another group of compounds is formed in which R¹ and R² together form —(CH₂)₄—, —(CH₂)₅—, ——(CH₂)₆—, —CH₂OCH₂CH₂—, —CH₂SCH₂CH₂—, —CH₂CH₂SCH₂CH₂CH₂—, —CH₂CH₂OCH₂CH₂—, —CH₂CH₂NHCH₂CH₂—, —CH₂CH₂N(ph)₂CH₂CH₂—, ——CH₂CH₂N(CH₂ph)CH₂CH₂—, —CH₂CH₂(CH₃) CH₂CH₂—, —CH₂CH═CHC—H₂—CH₂CH═CHCH₂CH₂—, which may be substituted by carboxyl, methyl, ethyl, n-propyl, isopropyl, n-butyl, n-pentyl, n-hexyl, methoxycarbonyl, ethoxycarbonyl, isopropoxycarbonyl, carbamoyl, N-methylcarbamoyl, N,N-dimethylcarbamoyl, N-phenylcarbamoyl or N-benzylcarbamoyl, and Q is an acid residue of hydrochloric acid, hydrobromic acid, hydroiodic acid, nitric acid, perchlonc acid, borofluoric acid, sulfuric acid, phosphoric acid, oxalic acid, tartaric acid, citric acid, methanesulfonic acid or p-toluenesulfonic acid.

54. The method of embodiment 42, wherein another group of compounds is formed in which the moiety is:

2-ethoxycarbonylpyrrolidinium, 2-carboxypyrrolidinium, 2-carbamoylpyrrolidinium,

4-ethoxycarbonylthiazolidinium, 2-ethoxycarbonylpiperidinium,

3-ethoxycarbonylpiperidirtium, 4-ethoxycarbonylpiperidinium, 4-carboxypiperidinium,

4-carbamoylpijperidinium, 3-ethoxycarbonyl-6-methyl piperidinium or

4-ethoxycarbonylpiperazinium, and Q is C10₄, Cl, Br, 1 or HSO₄.

55. The method of embodiment 29, wherein the 1,2-dithiol-3-ylideneammonium derivatives have the general formula given for FIG. 7 compounds.

56. The method of embodiment 55, wherein 1,2-dithio-1-3-ylideneammonium derivatives are selected from

N-[5-(4-chlorophenacylthio)-1 2-dithiol-3-ylidene] morpholinflum chloride;

N-[5-(3-methoxyphenacylthio)-1,2-dithiol-3-ylidene]-morpholinium chloride;

N-[5-(4-fluorophenacylthio)-1,2-dithiol-3-ylidene]-morpholinium chloride;

N-[5-(2,4-dichlorophenacylthio)-1,2-dithiol-3-ylidene]-morpholinium chloride;

N-[5-(2-chlorophenacylthio)-1,2-ditliiol-3-ylidene]-morpholinium iodide;

N-[5-(4-hydroxyphenacylthio)-1,2-dithiol-3-ylidene]-morpholinium chloride;

N-[5-(4-methoxyphenacylthio)-1,2-dithiol-3-yllidene]-morpholinium iodide;

N-[5-(4-methylphenacylthio)-1,2-dithiol-.3-ylidene]-morpholinium chloride;

N-[5-(4-cyanophenacylthio)-1,2-dithiol-3-ylidene]-morpholinium chloride; and

N-[5-(phenacylthio)-1,2dithiol-3-ylideene]-morpholinium chloride.

60. The method of embodiment 1, wherein the compounds of the present invention are isobenzothiazolone derivatives given for FIG. 24.

61. The method of embodiment 60, wherein R¹ is nitro or arylazo and R² as hydrogen.

62. The method of embodiment 60, wherein R² is hydrogen and R¹ is phenylazo; substituted arylazo such as 4-hydroxyphenylazo; 4-nitro-2-methylphenylazo; 2-hydroxy-l-napthylazo; 2-hydroxy-5-methylphenylazo; 2-hydroxy4-methyl-5-nitrophenylazo; 4-hydroxy-l-napthylazo; 4-hydroxy-3-methyl-1-napthylazo; 4-hydroxy-5-aza-1-napthylazo; 2-amino-l-napthylazo; 1-hydroxy-2-napthylazo; 3-N,N-dimethylaminopropylcarboxyamido-1-hydroxy-4-naphthylazo; 1-hydroxy-4-methoxy-2-naphthylazo, 2-hydroxy-3-carboxy-l-naphthylazo; 1-hydroxy-3,6-disulfonato-2-naphthylazo; 2,3-dihydroxy-1-naphthylazo; or 2-hydroxy-3,5-dimethyl-1-phenylazo. In one particular embodiment R¹ is the substituted benzylideneamino, 2,4-dinitrobenzylideneamino and R² is hydrogen.

63. The method of embodiment 60, wherein R³ may be —(CH₂)nR⁴R⁵ where n is from 2 to 6, 3-carboxypropyl, sulfonatoethyl and polyethyl ethers of the type —CH₂(C—H₂OCH₂),CH₃ where n is less than 10 and R⁴ and R⁵ are simple alkyls or hydrogens.

64. The method of embodiment, wherein R³ side chains are aminoalkyl, carboxyalkyl, omega amino polyethyl ethers, N-haloacetyl derivatives, alkyl, aryl, heteroaryl, alkoxy, hydroxy, amino groups, aminoalkyl, carboxyalkyl, hydroxyalkyl or haloalkyl, aminoaryl, carboxyryl or hydroxyaryl groups.

65. The method of embodiment 1, wherein the compounds of the present invention are isobenzothiazolone derivatives with the FIG. 25 structure wherein at least one of R¹ and R² is nitro, arylazo, substituted arylazo, benzylideneamino or substituted benzyfideneamino and one of R¹ and R² may be hydrogen and R³ is a aminoallayl, aminoaryl and aminoheteroaryl, carboxyalkyl, carboxyaryl or carboxyheteroaryl covalently linked to a polymer comprising amino or hydroxy groups. The spacer arm R³ can comprise oligmers or polyethylene-glycol and its derivatives. In one aspect, R³ may be 17-chloracetamido-3,6,9,12, 1 5-pentaoxyhep-tadecyl where hexaethylene glycol has been chloroacetamidated. When the polymer groups, Y¹ and R³ comprises carboxyl groups, the covalent linkage is preferably through an ester bond. When the polymer comprises amino groups, the analog covalent linkage is through an amide bond. The amine bearing polymer, when coupled to R³, may be a polymer such as chitosan, polyalkylamine, aminodextran, polyethyleneimine, polylysine or amityrene.

66. The method of embodiment 65, wherein the R³ substituents comprise an alkyl linked to an amine bearing polymer by amine displacement of a halogen from an alpha-haloalkyl or alpha-haloalkylcarbox amido R³ precursor. In the case of aminoalkyl or aminoaryl groups the R³ substituent may also be covalently linked to a polymer such as polyepichlorohydrin, chloromethyl-polystyrene, polyvinylalcohol or polyvinylpyridine. The R³ substituent of the present invention may generally be an aminoalkyl, hydroxyalkyl, aminoaryl or hydroxyary] group linked to a polymer comprising carboxyl groups through amide or ester linkages.

67. The method of embodiment 65, wherein when polymers are involved in the R³ structure, the polymer may be one such as polyacrylic acid, polymethacrylic acid, polyitaconic acid, oxidized polyethylene oxide, poly (methylmethacrylate/methacrylic acid), carboxyinethyl cellulose, carboxymethyl agarose or carboxymethyl dextran. When such a carboxyl polymer is involved, the R³ may be aminoalkyl (such as 8 aminohexyl, for example), hydroxyalkyl, aminoaryl or hydroxyaryl linked to the polymer through amide or ester linkages. In such cases, an R³ precursor function may bear an amine or hydroxyl group to be covalently linked to a polymer by reaction with an acid anhydride-bearing polymer or by coupling with a carboxylate bearing polymer through carbodimide induced bond formation.

68. The method of embodiment 65, wherein the R³ substituent or precursor thereto in the compound of the present invention may also be a haloalkyl or carboxylialoalkyl moiety such as chloracetamido. Such a substituent may readily coupled to an amine bearing polymer by amine displacement of the halogen.

69. The method of embodiment 1, wherein the compounds are further selected from the FIG. 23 compounds.

70. The method of embodiment 69, wherein R₁ and R₂ together form a mono- or polycyclic C₂-C₂₀ alkylene group optionally comprising one or more heteroatoms (e.g., O, N or S).

71. The method of embodiment 69 wherein, R is chosen from a C₁-C₆, alkyl group, and their pharmaceutically acceptable salts.

72. The method of embodiment 69, wherein the aryl group or aryl fraction of an arylalkyl group denotes an aromatic carbon-based group such as a phenyl or naphthyl group or an aromatic heterocyclic group such as a thienyl of furyl group, it being possible for these groups to bear one or more substituents chosen from a halogen atom, a C₁-C₄ alkyl group, a C₁-C₄ alkoxy group, a trifluoromethyl group, a nitro group and a hydroxyl group.

73. The method of embodiment 1, wherein the compounds are further selected from FIG. 26 or FIG. 27 compounds or compounds.

74. The method of embodiment 73, wherein the compound is selected from the group comprising: 3-keto lipoic acid, 3-hydroxy lipoic acid, 3-keto dihydrolipoic acid or 3-hydroxy dihydrolipoic acid.

75. The method of embodiment 1, wherein the compounds are further selected from the group comprising compounds of FIG. 29, FIG. 30, FIG. 31, FIG. 31a, FIG. 32 and FIG. 33.

76. The method of embodiment 1, wherein the compounds are further selected compounds given for FIG. 36 and FIG. 37.

77. The method of embodiment 76, wherein another group of compounds is formed in which Y is nitro and n is 1.

78. The method of embodiment 76, wherein another group of compounds is formed in which Y is trifluoromethyl and n is 1.

79. The method of embodiment 76, wherein another group of compounds is formed in which Y is trifluoromethyl and n is 2.

80. The method of embodiment 76, wherein another group of compounds is formed in which Y is nitro and n is 2.

81. The method of embodiment 76, wherein another group of compounds is formed in which Y is CF₃ and n is 2.

82. The method of embodiment 76, wherein another group of compounds is formed in which Y is CF₃ and n is 2.

83. The method of embodiments 76 to 82, wherein the compound is:

S-tertbutyl-S′-(2,4-dinitro-3-aminopropyl-6-tri-fluoromethylphenyl)-trithiocarbonate

84. The method of embodiment 1 wherein the compound has the formula given for FIG. 38 compounds.

85. The method of embodiment 1 wherein the compound has the formula given for FIG. 39 compounds.

86. The method of embodiment 85, wherein another group of compounds is formed in which R₁ and R₂ are independently selected from the group consisting of hydrogen, C₁₋₄ alkoxy groups, and C₂₋₄ alkenyl groups.

87. The method of embodiment 85, wherein another group of compounds is formed in which R₁ and R₂ are each hydrogen.

88. The method of embodiment 1 wherein the compound has the formula given for FIG. 40 compounds.

89. The method of embodiment 88, wherein another group of compounds is formed in which R and R1 are branched-chain alkyl radicals having from 3 to 8 carbon atoms, 1-methyl cyclohexyl or aa-dimethyl benzyl.

90. The method of embodiment 88, wherein another group of compounds is formed in which Y is an —S-alkyl group having from 6 to 18 carbon atoms.

91. The method of embodiments 88 to 90, wherein the compounds are selected from the group comprising:

4-(3,5-di-isopropyl-4-hydroxyphenyl)-1,2-dithiole-3-thione;

4-(3,5-di-t-butyl-4-hydroxyphenyl)-1,2-dithiole-3-thione;

4-[3,5-bis(l,l-dimethylpropyl)-4-hydroxyphenyl]-1,2-dithi-ole-3-thione;

4-[3,5bis(l,l-dimethylbutyl)-4-hydroxyphenyl]-1,2-dithiole-3-thione;

4-[3,5-bis(1,1,3,3-tetramethylbutyl)-4-hydroxyphenyl]-1,2-dithole-3-thione;

4-[3,5-bis(l-methylcyclohexyl)-4-hydroxyphenyl]-1,2-dithiole-3-thione;

4-[3,5-bis(l,l-dimethylbenzyl)-4-hydroxyphenyl]-1,2-dithiole-3-thione;

4-(3t-butyl-4-hydroxy-S-isopropylphenyl)-1,2-dithiole-3-thione;

4-(3t-butyl-4-hydroxy-5-methylphenyl)-1,2-dithiole-3-thione;

4-[3(1,1-dimethylpropyl)-4-hydroxy.-5-isopropylphenyl]-1,2-dithiole-3-thione;

4-[3(1,1-dimethylbenzyl)-4-hydroxy-5-isopropylphenyl]-1,2-dithiole-3-thione;

5-benzylthio-4-(3,5-di-t-butyl-4-hydroxyphenyl)-1,2-dithiole-3-thione;

5-benzylthio-4-[3,5-bis(l,l-dimethylpropyl)-4-hydroxy-phenyl]-1,2-dithiole-3-thione;

5-hexylthio-4-(3,5-di-t-butyl-4-hydroxyphenyl)-1,2-dithiole-3-thione;

5-hexylthio-4-[3,5-bis(l,l-dimethylbutyl)-4-hydroxy-phenyl]-1,2-dithiole-3-thione;

5-octadecylthio-4-(3,5-di-t-butyl-4-hydroxyphenyl)-1,2-dithiole-3-thione;

5-octadecylthio-4-[3,5-bis(l,l-dimethylbenzyl)-4-hydroxyphenyl]-1,2-dithiole-3-thione;

5-allylthio-4-(3,5-di-t-butyl-4-hydroxyphenyl)-1,2-dithiole-3-thione;

5-cyclohexylthio-4-(3,5-di-t-butyl-4-hydroxyphenyl)-1,2-dithiole-3-thione; and

4-(3,5-di-sec-butyl-4-hydroxyphenyl)-1,2-dithiole-3-thione.

92. The method of embodiment 88, wherein another group of compounds is formed in which Y is the residue of a FIG. 41 compound.

93. The method of embodiment 1, wherein the compounds of the present invention have the formula given for FIG. 42 compounds

94. The method of embodiment 93, wherein another group of compounds is formed in which A is a methylene group and R2 is a hydrogen atom or a salt thereof.

95. The method of embodiment 93, wherein another group of compounds is formed in which R′ is a hydrogen atom, a hydroxyl group or a lower alkoxy group; or a salt thereof.

96. The method of embodiment 93, wherein another group of compounds is formed in which A is an oxygen atom and R2 is a hydrogen atom; or a salt thereof or alternatively R, is a hydrogen atom, a hydroxyl group or a lower alkoxy group, or a salt thereof.

97. The method of embodiments 93 to 96, wherein the compound is a compound of structure (a) to (k) or a salt thereof.

98. The method of embodiments 93 to 97, wherein the compounds of the present invention have the formula given for FIG. 43 compounds.

99. The method of embodiment 98, wherein another group of compounds is formed in which R¹¹ is an alkyl group; or a salt thereof.

100. The method of embodiment 98, wherein another group of compounds is formed in which R¹¹ is —(CH₂)ₘ—C₆H₂—R12R13R14, wherein m, R12, R13 and R14 are the same as defined above and the sulfo group bonds to the 3-position, X is a 4-methoxy group, and R12, R13, R14 and Y are each a hydrogen atom); or a salt thereof.

101. The method of embodiments 98 to 100 wherein the following compounds; or a salt thereof can be selected from the group comprising:

5-Hexyl-4-(4-methoxy-3-suifobenzyl)-3H-1,2-dithiole-3-thione; and

4-(4-Methoxy-3-suifophenyi)-5-(p-toiyi)-3H-1,2-dithiole-3-thione.

102. The method of embodiment 2 wherein, the D-amino acids oxidase inhibitors are further selected from the group comprising: 2-oxo-3-pentynoate; acetylacetonate and kojic acid.

103. A method to treat or prevent malaria or a trypanosome infection comprising administering to a subject an effective amount of one or more compounds of the present invention.

104. The method of embodiment 103 wherein, the compounds of the present invention are D-amino acid oxidase inhibitors.

105. The method as claimed in claim 103 wherein, the compounds of the present invention enhance phase II detoxification enzymes.

106. The method of embodiment 105 wherein, the phase II detoxification enzymes that are enhanced by the compounds of the present invention are selected from the group comprising: GST, gamma-GST, glutathione reductase, glutathione peroxidase, epoxide hydrase, AFB₁ aldehyde reductase, glucuronyl reductase, glucose-6-phosphate dehydrogenase, UDP-glucuronyl transferase, and AND(P)H:quinone oxidoreductase.

107. The method of embodiment 103 wherein, the compounds of the present invention have at least one adjunct residue, the at least one adjunct residue being attached to the compounds.

108. The method of embodiment 107 wherein, the adjunct residue consists of one to eighty amino acids.

109. The method of embodiment 107 wherein, the adjunct residue consists essentially of positive charged amino acids.

110. The method of embodiment 108 wherein, the positive charged amino acids independently are histidine, arginine or lysine.

111. The method of embodiment 107 wherein, the adjunct residue consists of one to twenty amino acids of positive charge.

112. The method of embodiment 107 wherein, the adjunct residue contains blocks of two or more adjacent amino acids of positive charge.

113. The method of embodiment 103 wherein, the compounds of the present invention are metal chelating compounds.

114. The method of embodiment 113 wherein, the metal chelating compounds specifically chelate iron and/or copper.

115. The method of embodiment 103 wherein, the compounds are formulated into a composition that further includes a pharmaceutically acceptable carrier.

116. The method of embodiment 103 wherein, the compounds of the present invention are micronised.

117. The method of embodiment 103 wherein, the compounds of the present invention are administered in an ophthalmic solution and are preferably in a pharmaceutical formulation that further includes an anti microbial preservative.

118. The method of embodiment 103 wherein, the compounds of the present invention are formulated in a pharmaceutical formulation, which further includes phosphatidyl-choline or di-phosphatidyl-choline.

119. The method of embodiment 103 wherein, the compounds of the present invention are complexed with phosphatidyl-choline or di-phosphatidyl-choline in a pharmaceutical formulation.

120. The method of embodiment 103 wherein, the compounds of the present invention are formulated in a pharmaceutical formulation, which further includes vitamin E oil.

121. The method of embodiment 103 wherein, the compounds of the present invention are complexed with vitamin E oil in a pharmaceutical formulation.

122. The method of embodiment 103 wherein, the compounds of the present invention are formulated in a pharmaceutical formulation, which further includes a cyclodextrin.

123. The method of embodiment 103 wherein, the compounds of the present invention are formulated in a pharmaceutical formulation, which further includes Magnolol and/or its analogues and/or derivatives.

124. The method of embodiment 103 wherein, the compounds of the present invention are formulated in a pharmaceutical formulation, further contains glutathione precursors or regenerators.

125. The method of embodiment 124, the glutathione precursors or regenerators are selected from the group comprising: N-acetylcystein, 2-oxo-thiazolidine-4 carboxylic acid, timonacic acid and WR-2721 (Walter Reed), diethyldithocarbamate disulfiram (ANTABUSE) malotilate (Kantec), sulfarlem and oltipraz.

126. The method of embodiment 103 wherein, said subject is a neonate and said administering is effected prior to delivery of said neonate and/or during delivery of said neonate.

127. The method of embodiment 103 wherein, the compounds are administered enterally, parenterally, topically, orally, rectally, nasally or vaginally.

128. The method of embodiment 103 wherein, the compounds are administered intermittently.

129. The method of embodiment 103 wherein, the compounds are selected from the group comprising: 1,2-Dithiolane Class 1,1,2-Dithiole Class 2 1,3-Dithiole Class 3 and 1,3-Dithiolane Class 4 and their oximes, oxides, derivatives or metabolites.

130. The method of embodiment 129 wherein, the compounds of the present invention are selected from oltipraz, ADT, ADO, 1,3-dithiole-2-thione, lipoamide (1,2-dithiolane), [1,2]dithiolo[4,3-c]-1,2-dithiole-3,6-dithione, malotilate and 1,2-dithiole-3-thione.

131. The method of embodiment 129 wherein, the derivatives of the compounds of the present invention include compounds of FIG. 8, FIG. 9, FIG. 10 and FIG. 11.

132. The method of embodiment 131, wherein R₁ and R₂ together form a mono- or polycyclic C₂-C₂₀ alkylene group optionally comprising one or more hetero atoms, with the exception of the 2,2 dimethyltrimethylene group, or a C₃-C₁₂ cycloalkylene group, and

R is chosen from a C₁-C₆, alkyl group, and their pharmaceutically acceptable salts.

133. The method of embodiment 131, wherein another group of compounds is formed when A (FIGS. 10 & 11) is a group C═N—OR′₃ where R′₃ is an optionally substituted C₁-C₆ alkyl group, in particular substituted with one or more groups chosen from hydroxyl, amino, chloro, bromo, fluro, iodo and C₁-C₄ alkoxy groups, or an aryl (C₁-C₆ alkyl) group, i.e., FIG. 16 or FIG. 18 compounds.

134. The method of embodiment 131, wherein another group of compounds is formed when A (FIGS. 10 & 11) is a group C═N—O—CO—R″₃, R″₃ being chosen from a hydrogen atom, an optionally substituted C₁-C₆ alkyl group, an aryl group and an aryl (C₁-C₆ alkyl) group, i.e., FIG. 19 compounds.

135. The method of embodiment 131, wherein another group of compounds is formed when A (FIGS. 10 & 11) is a CH—OH group, i.e., FIG. 20 compounds.

136. The method of embodiment 131, wherein another group of compounds is formed when A (FIGS. 10 & 11) is a group C═N—R, R, being a C₁-C₆ alkyl or an aryl group, i.e., FIG. 21 compounds.

137. The method of embodiment 131, wherein another group of compounds is formed when A (FIGS. 10 & 11) is a C═O group and R²⁴ is an oxygen atom, i.e., FIG. 22 compounds.

138. The method of embodiment 137, wherein R₁ and R₂ together form a mono- or polycyclic C₂-C₂₀ alkylene group optionally comprising one or more independently selected heteroatoms (e.g., O, N or S).

139. The method of embodiment 137, wherein another group of compounds is formed in which R₂ is chosen from C₁-C₆ alkyl C₂-C₆ alkenyl. aryl. aryl(C₁-C₆ alkyl). aryl C₂-C₆ alkenyl. terpenyl. C₂-C₆ alkynl. C₂-C₆ alkynyl substituted with C₁-C₆ alkyl or aryl.

140. The method of embodiment 131, wherein another group of compounds is formed in which R is chosen from C₁-C₆ alkyl.

141. The method of embodiment 131, wherein oximes of derivatives of the present invention include compounds of FIG. 12, FIG. 13 or FIG. 14.

142. The method of embodiments 129 to 141 wherein the compounds are incorporated such as shown in FIG. 15.

143. The method of embodiments 139 to 142 wherein the compounds have a formula shown in FIG. 28.

144. The method of embodiment 143, wherein the compounds of the formula shown in FIG. 28, can be selected from the group comprising:

5-(4-phenyl-1,3-butadienyl)-1,2-dithiol-3-thione;

5-4(4-chlorophenyl)-1,3-butadienyl-1,2-dithiol-3-thione;

5-{4-(4-methoxyphenyl)-1,3-butadienyl}-1,2-dithiol-3-thione;

5-{4-(p-toluyl)-1,3-butadienyl}-1,2-dithiol-3-thione;

5-{4-(o-chlorophenyl)-1,3-butadienyl}-1,2-dithi-ol-3-thione; and

5-{4-(m-methylphenyl)-1,3butadienyl}-1,2-ffithiol-3-thione.

145. The method of embodiment 129 wherein the of a group of 1,2-dithiole can have the formula of FIG. 34.

148. The method of embodiment 145, wherein the compounds of the formula, can be selected from the group comprising:

4-ethyl-5-(pyrimidin-S-yl)-1,2-dithole-3-thione;

4-methyl-5-(5-methylthiopyrimidin-4-yl)-1,2-dithole-3-thione.

5-(Schloropyrimidiri-4yl)-4-methyl-1,2-dithiole-3-thione.

149. The method of embodiment 129 wherein the 1,2-dithiol-3-thion-S-oxides have the formula of FIG. 35.

150. The method of embodiment 149, wherein another group of compounds is formed in which R1 is selected from the group consisting of fluorine, chlorine, Bromine, iodine and methoxy, and R₂ is hydrogen.

151. The method of embodiment 129, wherein the 1,3-dithiolo(4.5-d)-1,3-(dithiino-2-thiole) compounds are ones having the FIG. 5 formula.

152. The method of embodiment 131, wherein the 1,3-dithiole derivatives have the FIG. 6 formula.

153. The method of embodiment 152, wherein another group of compounds is formed in which R¹ and R² together form —(CH₂)₄—, —(CH₂)₅—, ——(CH₂)₆—, —CH₂OCH₂CH₂—, —CH₂SCH₂CH₂—, —CH₂CH₂SCH₂CH₂CH₂—, —CH₂CH₂OCH₂CH₂—, —CH₂CH₂NHCH₂CH₂—, —CH₂CH₂N(ph)₂CH₂CH₂—, ——CH₂CH₂N(CH₂ph)CH₂CH₂—, —CH₂CH₂(CH₃) CH₂CH₂—, —CH₂CH═CHC—H₂—CH₂CH═CHCH₂CH₂—, which may be substituted by carboxyl, methyl, ethyl, n-propyl, isopropyl, n-butyl, n-pentyl, n-hexyl, methoxycarbonyl, ethoxycarbonyl, isopropoxycarbonyl, carbamoyl, N-methylcarbamoyl, N,N-dimethylcarbamoyl, N-phenylcarbamoyl or N-benzylcarbamoyl, and Q is an acid residue of hydrochloric acid, hydrobromic acid, hydroiodic acid, nitric acid, perchlonc acid, borofluoric acid, sulfuric acid, phosphoric acid, oxalic acid, tartaric acid, citric acid, methanesulfonic acid or p-toluenesulfonic acid.

154. The method of embodiment 152, wherein another group of compounds is formed in which the moiety is:

2-ethoxycarbonylpyrrolidinium, 2-carboxypyrrolidinium, 2-carbamoylpyrrolidinium,

4-ethoxycarbonylthiazolidinium, 2-ethoxycarbonylpiperidinium,

3-ethoxycarbonylpiperidirtium, 4-ethoxycarbonylpiperidinium, 4-carboxypiperidinium,

4-carbamoylpijperidinium, 3-ethoxycarbonyl-6-methyl piperidinium or

4-ethoxycarbonylpiperazinium, and Q is C10₄, Cl, Br, 1 or HSO₄.

155. The method of embodiment 129, wherein 1,2-dithiol-3-ylideneammonium derivatives have the FIG. 7 formula.

156. The method of embodiment 155, wherein another group of compounds is formed in which Xθ represents a pharmaceutically acceptable anion, R represents a straight- or branched-chain alkyl radical containing 1 to 7 carbon atoms [unsubstituted-or substituted by hydroxy, carboxy, alkoxycarbonyl, cyano, dialkylamino, alkylcarbonyl, benzoyl, thenoyl, pyridyl, carbonyl, carbamoyl, dialkylcarbamoyl (the alkyl radicals of which can together form, with the nitrogen atom to which they are attached, a 5- or 6-membered heterocyclic ring optionally containing another hetero-atom selected from oxygen, sulphur, and nitrogen substituted by an alkyl or alkylcarbonyl radical) or pyridyl radical], a dialkylcarbamoyl radical (the alkyl radicals of which can together form, with the nitrogen atom to which they are attached, a 5- or 6-membered heterocyclic ring optionally containing another hetero-atom selected from oxygen, sulphur, and nitrogen substituted by an alkyl or alkylcarbonyl radical), an alkenyl radical containing 2 to 6 carbon atoms or an alkynyl radical containing 2 to 6 carbon atoms, and either R₁ and R₂, which have the same or different significances, each represent a phenyl radical, a cycoalkyl radical containing 3 to 7 carbon atoms, or an alkyl or phenylalkyl radical or alternatively together form, with the nitrogen atom to which they are attached, a 5-, 6- or 7-membered heterocyclic ring which can optionally contain another hetero-atom selected from oxygen, sulphur, and nitrogen substituted by an alkyl radical, or R₁ represents a phenyl radical a cycloalkyl radical containing 3 to 7 carbon atoms, or an 1 alkyl or phenylalkyl radical, and R₂ represents a hydrogen atom, and also the corresponding bases when R₂ represents hydrogen, the aforementioned alkyl and alkoxy radicals and moieties containing 1 to 4 carbon atoms in a straight or branched-chain unless otherwise mentioned.

157. The method of embodiment 155, wherein another group of compounds is formed in which Xθ represents a pharmaceutically acceptable anion, R represents an alkenyl radical containing 2 to 6 carbon atoms, or a straight- or branched-chain alkyl radical containing 1 to 7 carbon atoms [unsubstituted or substituted by a cyano, dialkylamino, carbamoyl, alkylcarbonyl or thenoyl radical, or a benzoyl radical the phenyl ring of which is unsubstituted or substituted by one or more halogen atoms or radicals selected from alkyl, alkoxy, hydroxy and, cyanol, the aforementioned alkyl and alkoxy radicals and moieties containing 1 to 4 carbon atoms in a straight- or branched-chain unless otherwise stated, and R₁ and R₂ together-with the nitrogen atom to which they are attached represent a pyrrolidin-1-yl or morpholino radical.

158. The method of embodiment 155, wherein another group of compounds is formed in which Xθ represents a pharmaceutically acceptable anion, R represents a methyl or ethyl radical unsubstituted or substituted by a benzoyl radical the phenyl ring of which is unsubstituted or substituted by one or more halogen atoms or radicals selected from alkyl and alkoxy radicals containing 1 to 4 carbon atoms in a straight- or branched-chain, and the hydroxy and cyano radical and R₁ and R₂ together with the nitrogen atom to which they are attached represent the morpholino radical.

159. The method of embodiments 155 to 158, wherein 1,2-dithio-1-3-ylideneammonium derivatives can be selected from the group comprising:

N-[5-(4-chlorophenacylthio)-1 2-dithiol-3-ylidene] morpholinflum chloride;

N-[5-(3-methoxyphenacylthio)-1,2-dithiol-3-ylidene]-morpholinium chloride;

N-[5-(4-fluorophenacylthio)-1,2-dithiol-3-ylidene]-morpholinium chloride;

N-[5-(2,4-dichlorophenacylthio)-1,2-dithiol-3-ylidene]-morpholinium chloride;

N-[5-(2-chlorophenacylthio)-1,2-dithol-3-ylidene]-morpholinium iodide;

N-[5-(4-hydroxyphenacylthio)-1,2-dithiol-3-ylidene]-morpholinium chloride;

N-[5-(4-methoxyphenacylthio)-1,2-dithiol-3-yllidene]-morpholinium iodide;

N-[5-(4-methylphenacylthio)-1,2-dithiol-3-ylidene]-morpholinium chloride;

N-[5-(4-cyanophenacylthio)-1,2-dithiol-3-ylidene]-morpholinium chloride; and

N-[5-(phenacylthio)-1,2dithiol-3-ylideene]-morpholinium chloride.

160. The method of embodiment 103, wherein the compounds of the present invention are isobenzothiazolone derivatives which include FIG. 24 compounds.

161. The method of embodiment 160, wherein R¹ is nitro or arylazo and R² as hydrogen.

162. The method of embodiment 160, wherein R² is hydrogen and R¹ is phenylazo; substituted arylazo such as 4-hydroxyphenylazo; 4-nitro-2-methylphenylazo; 2-hydroxy-l-napthylazo; 2-hydroxy-5-methylphenylazo; 2-hydroxy-4-methyl-5-nitrophenylazo; 4-hydroxy-l-napthylazo; 4-hydroxy-3-methyl-l-napthylazo; 4-hydroxy-5-aza-1-napthylazo; 2-amino-l-napthylazo; 1-hydroxy-2-napthylazo; 3-N,N-dimethylaminopropylcarboxyamido-1-hydroxy-4-naph-thylazo; 1-hydroxy-4-methoxy-2-naphthylazo, 2-hydroxy-3-carboxy-l-naphthylazo; 1-hydroxy-3, 6 disulfonato-2-naphthylazo; 2,3-dihydroxy-l-naphthylazo; or 2-hydroxy-3, 5-dimethyl-1-phenylazo. In one particular embodiment R¹ is the substituted ben zylideneamino, 2,4-dinitrobenzylideneamino and R² is hydrogen. Additionally R¹ as hydrogen and R² as 2-hydroxy-l-naphthylazo or 4-hydroxy-lphenylazo.

163. The method of embodiment 160, wherein R³ may be —(CH₂)nR⁴R⁵ where n is from 2 to 6, 3-carboxypropyl, sulfonatoethyl and polyethyl ethers of the type —CH₂(CH₂OCH₂),CH₃ where n is less than 10 and R⁴ and R⁵ are simple alkyls or hydrogens.

164. The method of embodiment 163, wherein R³ side chains are aminoalkyl, carboxyalkyl, omega amino polyethyl ethers, N-haloacetyl derivatives, alkyl, aryl, heteroaryl, alkoxy, hydroxy, amino groups, aminoalkyl, carboxyalkyl, hydroxyalkyl or haloalkyl, aminoaryl, carboxyryl or hydroxyaryl groups.

165. The method of embodiment 103, wherein the compounds of the present invention are isobenzothiazolone derivatives with the FIG. 25 structure:

166. The method of embodiment 165, wherein the R³ substituents comprise an alkyl linked to an amine bearing polymer by amine displacement of a halogen from an alpha-haloalkyl or alpha-haloalkylcarbox amido R³ precursor. In the case of aminoalkyl or aminoaryl groups the R³ substituent may also be covalently linked to a polymer such as polyepichlorohydrin, chloromethylpolystyrene, polyvinylalcohol or polyvinylpyridine. The R³ substituent of the present invention may generally be an aminoalkyl, hydroxyalkyl, aminoaryl or hydroxyary] group linked to a polymer comprising carboxyl groups through amide or ester linkages.

167. The method of embodiment 165, wherein when polymers are involved in the R³ structure, the polymer may be one such as polyacrylic acid, polymethacrylic acid, polyitaconic acid, oxidized polyethylene oxide, poly (methylmethacrylate/methacrylic acid), carboxyinethyl cellulose, carboxymethyl agarose or carboxymethyl dextran. When such a carboxyl polymer is involved, the R³ may be aminoalkyl (such as 8 aminohexyl, for example), hydroxyalkyl, aminoaryl or hydroxyaryl linked to the polymer through amide or ester linkages. In such cases, an R³ precursor function may bear an amine or hydroxyl group to be covalently linked to a polymer by reaction with an acid anhydride bearing polymer or by coupling with a carboxylate bearing polymer through carbodimide induced bond formation.

168. The method of embodiment 165, wherein the R³ substituent or precursor thereto in the compound of the present invention may also be a haloalkyl or carboxylialoalkyl moiety such as chloracetamido. Such a substituent may readily coupled to an amine bearing polymer by amine displacement of the halogen.

169. The method of embodiment 103, wherein the compounds are further selected from the FIG. 23 compounds.

170. The method of embodiment 169, wherein R₁ and R₂ together form a mono- or polycyclic C₂-C₂₀ alkylene group optionally comprising one or more hetero atoms.

171. The method of embodiment 169 wherein, R is chosen from a C₁-C₆, alkyl group, and their pharmaceutically acceptable salts.

172. The method of embodiment 169, wherein the aryl group or aryl fraction of an arylalkyl group denotes an aromatic carbon-based group such as a phenyl or naphthyl group or an aromatic heterocyclic group such as a thienyl of furyl group, it being possible for these groups to bear one or more substituents chosen from a halogen atom, a C₁-C₄ alkyl group, a C₁-C₄ alkoxy group, a trifluoromethyl group, a nitro group and a hydroxyl group.

173. The method of embodiment 103, wherein the compounds are further selected from the FIG. 26 and FIG. 27 compounds.

174. The method of embodiment 173, wherein the compound can be selected from the group comprising: 3-keto lipoic acid, 3-hydroxy lipoic acid, 3-keto dihydrolipoic acid or 3-hydroxy dihydrolipoic acid.

175. The method of embodiment 103, wherein the compounds are selected from the group comprising compounds of FIG. 29, FIG. 30, FIG. 31, FIG. 31a, FIG. 32 and FIG. 33.

176. The method of embodiment 103, wherein the compounds are FIG. 36 or FIG. 37 compounds.

177. The method of embodiment 176, wherein another group of compounds is formed in which Y is nitro and n is 1.

178. The method of embodiment 176, wherein another group of compounds is formed in which Y is trifluoromethyl and n is 1.

179. The method of embodiment 176, wherein another group of compounds is formed in which Y is trifluoromethyl and n is 2.

180. The method of embodiment 176, wherein another group of compounds is formed in which Y is nitro and n is 2.

181. The method of embodiment 176, wherein another group of compounds is formed in which Y is CF₃ and n is 2.

182. The method of embodiment 176, wherein another group of compounds is formed in which Y is CF₃ and n is 2.

183. The method of embodiment 176 to 182, wherein the compound is:

S-tertbutyl-S′-(2,4-dinitro-3-aminopropyl-6-tri-fluoromethylphenvl)-trithio-carbonate.

184. The method of embodiment 103 wherein the compounds are compounds given for FIG. 38, wherein R is H or a C₁ to C₁₂ alkyl moiety; R₁ is a C₆ to C₁₂ arylene moiety; R₂ is a C₁ to C₄ alkylene moiety; and n is 2 to 50.

185. The method of embodiment 103 wherein, the compounds are compounds given for FIG. 39 wherein the dotted line is optionally present and wherein the groups R₁ and R₂ are independently selected from the group consisting of hydrogen; C₁₋₂₀ alkyl groups and C₂₋₁₂ alkenyl groups.

186. The method of embodiment 185, wherein another group of compounds is formed in which R₁ and R₂ are independently selected from the group consisting of hydrogen, C₁₋₄ alkoxy groups, and C₂₋₄ alkenyl groups.

187. The method of embodiment 185, wherein another group of compounds is formed in which R₁ and R₂ are each hydrogen.

188. The method of embodiment 103 wherein the compounds is a compound of FIG. 40, wherein R and R1 are the same or different and each is an alkyl radical having from 1 to 12 carbon atoms, a cycloalkyl radical having from 5 to 12 carbon atoms which may be substituted with alkyl groups having from 1 to 4 carbon atoms or an aralkyl radical having from 7 to 14 carbon atoms, and Y is hydrogen, mercapto or SW where R′ is an alkyl radical having from 1 to 20 carbon atoms, cycloalkyl having from 5 to 12 carbon atoms, alkenyl from 3 to 20 carbon atoms, or aralkyl having from 7 to 14 carbon atoms.

189. The method of embodiment 188, wherein another group of compounds is formed in which R and R1 are branched-chain alkyl radicals having from 3 to 8 carbon atoms,1-methyl cyclohexyl or aa-dimethyl benzyl.

190. The method of embodiment 188, wherein another group of compounds is formed in which Y is an —S-alkyl group having from 6 to 18 carbon atoms.

191. The method of embodiments 188 to 190, wherein the compounds are selected from the group comprising:

4-(3,5-di-isopropyl-4-hydroxyphenyl)-1,2-dithiole-3-thione;

4-(3,5-di-t-butyl-4-hydroxyphenyl)-1,2-dithiole-3-thione;

4-[3,5-bis(l,l-dimethylpropyl)-4-hydroxyphenyl]-1,2-dithi-ole-3-thione;

4-[3,5bis(l,l-dimethylbutyl)-4-hydroxyphenyl]-1,2-dithiole-3-thione;

4-[3,5-bis(1,1,3,3-tetramethylbutyl)-4-hydroxyphenyl]-1,2-dithiole-3-thione;

4-[3,5-bis(l-methylcyclohexyl)-4-hydroxyphenyl]-1,2-dithiole-3-thione;

4-[3,5-bis(l,l-dimethylbenzyl)-4-hydroxyphenyl]-1,2-dithiole-3-thione;

4-(3t-butyl-4-hydroxy-S-isopropylphenyl)-1,2-dithiole-3-thione;

4-(3t-butyl-4-hydroxy-5-methylphenyl)-1,2-dithiole-3-thione;

4-[3(1,1-dimethylpropyl)-4-hydroxy.-5-isopropylphenyl]-1,2-dithiole-3-thione;

4-[3(1,1-dimethylbenzyl)-4-hydroxy-5-isopropylphenyl]-1,2-dithiole-3-thione;

5-benzylthio-4-(3,5-di-t-butyl-4-hydroxyphenyl)-1,2-dithiole-3-thione;

5-benzylthio-4-[3,5-bis(l,l-dimethylpropyl)-4-hydroxy-phenyl]-1,2-dithiole-3-thione;

5-hexylthio-4-(3,5-di-t-butyl-4-hydroxyphenyl)-1,2-dithole-3-thione;

5-hexylthio-4-[3,5-bis(l,l-dimethylbutyl)-4-hydroxy-phenyl]-1,2-dithiole-3-thione;

5-octadecylthio-4-(3,5-di-t-butyl-4-hydroxyphenyl)-1,2-dithiole-3-thione;

5-octadecylthio-4-[3,5 bis(l,l-dimethylbenzyl)-4-hydroxyphenyl]-1,2-dithiole-3-thione;

5-allylthio-4-(3,5-di-t-butyl-4-hydroxyphenyl)-1,2-dithiole-3-thione;

5-cyclohexylthio-4-(3,5-di-t-butyl-hydroxyphenyl)-1,2-dithiole-3-thione; and

4-(3,5-di-sec -butyl-4-hydroxyphenyl)-1,2-dithiole-3-thione.

192. The method of embodiment 188, wherein another group of compounds is formed in which Y is the residue of a FIG. 41 compound.

193. The method of embodiment 103, wherein the compounds of the present invention have FIG. 42 formula, wherein A is a methylene group or an oxygen atom; R′ and R2 are independently a hydrogen atom, a hydroxyl group, a halogen atom, a lower alkyl group or a lower alkoxy group; and n is an integer of 0-3 when A is a methylene group, and an integer of 1-3 when A is an oxygen atom; or a salt thereof.

194. The method of embodiment 193, wherein another group of compounds is formed in which A is a methylene group and R2 is a hydrogen atom; or a salt thereof.

195. The method of embodiment 193, wherein another group of compounds is formed in which R′ is a hydrogen atom, a hydroxyl group or a lower alkoxy group; or a salt thereof.

196. The method of embodiment 193, wherein another group of compounds is formed in which A is an oxygen atom and R2 is a hydrogen atom; or a salt thereof or alternatively R, is a hydrogen atom, a hydroxyl group or a lower alkoxy group; or a salt thereof.

197. The method of embodiments 193 to 196, wherein of the following compounds (a) to (k); or a salt thereof.

198. The method of embodiments 193 to 197, wherein the compound has the FIG. 43 formula wherein k is an integer of 0-5; X and Y are independently a hydrogen atom, a lower alkyl group or a lower alkoxy group; R¹¹ is an alkyl group or —(CH₂)ₘ—C₆H₂—R12R13R14, wherein m is an integer of 0-4; and R12, R13 and R14 are independently a hydrogen atom, a lower alkyl group or a lower alkoxy group; however, a case is excluded in which both k and m are zero, the suffo group bonds to the 3-position, X is a 4-methoxy group, and R12, R13, R14, and Y are each a hydrogen atom); or a salt thereof.

199. The method of embodiment 198, wherein another group of compounds is formed in which R¹¹ is an alkyl group; or a salt thereof.

200. The method of embodiment 198, wherein another group of compounds is formed in which R¹¹ is —(CH₂)ₘ—C₆H₂—R12R13R14.

201. The method of embodiments 198 to 200 wherein the following compounds; or a salt thereof can be selected from the group comprising:

5-Hexyl-4-(4-methoxy-3-suifobenzyl)-3H-1,2-dithole-3-thione; and

4-(4-Methoxy-3-suifophenyi)-5-(p-toiyi)-3H-1,2-dithiole-3-thione.

202. The method of embodiment 104 wherein, the D-amino acids oxidase inhibitors are further selected from the group comprising: 2-oxo-3-pentynoate; acetylacetonate and kojic acid.

203. A pharmaceutical formulation for treating patients having toxic amounts of metal in the body or in certain body compartments, comprising administering to a patient in need thereof a prophylactically or therapeutically effective amount of a composition selected from the group comprising oltipraz, 1,2-dithiole-3-thione, 1,3-dithiole-2-thione, 1,2-dithiolane, [1,2]Dithiolo[4,3-c]-1,2-dithiole-3,6-dithione, malotilate, ADT, and ADO.

204. A pharmaceutical formulation for treating patients having toxic amounts of metal in the body or in certain body compartments, comprising administering to a patient in need thereof a prophylactically or therapeutically effective amount of oltipraz.

205. A pharmaceutical formulation for treating Alzheimer's disease, comprising administering to a patient in need thereof a prophylactically or therapeutically effective amount of a composition selected from the group comprising oltipraz, 1,2-dithiole-3-thione, 1,3-dithiole-2-thione, 1,2-dithiolane, [1,2]Dithiolo[4,3-c]-1,2-dithiole-3,6-dithione, malotilate, ADT, and ADO.

206. A pharmaceutical formulation for treating Alzheimer's disease, comprising administering to a patient in need thereof a prophylactically or therapeutically effective amount of oltipraz.

207. A pharmaceutical formulation for memory enhancement, comprising administering to a patient in need thereof a prophylactically or therapeutically effective amount of a composition selected from the group comprising oltipraz, 1,2-dithiole-3-thione, 1,3-dithiole-2-thione, 1,2-dithiolane, [1,2]Dithiolo[4,3-c]-1,2-dithiole-3,6-dithione, malotilate, ADT, and ADO.

208. A pharmaceutical formulation for memory enhancement, comprising administering to a patient in need thereof a prophylactically or therapeutically effective amount of oltipraz.

209. A pharmaceutical formulation for treating malaria or a trypanosome infection, comprising administering to a patient in need thereof a prophylactically or therapeutically effective amount of a composition selected from the group comprising oltipraz, 1,2-dithiole-3-thione, 1,3-dithiole-2-thione, 1,2-dithiolane, [1,2]Dithiolo[4,3-c]-1,2-dithiole-3,6-dithione, malotilate, ADT, and ADO.

210. A pharmaceutical formulation for treating malaria or a trypanosome infection, comprising administering to a patient in need thereof a prophylactically or therapeutically effective amount of oltipraz.

211. A pharmaceutical formulation for treating reducing the level of iron and/or copper in the cells of living subjects, comprising administering to a patient in need thereof a prophylactically or therapeutically effective amount of a composition selected from the group comprising oltipraz, 1,2-dithiole-3-thione, 1,3-dithiole-2-thione, 1,2-dithiolane, [1,2]Dithiolo[4,3-c]-1,2-dithiole-3,6-dithione, malotilate, ADT, and ADO.

212. A method to reduce the level of iron and/or copper in the cells of living subjects or for chelating iron or copper ions in a mammal, comprising administering to a mammal or patient in need thereof a prophylactically or therapeutically effective amount of a compound of the invention, a compound of FIG. 1-FIG. 4, oltipraz or a compound described in any of the foregoing numbered embodiments.

213. Use of an effective amount of a D-amino acid oxidase inhibitor to treat or prevent a neurodegenerative disorder or a neurodegenerative-related disorder comprising administering to a mammal in need thereof an effective amount of the D-amino acid oxidase inhibitor.

214. Use of embodiment 213 wherein the D-amino acid oxidase inhibitor is a compound of the invention, e.g., a compound of FIG. 1-FIG. 4, oltipraz or a compound described in any of the foregoing numbered embodiments, a composition described in any of the following numbered embodiments.

215. A composition comprising a pharmaceutically acceptable carrier and a compound of the formula

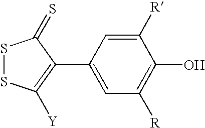

wherein R and R′ independently are the same or different and each is C1-C12 alkyl or C5-C12 cycloalkyl, either of which are optionally substituted with C1-C4 alkyl or C7-C14 aralkyl; and

Y is —H, —SH or —SR² where R² is C1-C20 alkyl radical, C5-C12 cycloalkyl, C3-C20 alkenyl, or C7-C14 aralkyl.

216. The composition of embodiment 215 wherein

(1) R and R¹ are branched-chain alkyl radicals having from 3 to 8 carbon atoms, 1-methyl cyclohexyl or αα-dimethyl benzyl;

(2) Y is an —S-alkyl group having from 6 to 18 carbon atoms; or

(3) the compound is 4-(3,5-di-isopropyl-4-hydroxyphenyl)-1,2-dithiole-3-thione, 4-((3,5-di-t-butyl-4-hydroxyphenyl)-1,2-dithiole-3-thione, 4-[3,5-bis(1,1-dimethylpropyl)-4-hydroxyphenyl]-1,2-dithiole-3-thione, 4-[3,5-bis(1,1-dimethylbutyl)-4-hydroxyphenyl]-1,2-dithiole-3-thione, 4-[3,5-bis(1,1,3,3-tetramethylbutyl)-4-hydroxyphenyl]-1,2-dithiole-3-thione, 4-[3,5-bis(1-methylcyclohexyl)-4-hydroxyphenyl]-1,2-dithiole-3-thione, 4-[3,5-bis(1,1-dimethylbenzyl)-4-hydroxyphenyl]-1,2-dithiole-3-thione, 4-(3-t-butyl-4-hydroxy-5-isopropylphenyl)-1,2-dithiole-3-thione, 4-(3-t-butyl-4-hydroxy-5-methylphenyl)-1,2-dithiole-3-thione, 4-[3-(1,1-dimethylpropyl)-4-hydroxy-5-isopropylphenyl]-1,2-dithiole-3-thione, 4-[3-(1,1-dimethylbenzyl)-4-hydroxy-5-isopropylphenyl]-1,2-dithiole-3-thione, 5-benzylthio-4-3,5-di-t-butyl-4-hydroxyphenyl)-1,2-dithiole-3-thione, 5-benzylthio-4-[3,5-bis(1,1-dimethylpropyl)-4-hydroxy-phenyl]-1,2-dithiole-3-thione, 5-hexylthio-4-(3,5-di-t-butyl-4-hydroxyphenyl)-1,2-dithiole-3-thione, 5-hexylthio-4-[3,5-bis(1,1-dimethylbutyl)-4-hydroxy-phenyl]-1,2-dithiole-3-thione, 5-octadecylthio-4-(3,5-di-t-butyl-4-hydroxyphenyl)-1,2-dithiole-3-thione, 5-octadecylthio-4-[3,5-bis(1,1-dimethylbenzyl)4-hydroxyphenyl]-1,2-dithiole-3-thione, 5-allylthio-4-(3,5-di-t-butyl-4-hydroxyphenyl)-1,2-dithiole-3-thione, 5-cyclohexylthio-4-(3,5-di-t-butyl-4-hydroxyphenyl)-1,2-dithiole-3-thione or 4-(3,5-di-sec-butyl-4-hydroxyphenyl)-1,2-dithiole-3-thione.

217. A composition comprising a pharmaceutically acceptable carrier and a complex comprising a metal ion and a compound having the formula

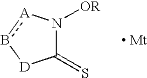

wherein

Mt is a copper ion or a metal ion of Group VIII or IIB of the Periodic Table;

A and B independently are —C(R²)₂—, —CR²═ or >C═NR²;

D is —NR²— or —S—;

R is —H;

R² is —H, C₁₋₆ hydrocarbyl optionally substituted by 1, 2 or more halogens or, two R² groups together with the carbon atom or carbon atoms to which they are attached comprise 5- or 6-membered saturated or unsaturated hydrocarbon ring system; and

the dotted line is represents a optional double bond.

218. The composition of embodiment 217 wherein

(1) A and B are —CR²═ and D is —S—;

(2) the metal ion is iron, copper or zinc;

(3) the compound is 3-hydroxy-4-methylthiazol-2(3H)-thione, 3-hdyroxy4-phenylthiazol-2(3H)-thione, 3-hydroxy-4,5,6,7-tetrahydrobenzothiazol-2(3H)-thione, 5,5-dimethyl-1-hydroxy-4-imimo-3-phenylimidazolidine-2-thione, 1-hydroxy4-imino-3-phenyl-2-thiono-1,3-diazaspiro[4,5] decane, 4,5-dimethyl-3-hydroxythiazol-2(3H)-thione, 4-ethyl-3-hydroxy-5-methylthiazol-2(3H)-thione, 4-(4-chlorophenyl)-3-hydroxythiazol-2(3H)-thione, 3-hydroxy-5-methyl-4-phenylthiazol-2(3H)thione, 1-hydroxy-5-methyl-4-phenyl imidazoline-2-thione, or 3-hydroxy-5-methyl-4-phenylthizol-2(3H)-thione;

(4) the metal ion is a zinc ion and the compound is 3-hydroxy4-methylthizole-2(3H)-thione, 4,5-dimethyl-3-hydroxythiazole-2(3H)-thione or 4-ethyl-3-hydroxy-5-methylthiazole-2(3H)-thione;

(5) R² is —H, methyl, ethyl, phenyl or chlorophenyl;

(6) A and B are linked through a double bond.

219. A pharmaceutical composition comprising a pharmaceutically acceptable carrier and a compound of the formula

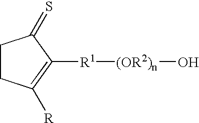

wherein R is —H or C₁ to C₁₂ alkyl; R¹ is C₆ to C₁₂ arylene; R² is C₁ to C₄ alkylene; and n is 2 to 50, e.g., 2,3, 4, 5 or 6.

220. A pharmaceutical composition comprising a pharmaceutically acceptable carrier and a compound of formula I or II

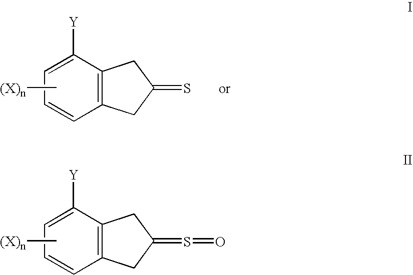

wherein

Y nitro or trifluoromethyl;

X is C1-C6 alkyl or C2-C6 alkenyl, nitro, trichloromethyl, trifluoromethyl, trifluromethoxy, trifluoromethylthio, trifluoromethyl-sulfoxyl, trifluoromethylsulfonyl, metoxymethyl, cyano, carboxy, halogen (F, Cl, Br, I), hydroxy, acetylamino, amino, N-phenylamino, N,N-diallylamino, N-morpholino, N-piperidino, N-piperazino, N-pyrrolidino, dimethylaminodithiocarbamyl,

or X is alkoxy, carboalkoxy, alkylthio, mono- or dialkylamino, N-alkyl-carbamyl, N,N-dialkylcarbamyl, alkylsulfoxy, alkylsulfonyl, wherein the alkyl groups comprise 1-4 carbon atoms;

n is 1, 2 or 3;

provided that at least one X is N-morpholino, N-piperidino, N-piperazino or N-pyrrolidino; and salts thereof.

221. The composition of embodiment 220 wherein

(1) the compound has formula I, Y is —NO₂ and n is 1;

(2) the compound has formula I, Y is —CF₃ and n is 1;

(3) the compound has formula I, Y is —NO₂ and n is 2;

(4) the compound has formula II, Y is —CF₃ and n is 2; or

(5) the compound is S-tert.butyl-S′-(2,4-dinitro-3-aminopropyl-6-tri-fluoromethylphenyl)-trithiocarbonate.

222. A pharmaceutical composition comprising a pharmaceutically acceptable carrier and a compound of formula

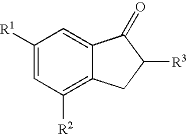

wherein

at least one of R¹ and R² is arylazo; 4-hydroxy-phenylazo; 4-nitro-2-methylphenylazo; 2-hydroxy-1-naphthylazo; 2-hydroxy-5-methylphenylazo; 2-hydroxy-4-methyl-5-nitrophenylazo; 4-hydroxy-1-naphthylazo; 4-hydroxy-3-methyl-1-naphthylazo; 4-hydroxy-5-aza-1-naphthylazo; 2-amino-1-naphthylazo; 1-hydroxy-2-naphthylazo; 3-N,N-dimethylamino-propylcarboxyamino-1-hydroxy-4-naphthylazo; 1-hydroxy-4-methoxy-2-naphthylazo; 2-hydroxy-3-carboxy-1-naphthylazo; 1-hydroxy-3,6-disulfonato-2-naphthylazo; 2,3-dihydroxy-1-naphthylazp; or 2-hydroxy3,5-dimethyl-1-phenylazo; and R¹ or R² or neither is hydrogen; and

R³ is alkyl, carboxyalkyl, hydroxyalkyl, aminoalkyl, haloalkyl, aryl, careboxyaryl, hydroxyaryl, or aminoaryl, wherein the ankyl moieties comprise 1, 2, 3, 4, 5, 6 or more carbon atoms or,

R³ is a heterocyclic radical selected from the group consisting of pyridyl, oxazolyl, quinolyl and thiazolyl any of which are unsubstituted or substituted by 1 or more carboxy, hydroxy or amino, hydroxyl, alkoxy or amino.

223. The composition of embodiment 222 wherein

(1) R¹ is 4-hydroxyphenylazo, and R² is —H;

(2) R¹ is phenylazo, and R² is —H;

(3) R¹ is 2-hydroxy-1-naphthylazo and R² is —H;

(4) R¹ is 2-hydroxy-5-methylphenylazo and R² is —H;

(5) R¹ is 4-hydroxy-1-naphthylazo, and R² is —H;

(6) R¹ is 4-hydroxy-3-methyl-1-naphthylazo, and R² is —H;

(7) R¹ is 4-hydroxy-5-aza-1-naphthylazo, and R² is —H;

(8) R¹ is 2-amino-1-naphthylazo, and R² is —H; or

(9) R¹ is 1-hydroxy-2-naphthylazo, and R² is —H.

224. Use of any of the compounds or complexes disclosed in the compositions of embodiments 215-223 to prepare a medicament for use in treating or to slow the progression of a neurodegenerative disorder, a neurodegenerative-related disorder, malaria or a trypanosome infection or any of the other conditions or infections disclosed herein.

225. The method or use of any of the foregoing embodiments or the following claims that recite treating, preventing, ameliorating a symptom(s) of, or slowing progression of (or the like) a degenerative or neurodegenerative or related disorder, whereby the administration of the compound of the invention to a subject treats, prevents, ameliorates a symptom(s) of, or slows the progression of the degenerative or neurodegenerative or related disorder.

Whereas, particular embodiments of this invention have been described above for purposes of illustration, it will be evident to those skilled in the art that numerous variations of the details of the present invention may be made without departing from the invention as defined in the appended claims. The invention is not limited to the embodiments described above, but may be varied in both construction and detail within the scope of the claims.

All publications and references cited herein are incorporated herein by reference.

The invention will be more clearly understood from the following description of some embodiments of the invention, which are not to be interpreted as limiting the scope of the claimed invention in any way.

### EXAMPLE 1

Effect of Oltipraz on Aβ1-42 Neurotoxicity In Vitro.

Primary cortical neuron cultures were established from E14 mice. On day 6 in vitro, medium was replaced with Neurobasal/B27 (without antioxidants) and neurons were treated with Oltipraz (optimally 20 μM) and Aβ1-42 (25 μM). Cultures were incubated for 5 days (37° C.) and then cell viability was determined using the MTT reduction assay. Cultures were also monitored for morphological changes by phase contrast microscopy. The MTT assay was performed by adding water soluble MTT to the culture medium at 0.5 mg/mL and incubating for 30 min. (Longer incubations lead to non-specific inhibition of MTT uptake by Aβ). The culture medium was removed and the MTT solubilized in DMSO. Aliquots of solubilized MTT were read on a spectrophotometric plate reader at 570 nm.

Results: Oltipraz (20 μM) n the presence of Aβ1-42 (25 μM) resulted in a small increase in cell viability (p<0.05, ANOVA) compared to Aβ1-42 alone.

### EXAMPLE 2

Effect of Oltipraz on a Murine Model for Oxidative Stress.

Primary fibroblast cultures were prepared from normal mice (non Tg), mice transfected to produce human wild type presenilin 1 (WT Tg) or mice transfected to produce mutant human presenilin 1 (Mut Tg). All mice are derived from an identical genetic background. Cells were treated with H₂O₂ (150 μM) with or without Oltipraz (25 μM, optimal concentration from DMSO stock). 50U/mL of catalase was used as a benchmark antioxidant against H₂O₂ toxicity. Cell viability was determined with the MTT reduction assay. In additional experiments, Mut Tg fibroblasts were pre-treated with the glutathione synthesis inhibitor, buthionine sulfoximine (BSO) with or without Oltipraz (10, 25 or 50 μM). Cell viability was determined with the MTT assay.

Results: Oltipraz had a significant protective effect against H₂O₂ toxicity in fibroblasts from non Tg and WT Tg mice but not in fibroblasts from Mut Tg mice. The protective effect was about two thirds of that achieved with 50U/mL catalase. Oltipraz, 10, 25 and 50 μM was effective in restoring cell viability to 100% in fibroblasts treated with toxic levels of BSO. These findings confirm the role of oltipraz as an antioxidative agent.

### EXAMPLE 3

Removal of Iron from Tissues.

The ability of Oltipraz to selectively remove the reactive iron which accumulates in AD brain was compared with the ability of the known iron chelator, deferoxamine (“DFX”) and dH₂O to remove iron from AD brain tissue in vitro. AD brain sections were treated with Oltipraz or DFX for either 2 hrs or overnight. Although not as effective as DFX, oltipraz was able to remove iron from the background tissue and AD pathological lesions, especially after an overnight incubation. This analysis demonstrates that oltipraz is a potent metal chelator that is able to effectively remove redox-active iron from brain sections taken from individuals with Alzheimer disease.

Thus, oltipraz could remove redox-active transition metals from AD brain sections. Given that there is little in vivo toxicity of the oltipraz when it is used in a therapeutic setting, these data suggest that abnormally localized iron found in the disease can arise as opposed to a total removal of all cellular iron. This observation is supported by our preliminary data showing little/no neurotoxicity in vitro using doses of oltipraz that are effective at chelating in situ or abolishing amyloid-β toxicity.

Attenuation of Amyloid-β Toxicity.

Oltipraz was able to significantly attenuate the neurotoxicity of amyloid-β, a key pathogenenic protein involved AD, which indicates its usefulness as an early therapeutic/preventative agent. Oltipraz increase cell survival in brain tissue sections as shown below.

[image]

It has also been surprisingly found that the toxicity of amyloid-β is mediated by iron in that toxicity was attenuated in a dose-dependent fashion by deferoxamine and restored, again in a dose-dependent fashion, by subsequent exogenous addition of ferrous iron. Notably, in vivo there is an extremely close relationship between iron and amyloid-β in the diseased brain.

It has been also found that when pre-incubated with amyloid-β, oltipraz has the ability to attenuate its toxicity in a dose-dependent manner similar to that of deferoxamine. Coupled together with the in situ demonstration that oltipraz has the ability to function as a chelator of redox-active iron from sites of iron deposition in vivo, these findings strongly support the use of oltipraz in chelation therapeutics for AD. Based on these results, the mechanisms involved in neuroprotection are believed to center, at least in part, on the fact that oltipraz is able to effectively chelate iron.

### EXAMPLE 4

Localization of 8-Hydroxyguanosine.

8-Hydroxyguanosine (8 OHG) is a nucleic acid modification predominantly derived from .OH attack of guanidine. 8 OHG is likely to form at the site of .OH production, a process dependent on redox-active metal catalyzed reduction of H₂O₂ with cellular reductants such as ascorbate or O₂⁻. Staining of cells in vitro using immunogold analysis using a 8 OHG monoclonal antibody reveals that nucleic acid oxidation is most prominent in the cytoplasmic compartment compared e.g., to 8 OHG in mitochondria or mitochondrial derived lysosomes. This is consistent with the observation that most oxidative damage in AD is cytoplasmic.

Mitochondrial Abnormalities and Oxidative Damage.

Mitochondria are a source of oxidative radicals and oxidative precursors, in the form of O₂⁻ and H₂O₂, respectively. However, mitochondria probably are not directly the source of oxidizing radicals and their role may be to supply H₂O₂, a freely diffusible precursor and through their turnover of redox-active metals. By in situ hybridization analysis with a chimeric cDNA probe to the 5 kb common deletion, it was found that deleted mtDNA was increased at least 3 fold for AD neuronal tissue compared to control tissue. Quantitative analysis of the mtDNA deletion and 8 OHG in the same cases, demonstrated a strong positive correlation (r=0.934, p=0007) as shown in the plot below.

[image]

However, given that mitochondrial DNA, even that containing the 5 kb deletion, is relatively spared in comparison to cytoplasmic nucleic acid (i.e., RNA), it is believed that mitochondrial abnormalities correlate, but do not directly cause, reactive oxygen species. In this regard, it is important to recognize that 8 OHG is formed by the direct attack of .OH. Such .OH have less than a 2 nm sphere of diffusion and are unable to diffuse through the mitochondrial membrane. Therefore, since damage is topographically distinct, it is likely that .OH formation occurs in the cytoplasm rather than the mitochondria. Rather, abnormal mitochondria may actually produce excess H₂O₂ through conversion of O₂⁻ by mitochondrial SOD. Such H₂O₂ is readily diffusible and relatively stable, that is until confronting redox-active transition metals at which point, Fenton chemistry drives the production of .OH. Thus, mitochondrial abnormalities appear to correlate with, but are not directly responsible for, significant oxidative damage.

### EXAMPLE 5

In Vivo Studies of Redox-Active Metal.

AD is associated with abnormalities of iron metabolism including increased levels of free iron as well as altered levels of iron transport and storage proteins. The H₂O₂-dependent oxidation of 3,3′-diaminobenzidine (DAB) was used to determine sites of non-enzymatic catalytic redox activity in tissue sections from AD and control brain tissue sections, it was directly demonstrated that at least some of the iron that is associated with AD pathology was redox active. In addition to the NFT and Aβ deposits, redox active metals can be identified in the cytoplasm by the same DAB reaction. Significantly, these structures (lipofuscin and mitochondria) were found to be redox inactive in age-matched controls.

Although in situ histochemical techniques lack the sensitivity to detect copper, copper (as well as iron) could contribute to the redox activity. The relative effectiveness of copper- and iron-selective chelating agents was used to remove the lesion-dependent redox activity, which provided evidence for both copper- and iron-mediated redox activity in AD. The results showed H₂O₂-dependent oxidation of 3,3′-diaminobenzidine by the lesions in AD neuronal tissues was greatly reduced by 10 mM DFX but was completely abolished by 10 mM DTPA, indicating

Also, DAB oxidation was inhibited by chelation of metals with detapac (DTPA) or deferoxamine (DFX), with the former being more effective on an equimolar basis for neurofibrillary tangles (NFT), senile plaques (SP), and the cytoplasmic vesicles. Prior treatment of AD brain tissue sections with 100 mM or 10 mM DTPA abolished all or nearly all, respectively, lesion-associated oxidation of DAB, and 1 mM DTPA still inhibited more than half of the DAB staining. In contrast, 100 mM DFX was incompletely effective in inhibiting the lesion- and vesicle-associated DAB oxidation, 10 mM DFX reduced the DAB staining by only about half, and the inhibitory effect of 1 mM DFX was barely noticeable. Following removal of metals with 100 mM DFX, lesion-associated H₂O₂-dependent DAB oxidation could be re-established by incubation of the tissue sections either with a mixture of 0.01 mM FeCl₂ and 0.01 mM ferric citrate or with 0.01 mM CuSO₄, with copper being more effective than iron.

The results indicated that NFT, SP, and vesicles bind endogenous redox active transition metals in a manner that permits them to catalyze H₂O₂ oxidation of DAB at the site of metal binding, which implicates a cycling of the metal ions between oxidized and reduced states. There are only limited types of protein sites for adventitiously-bound metal ions expected to have sufficient affinity for both reduced and oxidized states thereof as to resist complete removal of the metals by chelators. Iron and copper were examined because they are the most common redox-active circulating metals and because the criteria for redox activity of other potential transition metals (cobalt, nickel, manganese, and chromium) are more stringent and/or typically limited to specially designed enzyme active sites.

Amyloid-β Toxicity is Mediated by Iron.

In vitro, amyloid-β is toxic to neurons and clonal cell lines. It was surprisingly found that the toxicity of amyloid-β is mediated by iron in that toxicity was attenuated in a dose-dependent fashion by deferoxamine and restored, again in a dose-dependent fashion, by subsequent exogenous addition of ferrous iron. Notably, in vivo there is an extremely close relationship between iron and amyloid-β in the diseased brain. Oltipraz was pre-incubated with amyloid-β , and the toxicity of β-amyloid was reduced in a dose-dependent manner similar to that of deferoxamine.

### EXAMPLE 6

Inhibition of Parasites In Vitro.

For in-vitro antimalarial testings, micro-titer plates were used. The concentration of drugs was prepared as pMol/well according to WHO standard procedures (WHO, 1990). The test compound was dissolved in 15% DMSO in sterile RPMI 1640. Chloroquine sensitive isolates were used throughout the experiments.

A. Schizont Inhibition Assay:

The micro-titer plates were predosed with various concentration of the test compound. 50 μL of parasitised erythrocyte suspension in RPMI-1640 (0.2 ml erythrocyte +0.3 ml serum +4-5 ml RPMI-1640) were dispensed in microtiter wells containing various concentrations of drug. Triplicate readings were made for each concentration.

B. ³H-Hypoxanthine Incorporation Assay:

The testing was carried out according to the procedure of Desjardins et al. 1979. After 30 hr culture at 37 degrees C., the same microtiter plates from schizont inhibition assays with another triplicate wells were pulsed with ³H-hypoxanthine for overnight. The cell suspensions were washed twice on millipore glass fiber filter with Millipore filter apparatus. The filter discs were counted for DPM by Beckman LS6000-scintillation counter. The activity of the drug was measured by plotting DPM against concentration of drug.

In Vitro Anti-Protozoal Activity of Oltipraz.

The results shown below demonstrate the capacity of compounds such as oltipraz to inhibit growth or replication of infectious agents such as the exemplified protozoa.

| | | | Toxi- |
|---|---|---|---|
| | | | city |
| Com- | % Inhibition | ED₅₀ | ED₅₀ |

| | | | | | | | | |
|---|---|---|---|---|---|---|---|---|
| pound | Parasite | 30 | 10 | 3 | 1 | 0.3 | μg/ml | μg/ml |

| | | | | | | | |
|---|---|---|---|---|---|---|---|
| Oltipraz | L. donovani | 3.2 | 0 | | | >30 | 52.9 |
| | T. cruzi | 99.7 | 54.6 | 38.6 | 4.3 | 6.24 | |
| | | | | | | (23.8) | |
| | T.b.rhodesiense | 42.5 | 0 | | | >30 | |
| | STIB900 | | | | | | |
| | P. falciparum | 100 | 96.5 | 4.4 | 0 | 5.7 | |
| | 3D7 | | | | | | |

Podophyllotoxin ED₅₀ 0.003 μg/mL
Pentamidine ED₅₀ 0.0001 μg/mL
Chloroquine ED₅₀ 0.005 μg/mL
Pentostam ED₅₀ 25 μg/mL
Benzoidazole ED₅₀ 1.0 μg/mL

### EXAMPLE 7

Oltipraz Synthesis.

Oltipraz was prepared in steps 1-3 as follows. Step 1 (esterification of pyrazine-2-carboxylic acid to yield methyl-pyrazine-2-carboxylate): To a 1L single-neck round-bottomed flask fitted with condenser and drying tube filled with silica gel was charged methanol (400 mL) with agitation at room temperature. Pyrazine-2-carboxylic acid (50.00 g, 402.90 mmol) was charged to the flask in one portion and the resulting slurry was vigorously stirred. Conc. sulfuric acid (0.25 mL) was charged to the slurry. The slurry was heated to reflux temperature and stirred at this temperature for 2 days. The resulting pale yellow solution was allowed to cool to room temperature. This process takes 90 minutes. Solid sodium bicarbonate (4.00 g, 47.62 mmol) was added to the solution in one portion and the slurry was stirred vigorously for 30 minutes. The suspension was filtered and the filtrate was transferred to a 2L single-neck round-bottomed flask and concentrated to about half volume in vacuo @ 35° C. Toluene (1200 mL) was added to the methanol solution and a Dean—Stark trap fitted with drying tube was attached. The solution was heated at atmospheric pressure (external oil bath @120° C.) and the first 300 mL solvent fraction was run off and discarded. The Dean-Stark trap was removed and the reaction solution was concentrated in vacuo @45° C. to a volume of 300 mL. The organic phase containing the desired methyl-pyrazine-2-carboxylate was filtered to remove solid particulates and used directly in the next step as a solution in toluene (a small analytical sample was removed and concentrated in vacuo @ 35° C. to yield a pale brown solid, m.p. 60-61° C., structure confirmed by ¹H NMR and ¹³C NMR).

Step 2 (claisen condensation of methyl-pyrazine-2-carboxylate with methyl propionate using sodium hydride as base to yield methyl-2-methyl-3-(pyrazin-2-yl)-3-oxopropionate): To a 2L 3-neck round-bottomed flask under a nitrogen atmosphere was charged NaH (22.11 g, 552.77 mmol) (60% dispersion in oil). Toluene (250 mL) was charged to the flask and the resulting slurry was stirred for 15 minutes at 20° C. The slurry was allowed to settle and the toluene removed by decantation. Additional toluene (250 mL) was added and the slurry was stirred for at 20° C. Methyl propionate (53.23 mL, 552.77 mmol) suspended in anhydrous toluene (250 mL) was added dropwise over 30 minutes. The resulting slurry was then heated to reflux temperature (external oil bath @140° C.). To the refluxing suspension was charged methyl pyrazine-2-carboxylate (54.72 g, 394.83 mmol) in anhydrous toluene (300 mL) (from step 1 of the process) dropwise over a period of 45 minutes. The reaction contents were heated at reflux temperature for 2.5 hours. The resultant dark brown slurry was allowed to cool to 20° C. Saturated ammonium chloride solution (500 mL) was charged to the slurry in one portion and the biphasic solution was vigorously stirred for 120 minutes, then agitation was stopped and the phases were allowed to separate. The dark brown-coloured lower aqueous phase (approx. 500 mL) was removed and the remaining yellow/orange-coloured upper organic phase (approx. 900 mL) was retained and combined with toluene extracts (2×175 mL) of the aqueous phase. The organic phase was filtered to remove solid particulates and concentrated in vacuo @45° C. to a volume of 400 mL to yield the desired methyl-2-methyl-3-(pyrazin-2-yl)-3-oxopropionate, which was used directly in the next reaction step. A small analytical sample was concentrated in vacuo @35° C. to yield a viscous oil. Structure was confirmed by ¹H NMR and ¹³C NMR.

Step 3 (treatment of methyl-2-methyl-3-(pyrazin-2-yl)-3-oxopropionate with phosphorus pentasulfide to form oltipraz): To a 3L 3-neck round-bottomed flask fitted with pressure-equalising dropping funnel with N₂ inlet, condenser with N₂ outlet and mechanical stirrer and under a nitrogen atmosphere was charged P₂S₅ (168.83 g, 759.58 mmol). Toluene (500 mL) was charged to the flask and the slurry was stirred at 20° C. Methyl-2-methyl-3-(pyrazin-2-yl)-3-oxopropionate in toluene (400 mL) (from step 2 of the process) was charged to the slurry in one portion. The resulting yellow slurry was heated to reflux temperature (110° C.) (external oil bath @135° C.) and stirred at this temperature for 18 hours. The resulting deep red-coloured slurry was cooled to 0-5° C., water was added and the resulting suspension brought to pH 8-8.5 by the addition of conc. ammonia solution (270 mL). The resulting biphasic solution was filtered to remove solid particulates, the black lower aqueous phase was removed and the deep red-coloured upper organic phase (approx. 1L) was retained and combined with the toluene extracts (2×400 mL) of the aqueous phase. The organic phase was dried over magnesium sulphate (30 g) and concentrated in vacuo @45° C. to a volume of 100 mL. Methanol (100 mL) was added and the resulting slurry was stirred for 20 minutes, then filtered through a sintered funnel and washed with methanol (2×20 mL). The dark red solid was dissolved in acetonitrile (approx. 400 mL) @78° C., 1.4 g of decolorizing charcoal was added, the solution was filtered and a red precipitate was formed by cooling to 0-5° C. The precipitate was filtered and washed with ice cold acetonitrile (1×40 mL) to afford Oltipraz as bright red needles, (approx. 6.5 g, 10%), m.p. 167-168° C., structure confirmed by ¹H NMR.

Synthesis of related compounds as disclosed herein are accomplished in a similar manner.

### EXAMPLE 8

Serum Hydrogen Peroxide Level Measurement.

When hydrogen peroxide is over produced in cells it causes lipid peroxidation and cellular damage. Over production can ultimately result in cellular degeneration. It is advantageous to monitor circulatory fluid (e.g., serum or spinal fluid) hydrogen peroxide levels in patients having or at risk for degenerative diseases.

A known assay to measure hydrogen peroxide levels is outlined below. The assay is based on the detection of hydrogen peroxide using 10-acetyl-3,7-dihydroxyphenoxazine. In the presence of horseradish-peroxidase, 10-acetyl-3,7-dihydroxyphenoxazine reacts with hydrogen peroxide to produce highly fluorescent resorufin. A standard curve is created by adding increasing amounts of hydrogen peroxide.

A solution containing 200 μM 10-acetyl-3,7-dihydroxyphenoxazine, 1 U/mL horseradish peroxidase and an appropriate amount of hydrogen peroxide and any D-amino acid (depending on the concentration), is prepared in 50 mM sodium phosphate buffer, pH 7.4. The solution is incubated for 30 minutes at room temperature. Fluorescence is measured using a fluorescence microplate reader using excitation at 560±10 nm and fluorescence detection at 590±10 nm. Background fluorescence is determined for a non-hydrogen peroxide control reaction and is subtracted from each value.

### EXAMPLE 9

Measurement of Serum Glutathione Reductase Levels in Circulatory Fluid Samples.

Glutathione reductase (GR) is an ubiquitous enzyme which catalyzes the reduction of oxidized glutathione (GSSG) to glutathione (GSH). Glutathione reductase is essential for the glutathione redox cycle that maintains adequate levels of reduced cellular GSH. GSH serves as an antioxidant, reacting with free radicals and organic peroxides, in amino acid transport, and as a substrate for the glutathione peroxidases and glutathione S-transferases in the detoxification of organic peroxides and metabolism of xenobiotics, respectively (Dolphin, 1989). GR levels are reduced in degenerative disorders.

Glutathione reductase catalyses the reduction of oxidised glutathione (GSSG) to glutathione (GSH):

GSSG+NADPH+H⁺→2 GSH+NADP⁺

Oxidized glutathione is reduced by a multi-step reaction in which GR is initially reduced by NADPH forming a semiquinone of FAD, a sulfur radical and a thiol. The reduced GR (GRred) reacts with a molecule of GSSG resulting in a disulfide interchange which produces a molecule of GSH and the GRed-SG complex. An electron rearrangement in GRed-SG, results in a second disulfide interchange, splitting off the second molecule of GSH and restoring the GR to the oxidized form (Massey, 1965).

A known assay used to measure glutathione reductase is outlined below. The assay is based on the oxidation of NADPH to NADP⁺ catalysed by a limiting concentration of glutathione reductase. One GR activity unit is defined as the reduction of one micromole of GSSG per minute at pH 7.6 and 25° C. As shown in the above reaction, one molecule of NADPH is consumed for each molecule of GSSG reduced. Therefore, the reduction of GSSG is determined indirectly by the measurement of the consumption of NADPH, as demonstrated by a decrease in absorbance at 340 nm (A340) as a function of time. A standard curve is created by adding increasing amounts of gluthathione reductase.

| NADPH | GSSG | KPO4 | DILUENT |
|---|---|---|---|
| 3.8 μmol NADPH | 2.4 mM Oxidised | 125 mM potassium | 50 mM potassium |
| (reduced) at 25° C. | glutathione, pH 7.5. | phosphate, pH 7.5, | phosphate, pH 7.5, at |
| | | at 25° C. | at 25° C. |
| 20 μmol TRIS | 125 mM potassium | 2.5 mM EDTA | 1 mM EDTA |
| | phosphate | | |
| 10 mg mannitol | 2.5 mM EDTA | | 1 mg/mL BSA |

The assay procedure is as follows. Pipette into a cuvette 200 μL of sample, 400 μL GSSG (or KPO₄ for a sample Blank), 400 μL NADPH. The solution is mixed and incubated. Results are obtained by recording in a spectrophotometer the A340 for a minimum of five minutes.

Calculation of the rate of decrease in the A340 per minute is obtained by (a) averaging the dA340/dt where dt=time interval in minutes; (b) performing linear regression of the A340 as a function of time; or (c) automatic calculations using the spectrophotometer, if available. The net rate for the sample is calculated by subtracting the rate obtained for the Blank

The concentration of GR is expressed in units of activity. One GR unit will reduce one μmol of GSSG per minute at 25° C. and pH 7.6; therefore, the decrease in GSSG is equal to the consumption of NADPH, measured as the decrease in the absorbance at 340 nm. The molar extinction coefficient (e) for NADPH is 6220 M⁻¹ cm⁻¹.

Assay Range.

Glutathione reductase samples should be diluted to provide a minimum net rate of 0.0050 A340 per minute (10× the typical blank rate) and a maximum of 0.0625 A340/min. This corresponds to approximately 0.8 to 10.0 mU/mL final concentration in the assay. Lower concentrations may not provide sufficient dA340 in a five-minute interval and excess GR may cause the rate to be non-linear.

What is claimed is:

1. A method to treat, prevent or slow the progression of a degenerative disorder, a neurodegenerative disorder, a degenerative-related disorder, a neurodegenerative-related disorder, malaria, a leishmania parasite infection or a trypanosome infection, or to ameliorate a symptom thereof, or to treat aluminum intoxication, reperfusion injury, or to reduce the level of iron or to reduce free transition metal ion levels in the body or in certain body compartments, in a subject in need thereof, the method comprising administering to the subject or delivering to the subject's tissues a therapeutically effective amount of a compound having the formula 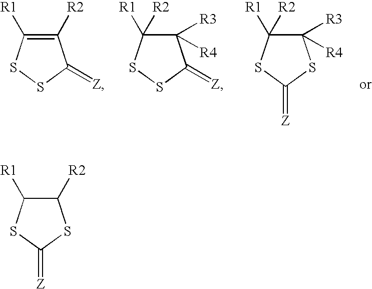
and oxides, derivatives and metabolites thereof, wherein
Z is S, O, NR, R₂ or CR₂;
R is —H, —OH, C₁-C₅ alkyl, C₁-C₅ alkoxy or C₁-C₅ alkoxycarbonyl;
R₂, together with the atoms to which it is bonded, comprises a spiro or fused ring to yield a bicyclic or tricyclic compound, which is saturated or unsaturated, heterocyclic or carbocyclic and wherein the rings are all optionally substituted 5-, 6-, 7- or 8-membered rings, with substituents optionally selected from C₁-C₄ alkyl, C₁-C₄ alkoxy, —SO₃H, —OH and halogen;
R1, R2, R3 and R4 independently are —H, -alkyl, -aryl, -alkylaryl, a heterocycle, a halogen, -alkoxycarbonyl (C₁-C₅) or -carboxyl,
wherein either alkyl is a C₁-C₁₀ linear or branched chain, saturated or unsaturated moiety, which is optionally substituted by 1, 2 or more independently selected ether (—O—), halogen, alkyl (C₁-C₅), —OH, alkoxy (C₁-C₅), alkoxycarbonyl, (C₁-C₅), carboxyl, amido, alkyl amido (C₁-C₅), amino, mono- or dialkylamino (C₁-C₅), alkyl carbamoyl (C₁-C₅), thiol, alkylthio (C₁-C₅), or benzenoid aryl, and
wherein the -aryl and -alkylaryl substituent for R1, R2, R3 and R4 comprises a benzenoid group (C₆-C₁₄), wherein the benzenoid group is optionally substituted with 1, 2 or more independently selected —SO₃H, halogen, alkyl (C₁-C₅), —OH, alkoxy (C₁-C₅), alkoxycarbonyl, (C₁-C₅), carboxyl, amido, alkyl amido (C₁-C₅), amino, mono- or dialkylamino (C₁-C₅), alkyl carbamoyl (C₁-C₅), thiol, alkylthio (C₁-C₅), and
wherein the heterocycle is defined as any 4, 5 or 6 membered, optionally substituted heterocyclic ring, saturated or unsaturated, containing 1-3 ring atoms selected from N, O and S, the remaining ring atoms being carbon; and wherein said substituents on said aryl or said heterocyclic are selected from the group consisting of halogen, alkyl (C₁-C₅), hydroxyl, alkoxy (C₁-C₅), alkoxycarbonyl (C₁-C₅), carboxyl, amido, alkyl amido (C₁-C₅), amino, mono and dialkyl amino (C₁-C₅), alkyl carbamoyl (C₁-C₅), thiol, alkylthio (C₁-C₅), benzenoid, aryl, cyano, nitro, haloalkyl (C₁-C₅), alklsulfonyl (C₁-C₅), or sulfonate, or
one of R1 and R2 and one of R3 and R4 together with the carbon atoms to which they are attached comprise a fused bicyclic or tricyclic compound, which is saturated or unsaturated, heterocyclic or carbocyclic and wherein the rings are all optionally substituted 5-, 6-, 7- or 8-membered rings, with substituents optionally selected from alkyl, alkoxy, —SO₃H, —OH and halogen, or
R1 and R2 together or R3 and R4 together independently are oxime (═NOH).

2. The method of claim 1 wherein the compound is selected from the group consisting of oltipraz, 5-(4-methoxyphenyl)-3H-1,2-dithiole-3-thione, ADT, ADO, 1,2-dithiole-3-thione, 1,2-dithiolane, 1,3-dithiole-2-thione, and malotilate.

3. The method of claim 1 wherein the compound chelates with, or forms a complex with, one or more divalent or trivalent metal ions, whereby the divalent or trivalent ions in the subject's cells or tissues are redistributed or sequestered such that the ions are limited in their capacity to participate in unwanted reactions such as the Fenton reaction.

4. The method of claim 3 wherein the divalent or trivalent metal ions are selected from Fe, Cu, Ni, Ca, Mg, Mn, Cd, Pb, Al, Hg, Co and Zn ions.

5. The method of claim 4 wherein the divalent or trivalent metal ion is Fe or Cu.

6. The method of claim 1 wherein the degenerative disorder, neurodegenerative disorder, degenerative-related disorder or neurodegenerative-related disorder is selected from the group consisting of Parkinson's disease, Hungtington's disease, Amylotrophic Lateral Sclerosis, Cerebral amyloid angiopathy, Multiple Sclerosis, cognitive disorders, Progeria, Alzheimer's disease, epileptic dementia, presenile dementia, post traumatic dementia, senile dementia, vascular dementia, HIV-1-associated dementia, post-stroke dementia, Down's syndrome, motor neuron disease, amyloidosis, amyloid associated with type 11 diabetes, Creutzfelt-Jakob disease, necrotic cell death, Gerstmann-Straussler syndrome, kuru and animal scrapie, amyloid associated with long-term hemodialysis, senile cardiac amyloid and Familial Amyloidotic Polyneuropathy, cerebropathy, neurospanchnic disorders, memory loss and related degenerative disorders.

7. The method of claim 1 wherein the compound is oltipraz and the neurodegenerative disorder is Alzheimer's disease.

8. The method of claim 1 wherein said compound is a D-amino acid oxidase inhibitor and cellular degeneration is slowed or arrested.

9. The method of claim 1 wherein said compound enhances one or more phase II detoxification enzymes.

10. The method of claim 9 wherein said phase II detoxification is selected from the group consisting of glutathione S transferase, γ-glutamylcysteine synthetase, glutathione reductase, glutathione peroxidase, epoxide hydrase, AFB₁ aldehyde reductase, glucuronyl reductase, glucose-6-phosphate dehydrogenase, UDP-glucuronyl transferase, and AND(P)H:quinone oxidoreductase.

11. A method to treat, prevent or slow the progression of a degenerative disorder, a neurodegenerative disorder, a degenerative-related disorder, a neurodegenerative-related disorder, malaria, a leishmania parasite infection or a trypanosome infection, or to ameliorate a symptom thereof, or to treat aluminum intoxication, reperfusion injury, or to reduce the level of iron or to reduce free transition metal ion levels in the body or in certain body compartments, in a subject in need thereof, the method comprising administering to the subject or delivering to the subject's tissues a therapeutically effective amount of a compound having the formula selected from the group consisting of (1), (2), (3) and (4); 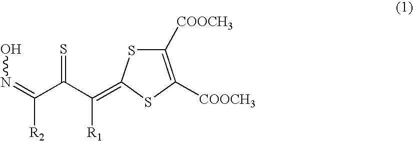
wherein R₁ and R₂ are each independently selected from the group consisting of hydrogen, halogen, nitro, nitroso, thiocyano, C₁-C₆ alkyl, C₂-C₆ alkenyl, aryl, aryl(C₁-C₆ alkyl), aryl(C₂-C₆ alkenyl), carboxyl, (C₁-C₆ alkyl)carbonyl, arylcarbonyl, (C₁-C₆ alkoxy)carbonyl, (C₁-C₆ alkoxy)carbonyl(C_{1,}-C₆ alkyl), C₁-C₆ alkoxy, trifluoromethyl, amino, di(C₁-C₆ alkyl)amino(C₁-C₆ alkyl), —NHCOCₙH₂ₙ₊₁ with n from 0 to 6, —NH—CSCₙH₂ₙ₊₁ with n from 0 to 6, terpenyl, cyano, C₂-C₆ alkynyl, C₂-C₆ alkynyl substituted with a C₁-C₆ alkyl or aryl, hydroxy(C₁-C₆ alkyl), a (C₁-C₆ acyl)oxy(C₁-C₆ alkyl), (C₁-C₆ alkyl)thio and arylthio group, or alternatively R₁ and R₂ together form a mono- or poly-cyclic C₂-C₂₀ alkylene-group optionally comprising one or more hetero atoms and wherein
the aryl group or aryl fraction of said arylalkyl group denotes an aromatic carbon-based group or an aromatic heterocyclic group optionally substituted with one, two or more substituents independently chosen from halogen, C₁-C₄ alky, C₁-C₄ alkoxy group, a trifluoromethyl group, a nitro group and a hydroxyl group; 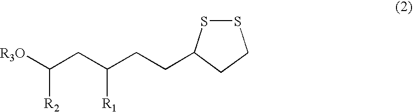
wherein R₁ and R₂ are each independently oxygen (—O) or —OR, where R is H or C₁-C₄ alkyl; and wherein R₃ is H, Na, K or (C₁-C₄) alkyl; 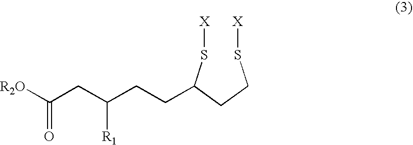
wherein X is H or both Xs represent a direct bond between the two sulfur atoms;
R₁ is ═O or —OH; and R₂ is H, Na, K or C₁-C₄ alkyl; and 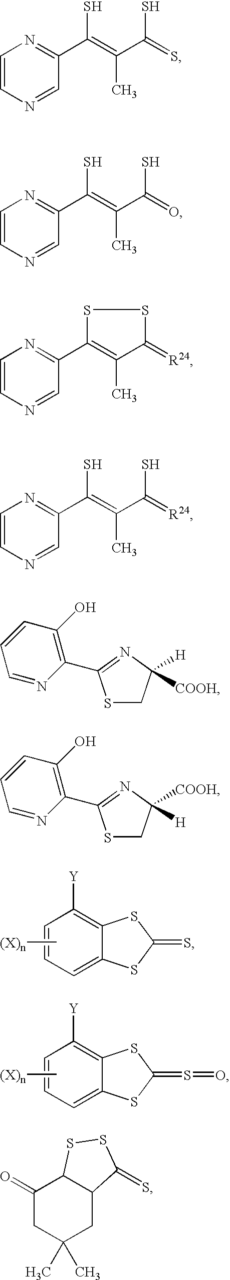 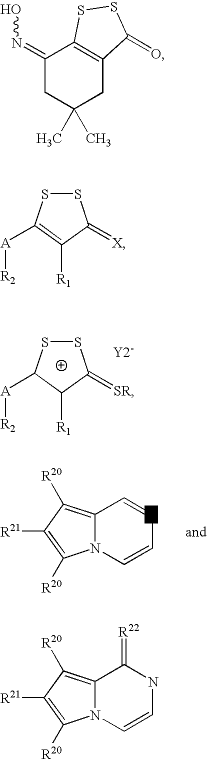
wherein
R is C₁-C₆ alkyl;
R₁ and R₂ independently are hydrogen, a halogen, nitro, nitroso, a thiocyano group, a C₁-C₆ alkyl group, a C₂-C₆ alkenyl group, an aryl group, aryl (C₁-C₆ alkyl) group, an aryl (C₂-C₆ alkenyl) group, a carboxyl group, a (C₁-C₆ alkyl) carbonyl group, an aryl carbonyl group, a (C₁-C₆ alkoxy)carbonyl group, a (C₁-C₆ alkoxy)carbonyl (C_{1,}-C₆ alkyl) group, a C₁-C₆ alkoxy group, a trifluoromethyl group, an amino group, a di(C₁-C₆ alkyl) amino(C₁-C₆ alkyl) group, an acylamino group of formula —NHCOCₙH₂ₙ₊₁ with n from 0 to 6, a group —NH—CSCₙH₂ₙ₊₁ with n from 0 to 6, a terpenyl group, a cyano group, a C₂-C₆ alkynyl group, a C₂-C₆ alkynyl group substituted with a C₁-C₆, alkyl or an aryl group, a hydroxy(C₁-C₆ alkyl) group, a (C₁-C₆ acyl) oxy (C₁-C₆ alkyl) group, a (C₁-C₆ alkyl) thio group and an arylthio group, or R₁ and R₂ together comprise a mono- or poly-cyclic C₂-C₂₀ alkylene group optionally comprising one or more hetero atoms, but they are not 2,2 dimethyltrimethylene, or C₃-C₁₂ cycloalkylene;
R₃ is hydroxyl, amino, chloro, C_{1,}-C₄, alkoxy, aryl-C_{1,}-C₆ alkyl, a (C₁-C₆ alkyl)carbonyl group or R₃ is an aryl (C₁-C₆ alkyl) carbonyl group) or A is —CHOH, >C═O or >C═N—R₄, where R₄ is C₁-C₆ alkyl or aryl group;
R₅, is C₁-C₆ alkyl or aryl;
R²⁰ independently is —SH, —SCH₃, —S(O)CH₃, —OH, —OCH₃, —S—C1-C6 alkyl opotionally substituted with 1, 2 or more independently selected —O—, —S—, —OH, halogen, —CN, ═O or —C(O)—NH— moieties, or R²⁰ independently is —S—C1-C6 alkyl opotionally substituted with 1, 2 or more independently selected —O—, —S—, —OH, halogen, —CN, ═O or —C(O)—NH— moieties;
R²¹ is C1-C6 alkyl; and
R²² is ═O or ═S;
R²⁴ is ═S, ═O, ═N—OH, ═N—R₅, ═N—NH—CO—NH₂, ═N—NH—CS—NH₂, or ═CZZ′;
A is oxime or >C═N—OR₃;
n is an integer from 1 to 3;
Y is selected from nitro and trifluoromethyl; X is selected from alkyl and alkenyl of up to 6 carbon atoms, nitro, trichloromethyl, trifluoromethyl, trifluoromethoxy, trifluoromethylthio, trifluoromethylsulfoxyl, trifluoromethylsulfonyl, methoxymethyl, cyano, carboxy, halogen, hydroxy, acetylamino, amino, N-phenylamino, N,N-diallylamino, C₁-C₅ alkoxy, N-morpholino, N-piperidino, N-piperazino, N-pyrrolidino, dimethylaminodithiocarbarnyl, carboalkoxy, alkylthio, mono- and dialkylamino, N-alkyl-carbamyl, N,N-dialkylcarbamyl, alkylsulfoxy, and alkylsulfonyl, said alkyl groups containing 1, 2, 3 or 4 carbon atoms; and at least one of said X groups is selected from N-morpholino, N-piperidino, N-piperazino or N-pyrrolidino;
Y2 is an acceptable anion; and
Z and Z′ independently are —H or an electron-attracting group; and pharmaceutically acceptable salts thereof.

12. The method of claim 1 wherein the compound chelates with, or forms a complex with, one or more divalent or trivalent metal ions, whereby the divalent or trivalent ions in the subject's cells or tissues are redistributed or sequestered such that the ions are limited in their capacity to participate in unwanted reactions such as the Fenton reaction.

13. The method of claim 3 wherein the divalent or trivalent metal ions are selected from Fe, Cu, Ni, Ca, Mg, Mn, Cd, Pb, Al, Hg, Co and Zn ions.

14. The method of claim 4 wherein the divalent or trivalent metal ion is Fe or Cu.

15. The method of claim 11 wherein the degenerative disorder, neurodegenerative disorder, degenerative-related disorder or neurodegenerative-related disorder is selected from the group consisting of Parkinson's disease, Hungtington's disease, Amylotrophic Lateral Sclerosis, Cerebral amyloid angiopathy, Multiple Sclerosis, cognitive disorders, Progeria, Alzheimer's disease, epileptic dementia, presenile dementia, post traumatic dementia, senile dementia, vascular dementia, HIV-1-associated dementia, post-stroke dementia, Down's syndrome, motor neuron disease, amyloidosis, amyloid associated with type 11 diabetes, Creutzfelt-Jakob disease, necrotic cell death, Gerstmann-Straussler syndrome, kuru and animal scrapie, amyloid associated with long-term hemodialysis, senile cardiac amyloid and Familial Amyloidotic Polyneuropathy, cerebropathy, neurospanchnic disorders, memory loss and related degenerative disorders.

16. The method of claim 1 or claim 11 wherein the compound micronized or the compound is present in a composition that comprises a pharmaceutically acceptable carrier, the carrier optionally selected from phosphatidylcholine, diphosphatidylcholine, vitamin E, a cyclodextrin, magnolol, a microbial preservative, water or a liquid excipient suitable for ophthalmic pharmaceutical formulations.

17. The method of claim 11 wherein said compound is a D-amino acid oxidase inhibitor and cellular degeneration is slowed or arrested.

18. The method of claim 11 wherein said compound enhances a phase II detoxification enzyme.

19. The method of claim 18 wherein said phase II detoxification enzyme is selected from the group consisting of glutathione S transferase, γ-glutamylcysteine synthetase, glutathione reductase, glutathione peroxidase, epoxide hydrase, AFB₁ aldehyde reductase, glucuronyl reductase; glucose-6-phosphate dehydrogenase, UDP-glucuronyl transferase and AND(P)H:quinone oxidoreductase.

20. The method of claim 1, wherein the compound is 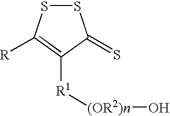
wherein
R is —H or C₁ to C₁₂ alkyl;
R¹ is C₆ to C₁₂ arylene;
R² is C₁ to C₄ alkylene; and
n is 2 to 50; 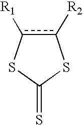
wherein the dotted line is an optional; bouble bond and R₁ and R₂ are independently selected from the group consisting of hydrogen; C₁₋₂₀ alkyl groups and C₂₋₁₂ alkenyl groups; 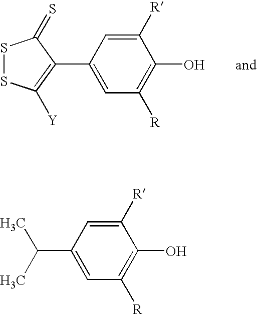
wherein
R and R′ independently are C1-C12 alkyl or C3-C12 cycloalkyl, either of which are optionally substituted with C1-C4 alkyl or an aralkyl radical having from 7 to 14 carbon atoms;
Y is —H or —SH; and
R′ is C1-C20 alkyl, C5-C12 cycloalkyl, C3-C20 alkenyl, C7-C14 aralkyl.

21. The method of claim 20 which comprises administering or delivering to the subject a therapeutically effective amount of a compound selected from the group consisting of:
4-(3,5-diisopropyl-4-hydroxyphenyl)-1,2-dithiole-3-thione;
4-(3,5-di-t-butyl-4-hydroxyphenyl)-1,2-dithiole-3-thione;
4-[3,5-bis(l,l-dimethylpropyl)-4-hydroxyphenyl]-1,2-dithi-ole-3-thione;
4-[3,5bis(l,l-dimethylbutyl)-4-hydroxyphenyl]-1,2-dithiole-3-thione;
4-[3,5-bis(1,1,3,3-tetramethylbutyl)-4-hydroxyphenyl]-1,2-dithole-3-thione;
4-[3,5-bis(l-methylcyclohexyl)-4-hydroxyphenyl]-1,2-dithiole-3-thione;
4-[3,5-bis(l,l-dimethylbenzyl)-4-hydroxyphenyl]-1,2-dithiole-3-thione;
4-(3t-butyl-4-hydroxy-S-isopropylphenyl)-1,2-dithiole-3-thione;
4-(3t-butyl-4-hydroxy-5-methylphenyl)-1,2-dithiole-3-thione;
4-[3(1,1-dimethylpropyl)-4-hydroxy.-5-isopropylphenyl]-1,2-dithiole-3-thione;
4-[3(1,1-dimethylbenzyl)-4-hydroxy-5-isopropylphenyl]-1,2-dithole-3-thione;
5-benzylthio-4-(3,5-di-t-butyl-4-hydroxyphenyl)-1,2-dithole-3-thione;
5-benzylthio-4-[3,5-bis(l,l-dimethylpropyl)-4-hydroxy-phenyl]-1,2-dithiole-3-thione;
5-hexylthio-4-(3,5-di-t-butyl-4-hydroxyphenyl)-1,2-dithole-3-thione;
5-hexylthio-4-[3,5-bis(l,l-dimethylbutyl)-4-hydroxy-phenyl]-1,2-dithole-3-thione;
5-octadecylthio-4-(3,5-di-t-butyl-4-hydroxyphenyl)-1,2-dithiole-3-thione;
5-octadecylthio-4-[3,5-bis(l,l-dimethylbenzyl)-4-hydroxyphenyl]-1,2-dithiole-3-thione;
5-allylthio-4-(3,5-di-t-butyl-4-hydroxyphenyl)-1,2-dithiole-3-thione;
5-cyclohexylthio-4-(3,5-di-t-butyl-4-hydroxyphenyl)-1,2-dithiole-3-thione; and 4-(3,5-di-sec-butyl-4-hydroxyphenyl)-1,2-dithiole -3-thione.

22. The method of claim 20 wherein the compound chelates with, or forms a complex with, one or more divalent or trivalent metal ions, whereby the divalent or trivalent ions in the subject's cells or tissues are redistributed or sequestered such that the ions are limited in their capacity to participate in unwanted reactions such as the Fenton reaction.

23. The method of claim 22 wherein the divalent or trivalent metal ions are selected from Fe, Cu, Ni, Ca, Mg, Mn, Cd, Pb, Al, Hg, Co and Zn ions.

24. The method of claim 20 wherein the compound is oltipraz and the neurodegenerative disorder is Alzheimer's disease.

25. The method of claim 20 wherein the degenerative disorder, neurodegenerative disorder, degenerative-related disorder or neurodegenerative-related disorder is selected from the group consisting of Parkinson's disease, Hungtington's disease, Amylotrophic Lateral Sclerosis, Cerebral amyloid angiopathy, Multiple Sclerosis, cognitive disorders, Progeria, Alzheimer's disease, epileptic dementia, presenile dementia, post traumatic dementia, senile dementia, vascular dementia, HIV-1-associated dementia, post-stroke dementia, Down's syndrome, motor neuron disease, amyloidosis, amyloid associated with type II diabetes, Creutzfelt-Jakob disease, necrotic cell death, Gerstmann-Straussler syndrome, kuru and animal scrapie, amyloid associated with long-term hemodialysis, senile cardiac amyloid and Familial Amyloidotic Polyneuropathy, cerebropathy, neurospanchnic disorders, memory loss and related degenerative disorders.

26. The method of claim 20 wherein said compound is formulated into a composition that further comprises a pharmaceutically acceptable carrier.

27. The method of claim 20 wherein said compound is a D-amino acid oxidase inhibitor and cellular degeneration is slowed or arrested.

28. The method of claim 20 wherein said compound enhances a phase II detoxification enzyme.

29. The method of claim 28 wherein said phase II detoxification is selected from the group consisting of glutathione S transferase, γ-glutamylcysteine synthetase, glutathione reductase, glutathione peroxidase, epoxide hydrase, AFB₁ aldehyde reductase, glucuronyl reductase; glucose-6-phosphate dehydrogenase, UDP-glucuronyl transferase and AND(P)H:quinone oxidoreductase.

30. A method to treat, prevent or slow the progression of a degenerative disorder, a neurodegenerative disorder, a degenerative-related disorder, a neurodegenerative-related disorder, malaria, a leishmania infection or a trypanosome infection, or to ameliorate a symptom thereof, or to treat aluminum intoxication, reperfusion injury, or to reduce the level of iron or to reduce free transition metal ion levels in the body or in certain body compartments, in a subject in need thereof, the method comprising administering to the subject or delivering to the subject's tissues a therapeutically effective amount of a compound having the formula 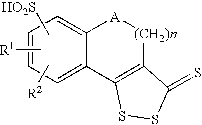
wherein A is a methylene group or an oxygen atom;
R¹ and R² are each independently —H, —OH, a halogen, lower alkyl or lower alkoxy; and
n is 0, 1, 2 or 3 when A is a methylene group, and n is 1, 2 or 3 when A is an oxygen atom; or a salt thereof;
or wherein the compound has the formula 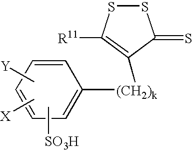
wherein
k is 0, 1, 2, 3, 4 or 5;
X and Y are independently —H, lower alkyl or lower alkoxy;
R¹¹ is an alkyl group or 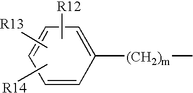
where m is an integer of 0-4; and R12, R13 and R14 are each independently a hydrogen atom, C₁-C₄ alkyl or C₁-C₄ alkoxy, or a salt thereof, but excluding the compound where k and m are both 0, the sulfo group is bonded to the 3-position, X is 4-methoxy and R12, R13, R14 and Y are all hydrogen.

31. The method of claim 30 wherein the compound is selected from the group consisting of:

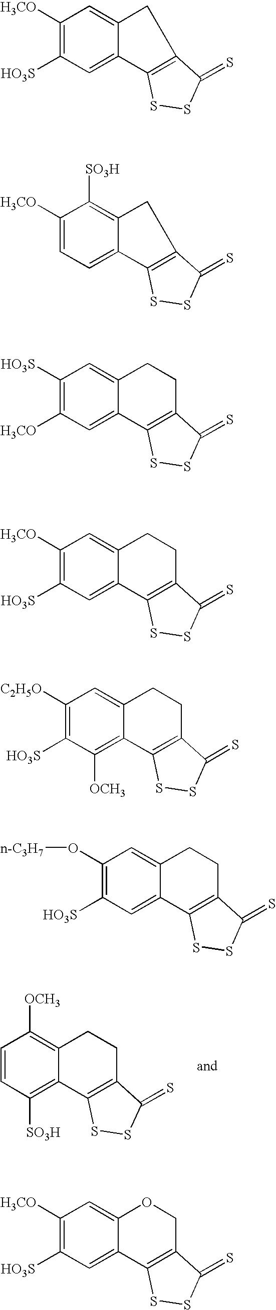

32. The method of claim 31 wherein the compound is
5-hexyl-4-(4-methoxy-3-sulfobenzyl)-3H-1,2-dithole-3-thione,
4-(4-methoxy-3-sulfophenyl)-5-(p-toluyl)-3H-1,2-dithiole-3-thione, or a salt thereof.

33. The method of claim 30 wherein the compound chelates with, or forms a complex with, one or more divalent or trivalent metal ions, whereby the divalent or trivalent ions in the subject's cells or tissues are redistributed or sequestered such that the ions are limited in their capacity to participate in unwanted reactions such as the Fenton reaction.

34. The method of claim 30 wherein the divalent or trivalent metal ions are selected from Fe, Cu, Ni, Ca, Mg, Mn, Cd, Pb, Al, Hg, Co and Zn ions.

35. The method of claim 30 wherein the compound is an oxime or a derivative of said compound.

36. The method of claim 30 wherein said degenerative disorder, neurodegenerative disorder, degenerative-related disorder or neurodegenerative-related disorder is selected from the group consisting of Alzheimer's disease, Parkinson's disease, Huntington's disease, Amylotrophic Lateral Sclerosis, Cerebral amyloid angiopathy, Multiple Sclerosis, cognitive disorders, Progeria, epileptic dementia, presenile dementia, post traumatic dementia, senile dementia, vascular dementia, HIV-1-associated dementia, post-stroke dementia, Down's syndrome, motor neuron disease, amyloidosis, amyloid associated with type II diabetes, Creutzfelt-Jakob disease, necrotic cell death, Gerstmann-Straussler syndrome, kuru and animal scrapie, amyloid associated with long-term hemodialysis, senile cardiac amyloid and Familial Amyloidotic Polyneuropathy, cerebropathy, neurospanchnic disorders, memory loss, aluminum intoxication, reperfusion injury, reducing the level of iron in the cells of living subjects, reducing free transition metal ion levels in mammals, patients having toxic amounts of metal in the body or in certain body compartments, and related degenerative disorders.

37. The method of claim 30 wherein said compound is formulated into a composition that further comprises a pharmaceutically acceptable carrier.

38. The method of claim 30 wherein said compound is a D-amino acid oxidase inhibitor.

39. The method of claim 30 wherein said compound enhances one or more phase II detoxification enzymes.

40. The method of claim 39 wherein said phase II detoxification is selected from the group consisting of glutathione S transferase, γ-glutamylcysteine synthetase, glutathione reductase, glutathione peroxidase, epoxide hydrase, AFB₁ aldehyde reductase, glucuronyl reductase; glucose-6-phosphate dehydrogenase, UDP-glucuronyl transferase and AND(P)H:quinone oxidoreductase.

41. The method of claim 1 wherein the compound comprises at least one adjunct residue that is covalently bonded to the compound, and the adjunct residue is optionally comprises one to eighty amino acids, which optionally comprise positively charged amino acids.

42. The method of embodiment 41 wherein the positively charged amino acids independently are histidine, arginine or lysine.

43. The method of claim 11 wherein the compound comprises at least one adjunct residue that is covalently bonded to the compound, and the adjunct residue is optionally comprises one to eighty amino acids, which optionally comprise positively charged amino acids.

44. The method of embodiment 43 wherein the positively charged amino acids independently are histidine, arginine or lysine.

45. A method of making oltipraz comprising
esterifying pyrazine-2-carboxylic acid with methanol in the presence of an acid to form methyl-pyrazine-2-carboxylate;
condensing said methyl-pyrazine-2-carboxylate with methyl propionate in the presence of a base to form methyl-2-methyl-3-(pyrazin-2-yl)-3-oxopropionate; and
treating said methyl-2-methyl-3-(pyrazin-2-yl)-3-oxopropionate with phosphorus pentasulfide to form oltipraz.

46. The method of claim 45 wherein said acid is sulfuric acid and said base is sodium hydride or potassium hydride.

47. The method of claim 45 wherein said steps (b) and (c) are conducted in the presence of an aromatic hydrocarbon.

48. The method of claim 47 wherein said aromatic hydrocarbon is toluene.

49. A method to determine if a mammal has a degenerative disorder, a neurodegenerative disorder, a degenerative-related disorder, a neurodegenerative-related disorder, or the propensity to develop such a disorder, comprising:
(a) obtaining a circulatory fluid sample from the mammal;
(b) splitting the circulatory fluid sample into two, three or more suitable aliquots;
(c) determining the hydrogen peroxide level in a first aliquot;
(d) contacting a second aliquot with a sufficient amount of a one, two or more D-amino acids;
(e) incubating the second aliquot for sufficient timeland under conditions suitable to allow detectable metabolism of the one, two or more D-amino acids to determine the level of hydrogen peroxide in the second aliquot;
(f) determining the hydrogen peroxide level of second first aliquot; and
(g) comparing the hydrogen peroxide level obtained from step (c) and step (f) and the, whereby a high hydrogen peroxide level indicates the presence of a neurodegenerative or related disorder or the propensity to develop such a disorder.

50. The method of claim 49, wherein the mammal is a human.

51. The method of claim 49, wherein the circulatory fluid is blood, plasma, serum or spinal fluid.

52. The method of claim 49 wherein the neurodegenerative disorder is Alzheimer's disease.

53. A method to determine if a mammal has a degenerative disorder, a neurodegenerative disorder, a degenerative-related disorder, a neurodegenerative-related disorder, or a propensity to develop such a disorder, comprising:
(a) obtaining a circulatory fluid sample from the mammal; and
(b) determining a hydrogen peroxide level in circulatory fluid sample;
(c) determining the D-amino acid oxidase level in the circulatory fluid sample using the hydrogen peroxide level in step (b);
(d) comparing the D-amino acid oxidase level in the circulatory fluid from step (c) with a D-amino acid oxidase level in the circulatory fluid of a healthy control mammal(s), whereby an increased D-amino acid oxidase level in the circulatory fluid indicates the presence of or propensity to develop the degenerative or related disorder.

54. The method of claim 53, wherein the mammal is a human.

55. The assay of claim 53, wherein the circulatory fluid is blood, plasma, serum or spinal fluid.

56. The method of claim 53 wherein the neurodegenerative disorder is Alzheimer's disease.

57. A method to determine if a mammal has a degenerative disorder, a neurodegenerative disorder, a degenerative-related disorder, a neurodegenerative-related disorder, or a propensity to develop such a disorder, comprising measuring the mammal's D-amino acid oxidase level and comparing the result to that obtained from a control mammal(s) with no degenerative or related disorder or a propensity to develop such a disorder.

58. The method of claim 53 wherein mammal's D-amino acid oxidase level is measured by determining a relative activity of the mammal's anti-oxidative enzymes compared to a control mammal(s) with no degenerative or related disorder or a propensity to develop such a disorder.

59. The method of claim 54 wherein the relative activity of the mammal's anti-oxidative enzymes is determined by quantitative PCR analysis of RNA that encodes the mammal's anti-oxidative enzymes compared to the control mammal(s), wherein a decreased level of RNA that encodes the mammal's anti-oxidative enzymes compared to the control mammal's level of the same RNA indicates the presence of the degenerative or related disorder or a propensity to develop the disorder.

60. The method of claim 59 wherein the mammal's RNA level is at least about 1.4-fold to about 3-fold higher than the control mammal's level of the same RNA.

61. The method of claim 59 wherein the anti-oxidative enzyme is glutathione S transferase, γ-glutamylcysteine synthetase, glutathione reductase, glutathione peroxidase, epoxide hydrase, AFB₁ aldehyde reductase, glucuronyl reductase; glucose-6-phosphate dehydrogenase, UDP-glucuronyl transferase or AND(P)H:quinone oxidoreductase.

62. Use of one or more of the compounds of claim 1 or claim 11 for the manufacture of a medicament for a degenerative disorder, a neurodegenerative disorder, a degenerative-related disorder, a neurodegenerative-related disorder, or of treatment of malaria, a leishmania infection, or a trypanosome infection.

63. Use in a method of treatment of degenerative or related disorders, or of treatment of malaria or a trypanosome infection, said method comprising administering an effective amount of one or more to of the compounds of claim 1 or claim 11 a subject in need thereof.

64. Use of a D-amino acid oxidase inhibitor to treat or prevent a degenerative disorder, a neurodegenerative disorder, a degenerative-related disorder, a neurodegenerative-reiated disorder, comprising administering to a mammal in need thereof an effective amount of the D-amino acid oxidase inhibitor.

65. A composition comprising a pharmaceutically acceptable carrier and a compound of the formula 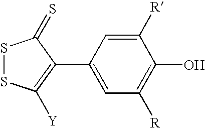
wherein R and R′ independently are the same or different and each is C1-C12 alkyl or C5-C12 cycloalkyl, either of which are optionally substituted with C1-C4 alkyl or C7-C14 aralkyl; and
Y is —H, —SH or —SR² where R² is C1-C20 alkyl radical, C5-C12 cycloalkyl, C3-C20 alkenyl, or C7-C14 aralkyl.

66. The composition of claim 65 wherein
(1) R and R¹ are branched-chain alkyl radicals having from 3 to 8 carbon atoms, 1-methyl cyclohexyl or αα-dimethyl benzyl;
(2) Y is an —S-alkyl group having from 6 to 18 carbon atoms; or
(3) the compound is 4-(3,5-di-isopropyl-4-hydroxyphenyl)-1,2-dithiole-3-thione, 4-((3,5-di-t-butyl-4-hydroxyphenyl)-1,2-dithiole-3-thione, 4-[3,5-bis(1,1-dimethyl propyl)-4-hydroxyphenyl]-1,2-dithiole-3-thione, 4-[3,5-bis(1,1-dimethylbutyl)-4-hydroxyphenyl]-1,2-dithiole-3-thione, 4-[3,5-bis(1,1,3,3-tetramethylbutyl)-4-hydroxyphenyl]4-[3,5-bis(1-methylcyclohexyl)-4-hydroxyphenyl]-1,2-dithiole-3-thione, 4-[3,5-bis(1,1-dimethylbenzyl)-4-hydroxyphenyl]-1,2-dithiole-3-thione, 4-(3-t-butyl-4-hydroxy-5-isopropylphenyl)-1,2-dithiole-3-thione, 4-(3-t-butyl-4-hydroxy-5-methylphenyl)-1,2-dithiole-3-thione, 4-[3-(1,1-dimethylpropyl)-4-hydroxy-5-isopropylphenyl]-1,2-dithole-3-thione, 4-[3-(1,1-dimethylbenzyl)-4-hydroxy-5-isopropylphenyl]-1,2-dithiole-3-thione, 5-benzylthio-4-(3,5-di-t-butyl-4-hydroxyphenyl)-1,2-dithiole-3-thione, 5-benzylthio-4-[3,5-bis(1,1-dimethylpropyl)-4-hydroxy-phenyl]-1,2-dithiole-3-thione, 5-hexylthio-4-(3,5-di-t-butyl-4-hydroxyphenyl)-1,2-dithiole-3-thione, 5-hexylthio-4-[3,5-bis(1,1-dimethylbutyl)-4-hydroxy-phenyl]-1,2-dithiole-3-thione, 5-octadecylthio-4-(3,5-di-t-butyl-4-hydroxyphenyl)-1,2-dithiole-3-thione, 5-octadecylthio-4-[3,5-bis(1,1-dimethylbenzyl)-4-hydroxyphenyl]-1,2-dithiole-3-thione, 5-allylthio-4-(3,5-di-t-butyl-4-hydroxyphenyl)-1,2-dithiole-3-thione, 5-cyclohexylthio-4-(3,5-di-t-butyl-4-hydroxyphenyl)-1,2-dithiole-3-thione or 4-(3,5-di-sec-butyl-4-hydroxyphenyl)-1,2-dithiole-3-thione.

67. A method to determine if a mammal has a degenerative disorder, a neurodegenerative disorder, a degenerative-related disorder or a neurodegenerative-related disorder, the method comprising
(1) taking a sample of circulatory fluid sample from a subject mammal and from a control mammal;
(2) determining the glutathione reductase levels in each circulatory fluid sample; and
(3) comparing the glutathione reductase levels, whereby a lower glutathione reductase level in the subject mammal compared to the control mammal indicates the presence or probable presence of the neurodegenerative disorder or the neurodegenerative-related disorder.

68. The method of claim 67 wherein the mammal is a human and the neurodegenerative disorder is Alzheimer's disease or Down's syndrome.

69. A method to determine if a mammal has a degenerative disorder, a neurodegenerative disorder, a degenerative-related disorder or a neurodegenerative-related disorder, the method comprising
(1) obtaining a suitable sample from a subject mammal;
(2) quantitatively determining the protein level or the enzyme activity of one or more of the mammal's anti-oxidative enzymes; and
(3) comparing the anti-oxidative enzyme protein or enzyme activity level from step (2) with a suitable normal control mammal, whereby a lower anti-oxidative enzyme protein or enzyme activity level in the subject mammal compared to the control mammal indicates the presence or probable presence of the degenerative disorder, neurodegenerative disorder, degenerative-related disorder or neurodegenerative-related disorder or a propensity to develop such a disorder.

70. The method of claim 69 wherein the anti-oxidative enzyme protein level or the enzyme activity level is one selected from glutathione S transferase, γ-glutamylcysteine synthetase, glutathione reductase, glutathione peroxidase, epoxide hydrase, AFB₁ aldehyde reductase, glucuronyl reductase; glucose-6-phosphate dehydrogenase, UDP-glucuronyl transferase and AND(P)H:quinone oxidoreductase.

71. The method of claim 70 wherein the anti-oxidative enzyme protein level or the enzyme activity level is the glutathione S transferase level.
